(19) United States
(12) Patent Application Publication
(10) Pub. No.: US 2021/0104679 A1
(43) Pub. Date: Apr. 8, 2021

(54) ORGANIC COMPOUND AND ORGANIC ELECTROLUMINESCENCE DEVICE USING THE SAME

(71) Applicants: FENG-WEN YEN, TAIPEI (TW); SHU-HUA YEH, HSINCHU (TW)

(72) Inventors: FENG-WEN YEN, TAIPEI (TW); SHU-HUA YEH, HSINCHU (TW)

(73) Assignee: LUMINESCENCE TECHNOLOGY CORPORATION, HSINCHU (TW)

(21) Appl. No.: 16/591,648

(22) Filed: Oct. 3, 2019

(51) Int. Cl. H01L 51/00; C09K 11/02; C09K 11/06; C07D 495/04; C07D 519/00; C07D 491/048; C07D 487/04

(52) U.S. Cl. CPC H01L 51/0071; H01L 51/5012; C09K 11/06; C07D 495/04; H01L 51/0072; C07D 519/00; H01L 51/0073; C07D 491/048; C07D 487/04; H01L 51/0052; H01L 51/0067; H01L 51/0054; H01L 51/0058; H01L 51/0056; C09K 2211/185; C09K 2211/1029; C09K 2211/1007; C09K 11/02

(57) ABSTRACT

An organic compound is described. An organic electroluminescence device comprises the organic compound as a host or a hole blocking layer. The organic compound of the following formula may lower a driving voltage or increases a current efficiency or a half-life of the organic electroluminescence device.

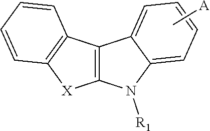

The same definition as described in the present invention.

### FIELD

The present invention relates to an organic compound and, more particularly, to an organic electroluminescence device using the organic compound.

### BACKGROUND

Organic electroluminescence (organic EL) devices, i.e., organic light-emitting diodes (OLEDs) that make use of organic compounds, are becoming increasingly desirable than before. The devices make use of thin organic films that emit light when voltage is applied across the device. They are becoming an interesting technology for use in applications such as flat panel displays, illumination, or backlighting.

One of the organic compounds, denoted H1 hereinafter, has the following structure:

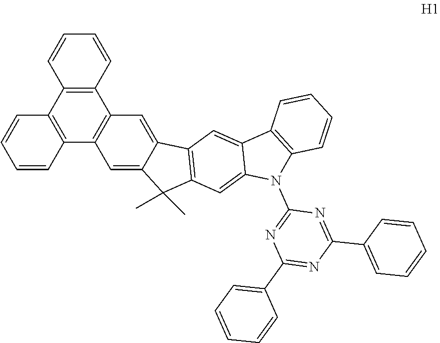 [SMILES: CC1(C)c2cc3c4ccccc4c4ccccc4c3cc2-c2cc3c4ccccc4n(-c4nc(-c5ccccc5)nc(-c5ccccc5)n4)c3cc21]

As used herein, “top” means furthest away from the substrate, while “bottom” means closest to the substrate. Where a second layer is described as formed onto or on a first layer, the second layer is formed further away from substrate. There may be other layers between the second layer and the first layer, unless it is specified that the second layer is “in contact with” the first layer. For example, a cathode may be described as formed onto an anode, even though there are various organic layers in between.

### SUMMARY

An organic compound of formula (1) is disclosed:

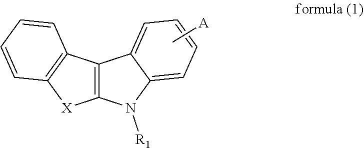

wherein A represents mono to the maximum allowable substitution; wherein each A comprises at least one chemical group selected from the group consisting of

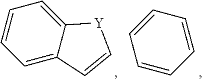

and combinations thereof;
wherein each Y is divalent bridge selected from the group consisting from O, S, CR₇R₈ and NR₉;
wherein X is a divalent bridge selected from the group consisting of O, S and NR₆; and
wherein R₁, R₆, R₇, R₈, and R₉ are independently hydrogen or a substituent selected from the group consisting of alkyl, aralkyl, aralkyl, heteroaryl, deuterium, halide, cycloalkyl, heteroalkyl, arylalkyl, alkoxy, aryloxy, amino, silyl, alkenyl, cycloalkenyl, heteroalkenyl, alkynyl, acyl, carbonyl, carboxylic acids, ester, nitrile, isonitrile, sulfanyl, sulfinyl, sulfonyl, phosphino, and combinations thereof.

An organic EL device is also disclosed. The organic EL device comprises an anode, a cathode and one or more organic layers disposed between the anode and the cathode. At least one of the organic layers comprises the organic compound of formula (1).

### BRIEF DESCRIPTION OF THE DRAWINGS

FIG. 1 is a cross-sectional view of a first organic EL device.

FIG. 2 is a cross-sectional view of an organic EL device without the host 340C of FIG. 1.

FIG. 3 is a cross-sectional view of a second organic EL device. FIG. 1, FIG. 2 and FIG. 3 are not necessarily drawn to scale.

### DETAILED DESCRIPTION

Plural embodiments of the present disclosure are disclosed through drawings. For the purpose of clear illustration, many practical details will be illustrated along with the description below. It should be understood that, however, these practical details should not limit the present disclosure. In other words, in embodiments of the present disclosure, these practical details are not necessary. In addition, for the purpose of simplifying the drawings, some conventional structures and components are simply and schematically depicted in the figures.

It is to be understood that although particular phrases used herein, such as “first”, “second”, “third”, and so on, are used to describe different components, members, regions, layers, and/or sections, these components, members, regions, layers, and/or sections should not be limited by these terms. These phrases are used to distinguish one component, member, region, layer, or section from another component, member, region, layer, or section. In this way, a first component, member, region, layer, and/or section to be described below may be referred to as a second component, member, region, layer, and/or section, without departing from the spirit and scope of the present disclosure.

Spatially relative phrases, such as “onto”, “on”, “under”, “below”, “underlying”, “beneath”, “above”, and so on used herein, are used for facilitating description of a relation between one component or feature and another component or feature depicted in the drawings. Therefore, it can be understood that, in addition to directions depicted in the drawings, the spatially relative terms mean to include all different orientations during usage or operations of the device. For example, it is assumed that a device in a figure is reversed upside down, a component described as being “under”, “below”, or “beneath” another component or feature is oriented “onto” or “on” the other component or feature. Therefore, these exemplary terms “under” and “below” may include orientations above and below. The device may be otherwise oriented (e.g., turned by 90 degrees, or other orientations), and the spatially relative terms used herein should be explained accordingly.

Accordingly, it may be understood that when a component or a layer is referred to as being “onto”, “on”, “connected to”, or “coupled to” another component or another layer, it may be immediately on the other component or layer, or connected to or coupled to the other component or layer, or there may be one or more intermediate components or intermediate layers. Further, it can be understood that when a component or a layer is referred to as being “between” two components or two layers, it may be the only component or layer between the two components or layers, or there may be one or more intermediate components or intermediate layers.

Terminologies used herein are only for the purpose of describing particular embodiments, but not limiting the present disclosure. The singular form of “a” and “the” used herein may also include the plural form, unless otherwise indicated in the context. Accordingly, it can be understood that when there terms “include” or “comprise” are used in the specification, it clearly illustrates the existence of a specified feature, bulk, step, operation, component, and/or member, while not excluding the existence or addition of one or more features, bulks, steps, operations, components, members and/or groups thereof. “And/or” used herein includes any and all combinations of one or more related terms that are listed. When a leading word, such as “at least one of”, is added ahead of a component list, it is to describe the entire component list, but not individual components among the list.

The terms “substituted” and “substitution” refer to a substituent bonded to the relevant position, e.g., a carbon or nitrogen. When R₁ represents no substitution, R₁, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

Generally, an organic EL device comprises at least one organic layer disposed between and electrically connected to an anode and a cathode. When an external voltage is applied across the organic EL device, electrons and holes are injected from the cathode and the anode, respectively. Electrons will be injected from a cathode into a LUMO (lowest unoccupied molecular orbital) and holes will be injected from an anode into a HOMO (highest occupied molecular orbital). Subsequently, the electrons recombine with holes in the light emitting layer to form excitons and then emit light. When luminescent molecules absorb energy to achieve an excited state, the exciton may either be in a singlet state or a triplet state, depending on how the spins of the electrons and holes have been combined.

The term “hydrogen” refers to a —H radical.

The terms “halogen” and “halide” are used interchangeably and refer to fluorine, chlorine, bromine, or iodine.

The term “trifluoromethyl” refers to a —CF₃ radical.

The term “cyano” refers to a —C═N radical.

The term “nitro” refers to a —NO₂ radical.

The term “silyl” refers to a —Si(Rₛ)₃ radical, wherein each Rₛ can be same or different. Rₛ can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combinations thereof. Preferred Rs is selected from the group consisting of alkyl, cycloalkyl, aryl, heteroaryl, and combinations thereof.

As used herein, “a first integer to a second integer” indicates a group comprising at least a first integer, a second integer, and all integers therebetween. For example, “1 to 4 atoms” indicates a group comprising 1, 2, 3 and 4 atoms; and “an integer of 0 to 3” indicates a group comprising 0, 1, 2, and 3.

As used herein, “combinations thereof” indicates that one or more members of the applicable list are combined to form a known or chemically stable arrangement that one of ordinary skill in the art can envision from the applicable list. For example, a monocyclic aromatic group and a polycyclic aromatic group can be combined by being joined through a direct bond, or can be combined to have two carbons common to two adjoining rings (the rings are “fused”); a halogen and alkyl can be combined to form a halogenated alkyl substituent; a halogen, alkyl, and aryl can be combined to form a halogenated arylalkyl; and an alkyl and deuterium can be combined to form a partial or fully deuterated alkyl group. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The term “alkyl” refers to and includes both straight and branched chain alkyl radicals. Preferred alkyl groups are those containing 30 or fewer carbon atoms, preferably 1 to 20 carbon atoms, more preferably 1 to 15 carbon atoms, and most preferably 1 to 12 carbon atoms. Suitable alkyl groups comprise methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “aryl” or “aromatic group” as used herein contemplates a monocyclic aromatic group, a polycyclic aromatic group, and combinations thereof. The polycyclic aromatic group may have two, three, four or more rings in which two carbons are common to two adjoining rings (the rings are “fused”) wherein at least one of the fused rings is an aromatic hydrocarbyl, e.g., the other rings can be cycloalkyls, cycloalkenyls, aryl, heterocycles, and/or heteroaryls. Unless otherwise specified, preferred aryl groups are those containing 30 or fewer carbon atoms, preferably 6 to 30 carbon atoms, more preferably 6 to 20 carbon atoms, and most preferably 6 to 12 carbon atoms. Especially preferred is an aryl group having 6 carbons, 10 carbons or 12 carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, fluorene, and naphthalene. Additionally, the aryl group is optionally substituted.

The terms “aralkyl” or “arylalkyl” are used interchangeably and refer to an alkyl group that is substituted with an aryl group. Preferred aralkyl groups are those containing 30 or fewer carbon atoms, preferably 6 to 30 carbon atoms. Additionally, the aralkyl group is optionally substituted.

The term “heteroaryl” refers to and includes both monocyclic aromatic groups and polycyclic aromatic groups (ring systems) that comprise at least one heteroatom. The heteroatoms include, but are not limited to O, S, N, P, B, Si, and Se. In many instances, O, S, Se, N or Si are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing 30 or fewer carbon atoms, preferably 3 to 30 carbon atoms, more preferably 3 to 20 carbon atoms, and most preferably 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group is optionally substituted.

The term “heterocyclic group” as used herein contemplates aromatic and non-aromatic cyclic radicals. Hetero-aromatic cyclic radicals also means heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers, such as tetrahydrofuran, tetrahydropyran, and the like. Additionally, the heterocyclic group may be optionally substituted.

The term “arylene” or “arenediyl” as used herein contemplates a substituent of an organic compound that is derived from an aromatic hydrocarbon (arene) that has had a hydrogen atom removed from two ring carbon atoms, such as phenylene. Unless otherwise specified, preferred arylene groups are those containing 30 or fewer carbon atoms, preferably 6 to 30 carbon atoms, more preferably 6 to 20 carbon atoms, and most preferably 6 to 12 carbon atoms. Especially preferred is an arylene group having 6 carbons, 10 carbons or 12 carbons. Additionally, the arylene group is optionally substituted.

The term “cycloalkyl” as used herein contemplates cyclic alkyl radicals. Preferred cycloalkyl groups are those containing 3 to 10 ring carbon atoms and includes cyclopropyl, cyclopentyl, cyclohexyl, adamantyl, and the like. Additionally, the cycloalkyl group may be optionally substituted.

The term “alkenyl” as used herein contemplates both straight and branched chain alkene radicals. Preferred alkenyl groups are those containing two to fifteen carbon atoms. Additionally, the alkenyl group may be optionally substituted.

The term “alkynyl” as used herein contemplates both straight and branched chain alkyne radicals. Preferred alkynyl groups are those containing two to fifteen carbon atoms. Additionally, the alkynyl group may be optionally substituted.

The term “cycloalkyl” as used herein contemplates cyclic alkyl radicals. Preferred cycloalkyl groups are those containing 3 to 10 ring carbon atoms and includes cyclopropyl, cyclopentyl, cyclohexyl, adamantyl, and the like. Additionally, the cycloalkyl group may be optionally substituted.

The terms alkyl, aralkyl, heteroaryl, aryl, cycloalkyl, heteroalkyl, heterocycloalkyl, alkenyl, cycloalkenyl, heteroalkenyl, alkynyl, alkoxy, and heterocyclic group, as used herein, are independently unsubstituted, or independently substituted, with one or more general substituents selected from the group consisting of deuterium, halogen, alkyl, cycloalkyl, heteroalkyl, arylalkyl, alkoxy, aryloxy, amino, cyclic amino, silyl, alkenyl, cycloalkenyl, heteroalkenyl, alkynyl, aryl, heteroaryl, acyl, carbonyl, carboxylic acid, ether, ester, nitrile, isonitrile, sulfanyl, sulfinyl, sulfonyl, phosphino, and combinations thereof.

In many instances, the general substituents are selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof.

In some instances, the preferred general substituents are selected from the group consisting of hydrogen, halogen, trifluoromethyl, cyano, nitro, silyl, and combinations thereof

In yet other instances, the more preferred general substituents are selected from the group consisting of hydrogen, alkyl, aralkyl, heteroaryl and combinations thereof.

The term “acyl” refers to a substituted carbonyl radical (C(O)—Rₛ).

The term “ester” refers to a substituted oxycarbonyl (—O—C(O)—Rₛ or —C(O)—O—Rₛ) radical.

The term “ether” refers to an —ORₛ radical.

The terms “sulfanyl” or “thio-ether” are used interchangeably and refer to a —SRₛ radical.

The term “sulfinyl” refers to a —S(O)—Rₛ radical.

The term “sulfonyl” refers to a —SO₂—Rₛ radical.

The term “phosphino” refers to a —P(Rₛ)₃ radical, wherein each Rₛ can be same or different.

It is to be understood that when a molecular fragment is described as being a substituent or otherwise attached to another moiety, its name may be written as if it were a fragment (e.g., phenyl, phenylene, naphthyl, dibenzofuryl, hydrocarbyl, aromatic linker, arylene) or as if it were the whole molecule (e.g., benzene, naphthalene, dibenzofuran, hydrocarbon, aromatic compound, aromatic hydrocarbon). As used herein, these different ways of designating a substituent or attached fragment are considered to be equivalent.

According to an aspect of the present disclosure, an organic compound of the following formula (1) is disclosed:

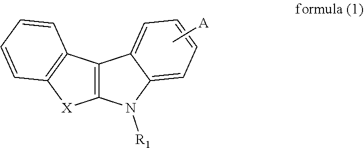

wherein A represents mono to the maximum allowable substitution;
wherein each A comprises at least one chemical group selected from the group consisting of

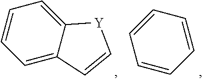

and combinations thereof;
wherein each Y is divalent bridge selected from the group consisting from O, S, CR₇R₈ and NR₉;
wherein X is a divalent bridge selected from the group consisting of O, S and NR₆; and
wherein R₁, R₆, R₇, R₈, and R₉ are independently hydrogen or a substituent selected from the group consisting of alkyl, aralkyl, aralkyl, heteroaryl, deuterium, halide, cycloalkyl, heteroalkyl, arylalkyl, alkoxy, aryloxy, amino, silyl, alkenyl, cycloalkenyl, heteroalkenyl, alkynyl, acyl, carbonyl, carboxylic acids, ester, nitrile, isonitrile, sulfanyl, sulfinyl, sulfonyl, phosphino, and combinations thereof.

In some embodiments, A has the formula (6):

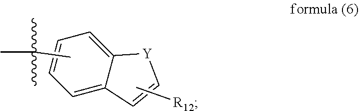

wherein R₁₂ represents mono to the maximum allowable substitution; and
wherein each R₁₂ is hydrogen or a substituent selected from the group consisting of alkyl, aralkyl, aralkyl, heteroaryl, deuterium, halide, cycloalkyl, heteroalkyl, arylalkyl, alkoxy, aryloxy, amino, silyl, alkenyl, cycloalkenyl, heteroalkenyl, alkynyl, acyl, carbonyl, carboxylic acids, ester, nitrile, isonitrile, sulfanyl, sulfinyl, sulfonyl, phosphino, and combinations thereof; and
wherein two or more R₁₂ substituents are optionally joined or fused into a ring.

In some embodiments, A has one of the formula (2) to formula (5):

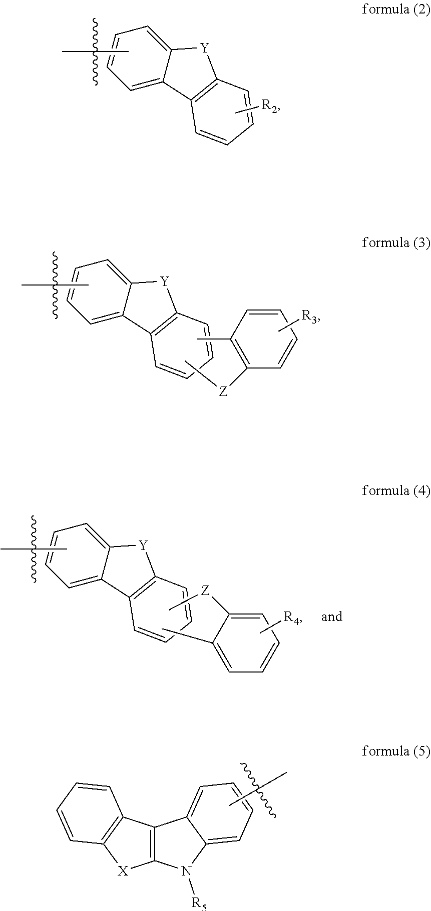

wherein each X is a divalent bridge selected from the group consisting of O, S and NR₆;
each Y is divalent bridge selected from the group consisting from O, S, CR₇R₈ and NR₉;
each Z is divalent bridge selected from the group consisting from O, S, CR₁₀R₁₁ and NR₁₂; and
R₁ to R₁₁ are independently selected from the group consisting of hydrogen, alkyl having 1 to 30 carbon atoms, aryl having 6 to 30 carbon atoms, aralkyl having 6 to 30 carbon atoms, heteroaryl having 6 to 30 carbon atoms, and combinations thereof.

In some embodiments, at least one of R₁, R₅, R₆, R₉ and R₁₂ is selected from the group consisting of triphenylene, carbazole, dibenzothiophene, dibenzofuran, dibenzoselenophene, phenyl, pyridine, pyrimidine, pyrazine, triazine, diazine, benzimidazole, imidazole, quinolone, isoquinolone, quinoazoline, quinoxaline, phenanthrene, biphenyl, terphenyl, o-terphenyl, m-terphenyl, p-terphenyl, and combinations thereof.

In some embodiments, the organic compound has one of the following formula (1-1) to formula (1-4):

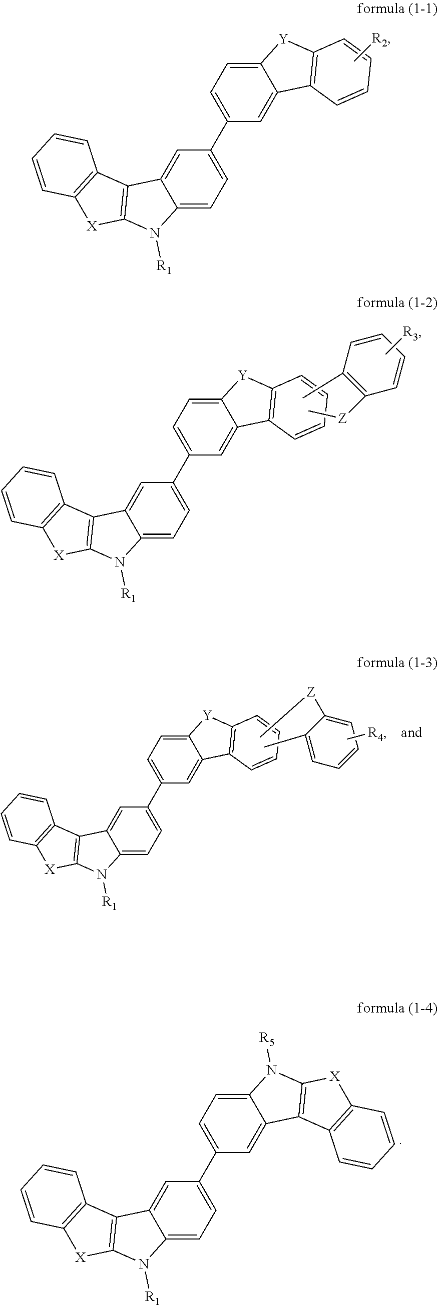

In some embodiments, R₁, R₅, R₆, R₉ and R₁₂ represents one of the following substituents:

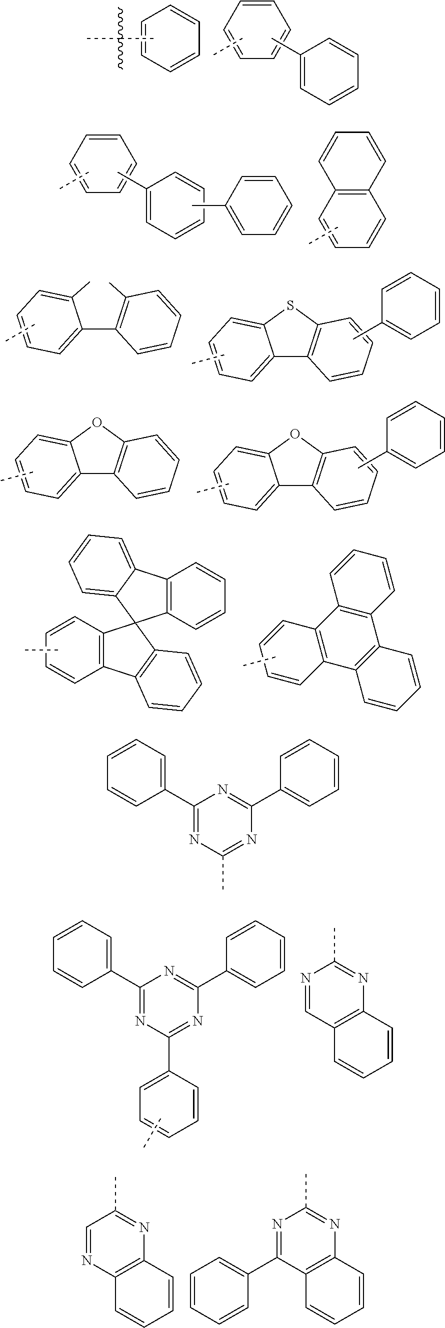

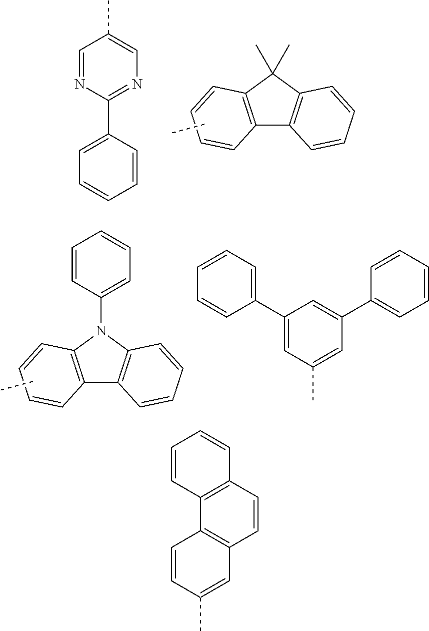

In some embodiments, the organic compound is selected from the group consisting of:

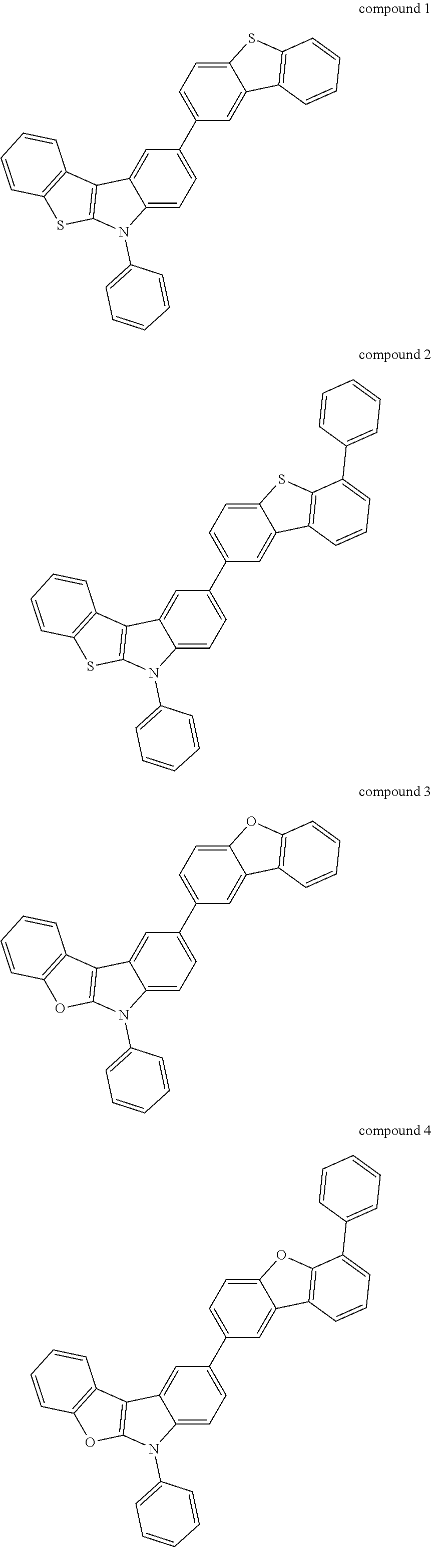 [SMILES: c1ccc(-n2c3ccc(-c4ccc5sc6ccccc6c5c4)cc3c3c4ccccc4sc32)cc1 ; c1ccc(-c2cccc3c2sc2ccc(-c4ccc5c(c4)c4c6ccccc6sc4n5-c4ccccc4)cc23)cc1 ; c1ccc(-n2c3ccc(-c4ccc5oc6ccccc6c5c4)cc3c3c4ccccc4oc32)cc1 ; c1ccc(-c2cccc3c2oc2ccc(-c4ccc5c(c4)c4c6ccccc6oc4n5-c4ccccc4)cc23)cc1]

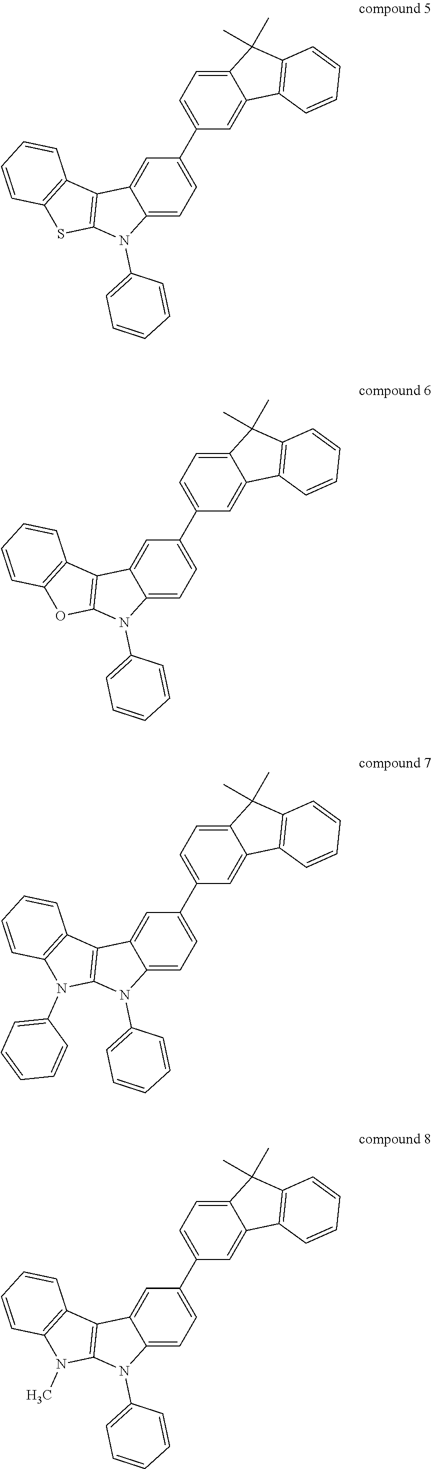 [SMILES: CC1(C)c2ccccc2-c2cc(-c3ccc4c(c3)c3c5ccccc5sc3n4-c3ccccc3)ccc21 ; CC1(C)c2ccccc2-c2cc(-c3ccc4c(c3)c3c5ccccc5oc3n4-c3ccccc3)ccc21 ; CC1(C)c2ccccc2-c2cc(-c3ccc4c(c3)c3c5ccccc5n(-c5ccccc5)c3n4-c3ccccc3)ccc21 ; Cn1c2ccccc2c2c3cc(-c4ccc5c(c4)-c4ccccc4C5(C)C)ccc3n(-c3ccccc3)c21]

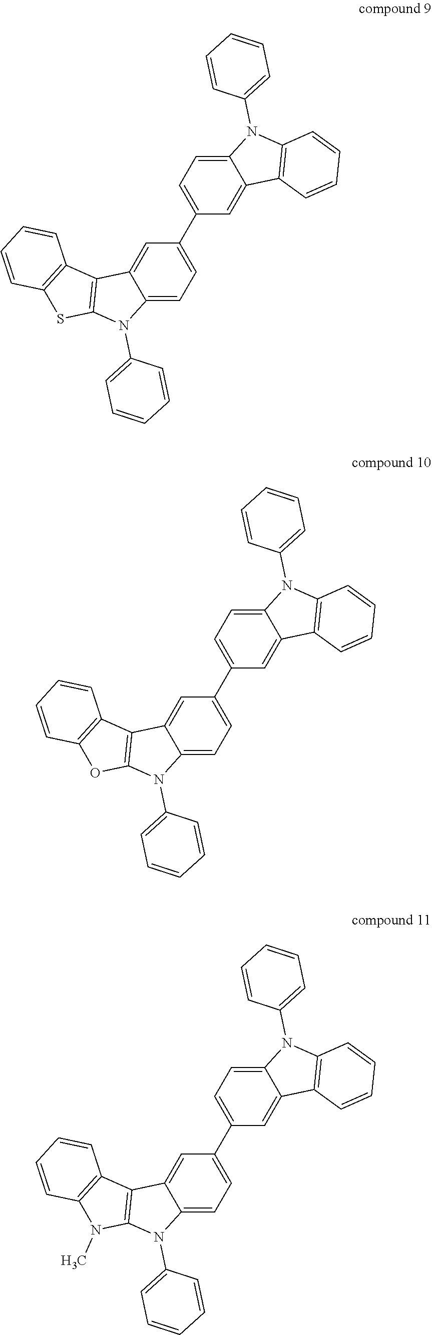 [SMILES: c1ccc(-n2c3ccccc3c3cc(-c4ccc5c(c4)c4c6ccccc6sc4n5-c4ccccc4)ccc32)cc1 ; c1ccc(-n2c3ccccc3c3cc(-c4ccc5c(c4)c4c6ccccc6oc4n5-c4ccccc4)ccc32)cc1 ; Cn1c2ccccc2c2c3cc(-c4ccc5c(c4)c4ccccc4n5-c4ccccc4)ccc3n(-c3ccccc3)c21]

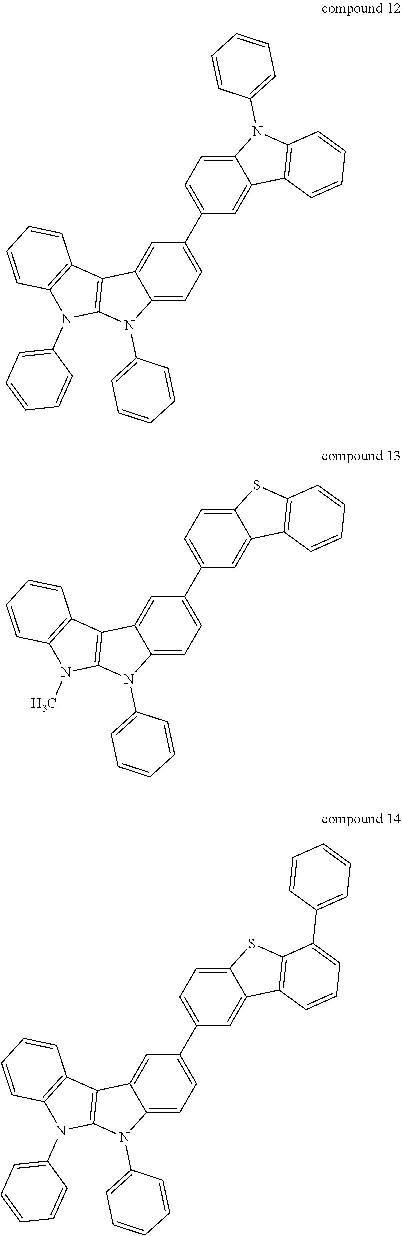 [SMILES: c1ccc(-n2c3ccccc3c3cc(-c4ccc5c(c4)c4c6ccccc6n(-c6ccccc6)c4n5-c4ccccc4)ccc32)cc1 ; Cn1c2ccccc2c2c3cc(-c4ccc5sc6ccccc6c5c4)ccc3n(-c3ccccc3)c21 ; c1ccc(-c2cccc3c2sc2ccc(-c4ccc5c(c4)c4c6ccccc6n(-c6ccccc6)c4n5-c4ccccc4)cc23)cc1]

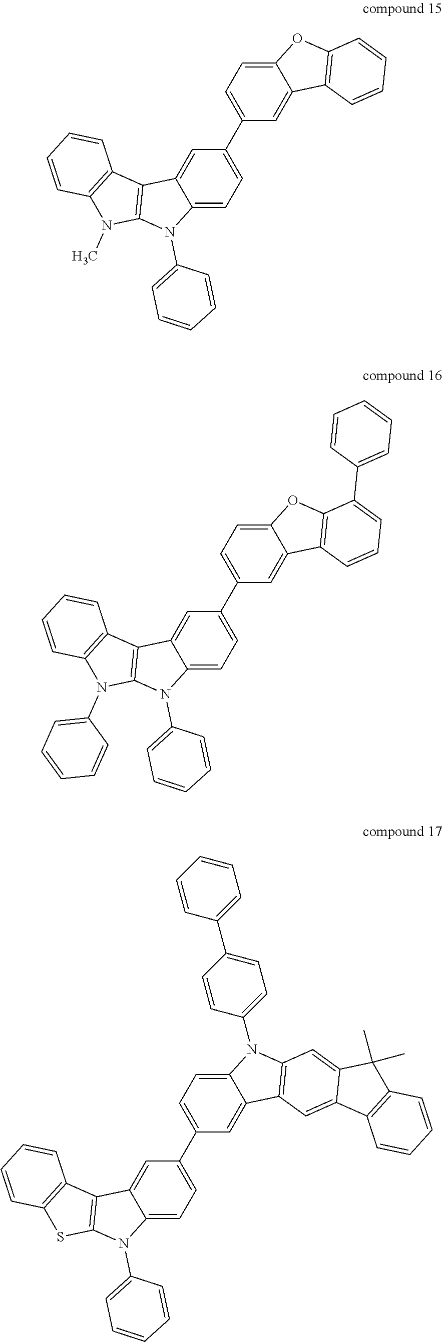 [SMILES: Cn1c2ccccc2c2c3cc(-c4ccc5oc6ccccc6c5c4)ccc3n(-c3ccccc3)c21 ; c1ccc(-c2cccc3c2oc2ccc(-c4ccc5c(c4)c4c6ccccc6n(-c6ccccc6)c4n5-c4ccccc4)cc23)cc1 ; CC1(C)c2ccccc2-c2cc3c4cc(-c5ccc6c(c5)c5c7ccccc7sc5n6-c5ccccc5)ccc4n(-c4ccc(-c5ccccc5)cc4)c3cc21]

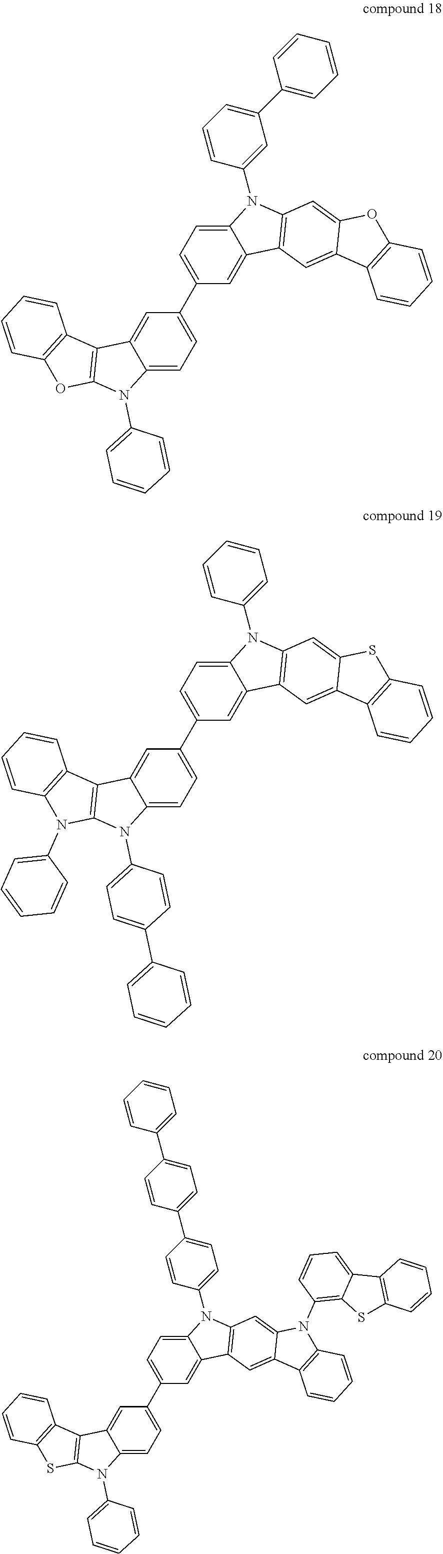 [SMILES: c1ccc(-c2cccc(-n3c4ccc(-c5ccc6c(c5)c5c7ccccc7oc5n6-c5ccccc5)cc4c4cc5c(cc43)oc3ccccc35)c2)cc1 ; c1ccc(-c2ccc(-n3c4ccc(-c5ccc6c(c5)c5cc7c(cc5n6-c5ccccc5)sc5ccccc57)cc4c4c5ccccc5n(-c5ccccc5)c43)cc2)cc1 ; c1ccc(-c2ccc(-c3ccc(-n4c5ccc(-c6ccc7c(c6)c6c8ccccc8sc6n7-c6ccccc6)cc5c5cc6c7ccccc7n(-c7cccc8c7sc7ccccc78)c6cc54)cc3)cc2)cc1]

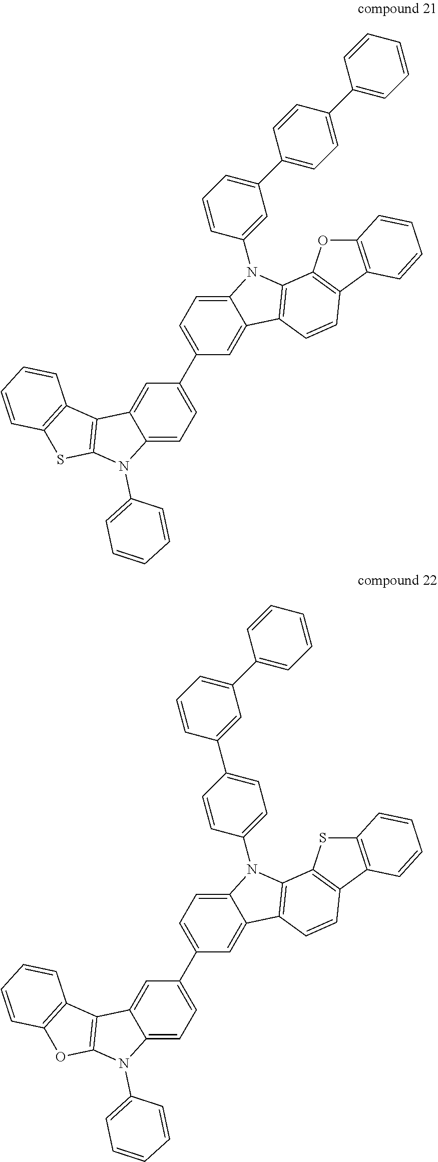 [SMILES: c1ccc(-c2ccc(-c3cccc(-n4c5ccc(-c6ccc7c(c6)c6c8ccccc8sc6n7-c6ccccc6)cc5c5ccc6c7ccccc7oc6c54)c3)cc2)cc1 ; c1ccc(-c2cccc(-c3ccc(-n4c5ccc(-c6ccc7c(c6)c6c8ccccc8oc6n7-c6ccccc6)cc5c5ccc6c7ccccc7sc6c54)cc3)c2)cc1]

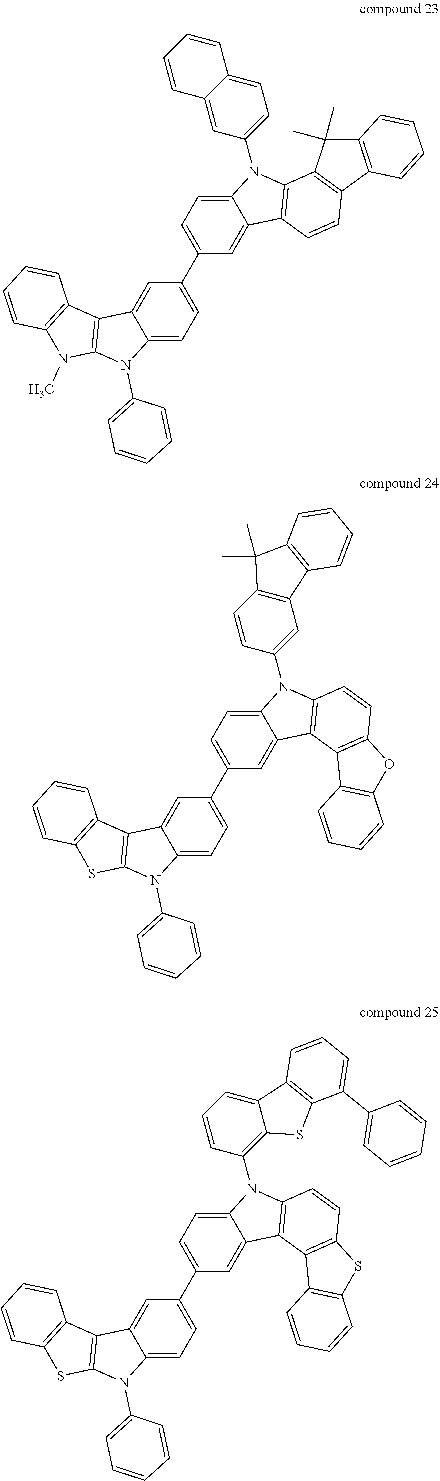 [SMILES: Cn1c2ccccc2c2c3cc(-c4ccc5c(c4)c4ccc6c(c4n5-c4ccc5ccccc5c4)C(C)(C)c4ccccc4-6)ccc3n(-c3ccccc3)c21 ; CC1(C)c2ccccc2-c2cc(-n3c4ccc(-c5ccc6c(c5)c5c7ccccc7sc5n6-c5ccccc5)cc4c4c5c(ccc43)oc3ccccc35)ccc21 ; c1ccc(-c2cccc3c2sc2c(-n4c5ccc(-c6ccc7c(c6)c6c8ccccc8sc6n7-c6ccccc6)cc5c5c6c(ccc54)sc4ccccc46)cccc23)cc1]

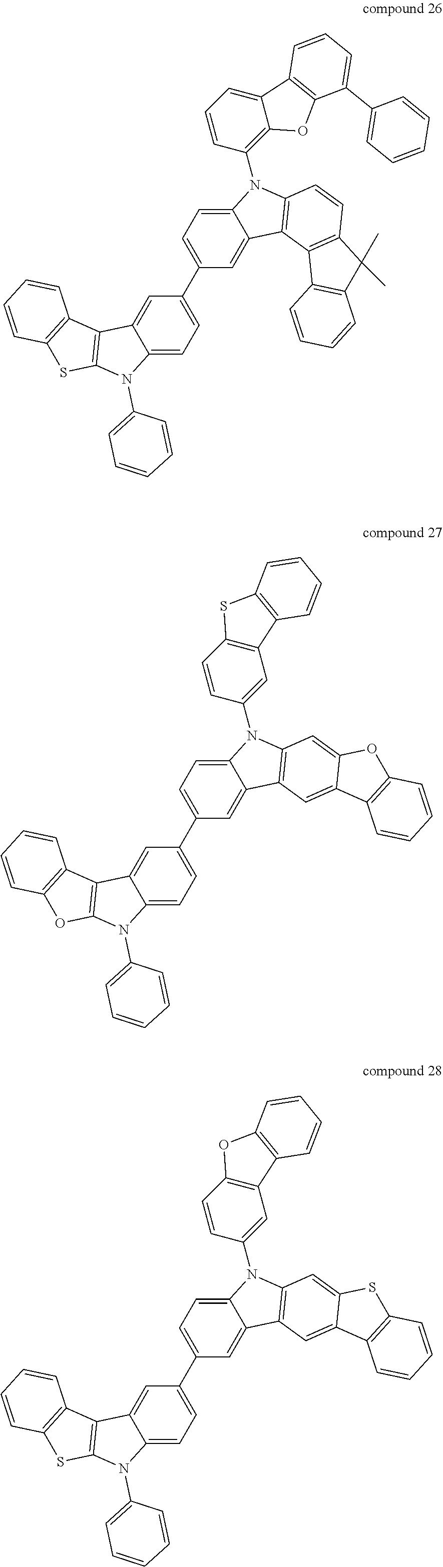 [SMILES: CC1(C)c2ccccc2-c2c1ccc1c2c2cc(-c3ccc4c(c3)c3c5ccccc5sc3n4-c3ccccc3)ccc2n1-c1cccc2c1oc1c(-c3ccccc3)cccc12 ; c1ccc(-n2c3ccc(-c4ccc5c(c4)c4cc6c(cc4n5-c4ccc5sc7ccccc7c5c4)oc4ccccc46)cc3c3c4ccccc4oc32)cc1 ; c1ccc(-n2c3ccc(-c4ccc5c(c4)c4cc6c(cc4n5-c4ccc5oc7ccccc7c5c4)sc4ccccc46)cc3c3c4ccccc4sc32)cc1]

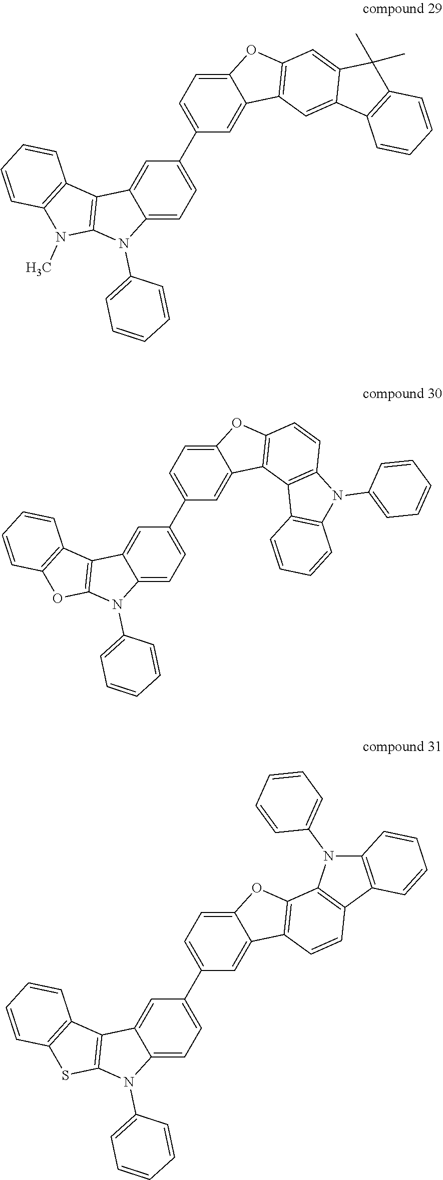 [SMILES: Cn1c2ccccc2c2c3cc(-c4ccc5oc6cc7c(cc6c5c4)-c4ccccc4C7(C)C)ccc3n(-c3ccccc3)c21 ; c1ccc(-n2c3ccccc3c3c4c(ccc32)oc2ccc(-c3ccc5c(c3)c3c6ccccc6oc3n5-c3ccccc3)cc24)cc1 ; c1ccc(-n2c3ccc(-c4ccc5oc6c(ccc7c8ccccc8n(-c8ccccc8)c76)c5c4)cc3c3c4ccccc4sc32)cc1]

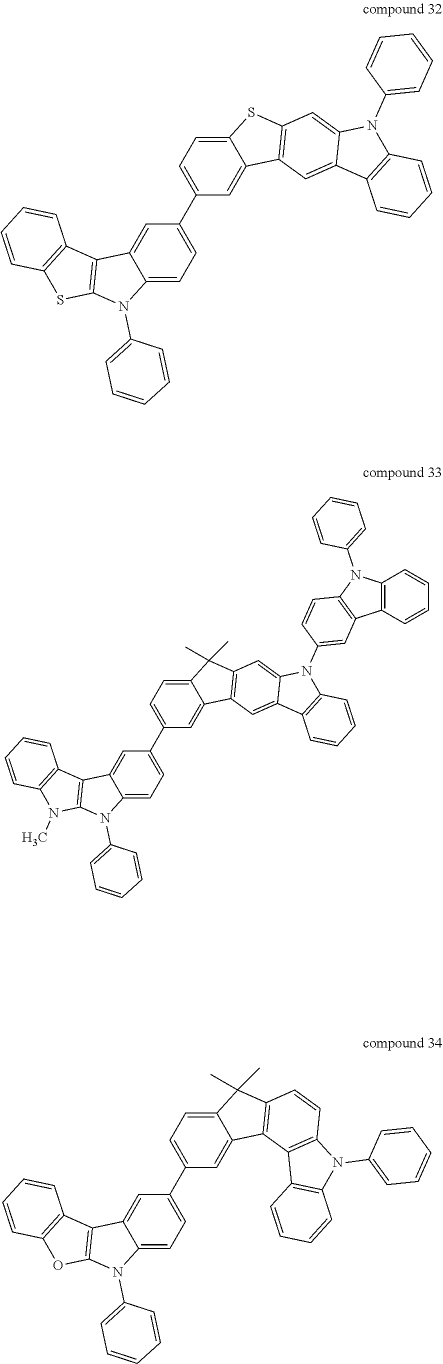 [SMILES: c1ccc(-n2c3ccccc3c3cc4c(cc32)sc2ccc(-c3ccc5c(c3)c3c6ccccc6sc3n5-c3ccccc3)cc24)cc1 ; Cn1c2ccccc2c2c3cc(-c4ccc5c(c4)-c4cc6c7ccccc7n(-c7ccc8c(c7)c7ccccc7n8-c7ccccc7)c6cc4C5(C)C)ccc3n(-c3ccccc3)c21 ; CC1(C)c2ccc(-c3ccc4c(c3)c3c5ccccc5oc3n4-c3ccccc3)cc2-c2c1ccc1c2c2ccccc2n1-c1ccccc1]

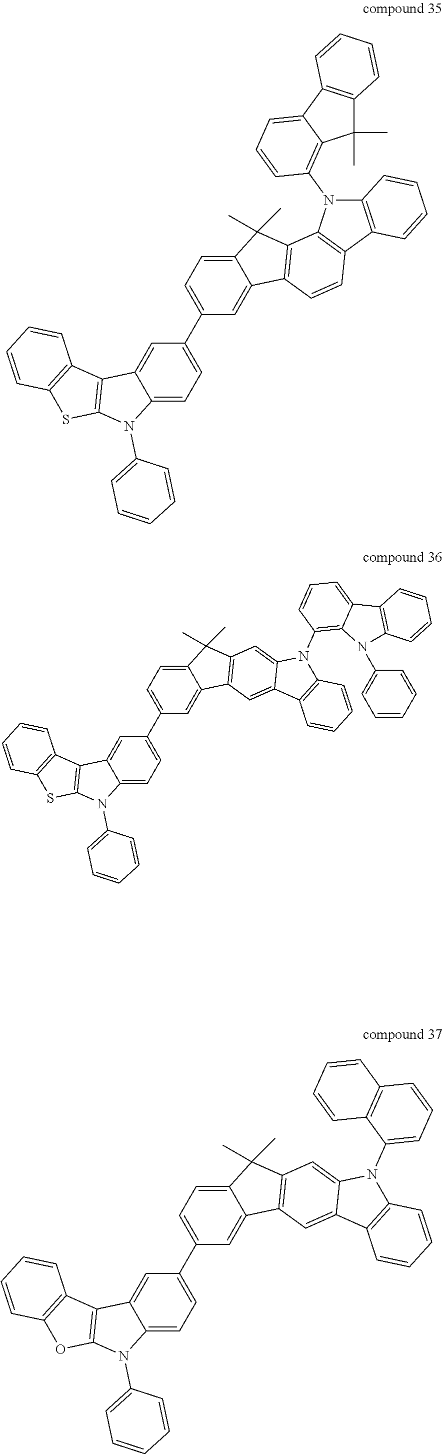 [SMILES: CC1(C)c2ccccc2-c2cccc(-n3c4ccccc4c4ccc5c(c43)C(C)(C)c3ccc(-c4ccc6c(c4)c4c7ccccc7sc4n6-c4ccccc4)cc3-5)c21 ; CC1(C)c2ccc(-c3ccc4c(c3)c3c5ccccc5sc3n4-c3ccccc3)cc2-c2cc3c4ccccc4n(-c4cccc5c6ccccc6n(-c6ccccc6)c45)c3cc21 ; CC1(C)c2ccc(-c3ccc4c(c3)c3c5ccccc5oc3n4-c3ccccc3)cc2-c2cc3c4ccccc4n(-c4cccc5ccccc45)c3cc21]

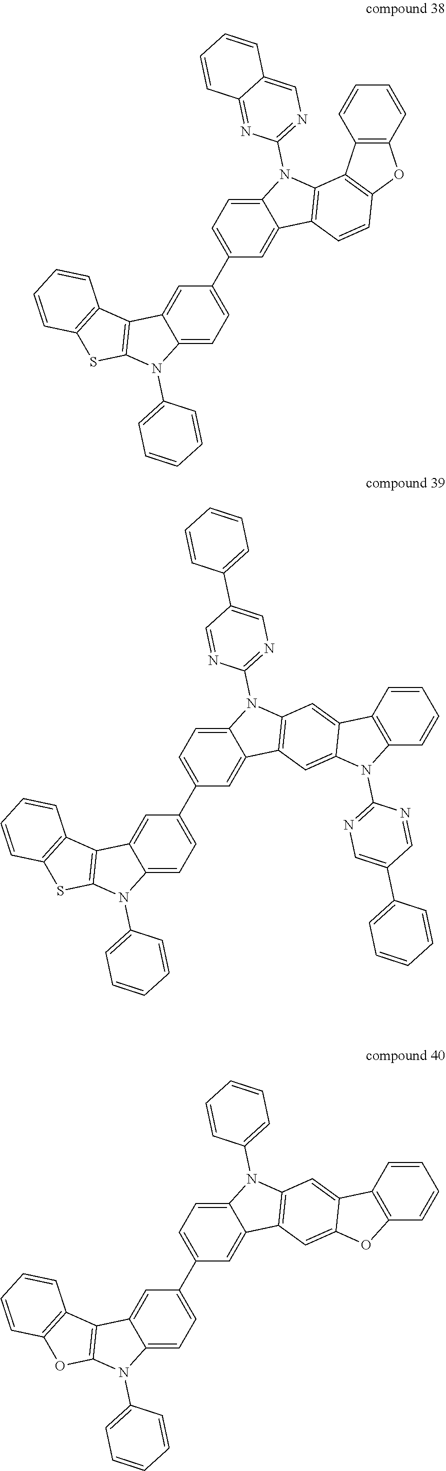 [SMILES: c1ccc(-n2c3ccc(-c4ccc5c(c4)c4ccc6oc7ccccc7c6c4n5-c4ncc5ccccc5n4)cc3c3c4ccccc4sc32)cc1 ; c1ccc(-c2cnc(-n3c4ccccc4c4cc5c(cc43)c3cc(-c4ccc6c(c4)c4c7ccccc7sc4n6-c4ccccc4)ccc3n5-c3ncc(-c4ccccc4)cn3)nc2)cc1 ; c1ccc(-n2c3ccc(-c4ccc5c(c4)c4c6ccccc6oc4n5-c4ccccc4)cc3c3cc4oc5ccccc5c4cc32)cc1]

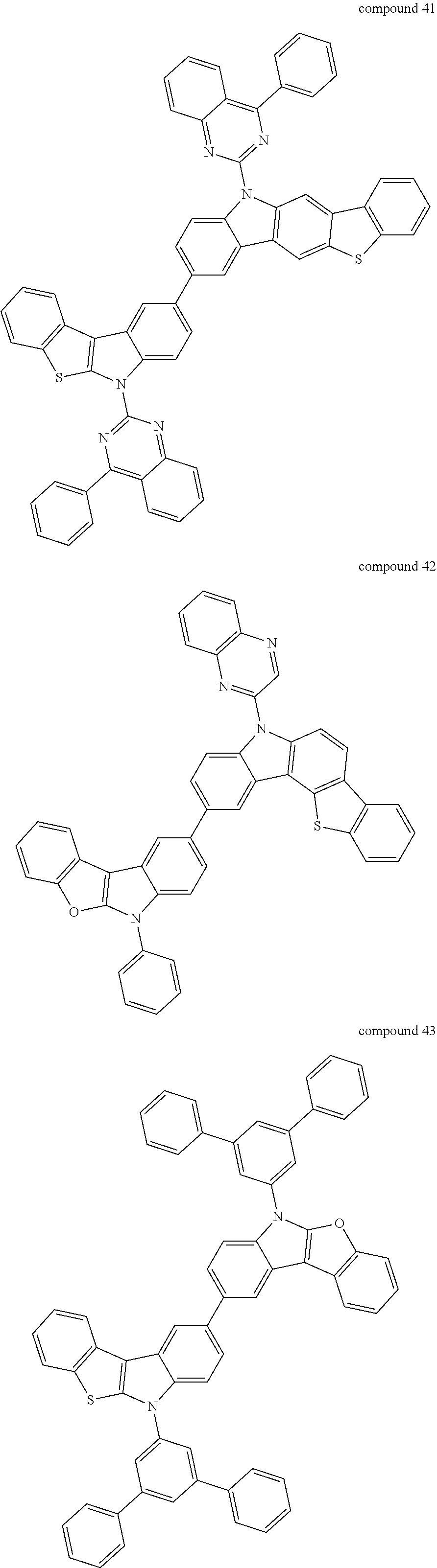 [SMILES: c1ccc(-c2nc(-n3c4ccc(-c5ccc6c(c5)c5c7ccccc7sc5n6-c5nc(-c6ccccc6)c6ccccc6n5)cc4c4cc5sc6ccccc6c5cc43)nc3ccccc23)cc1 ; c1ccc(-n2c3ccc(-c4ccc5c(c4)c4c6sc7ccccc7c6ccc4n5-c4cnc5ccccc5n4)cc3c3c4ccccc4oc32)cc1 ; c1ccc(-c2cc(-c3ccccc3)cc(-n3c4ccc(-c5ccc6c(c5)c5c7ccccc7sc5n6-c5cc(-c6ccccc6)cc(-c6ccccc6)c5)cc4c4c5ccccc5oc43)c2)cc1]

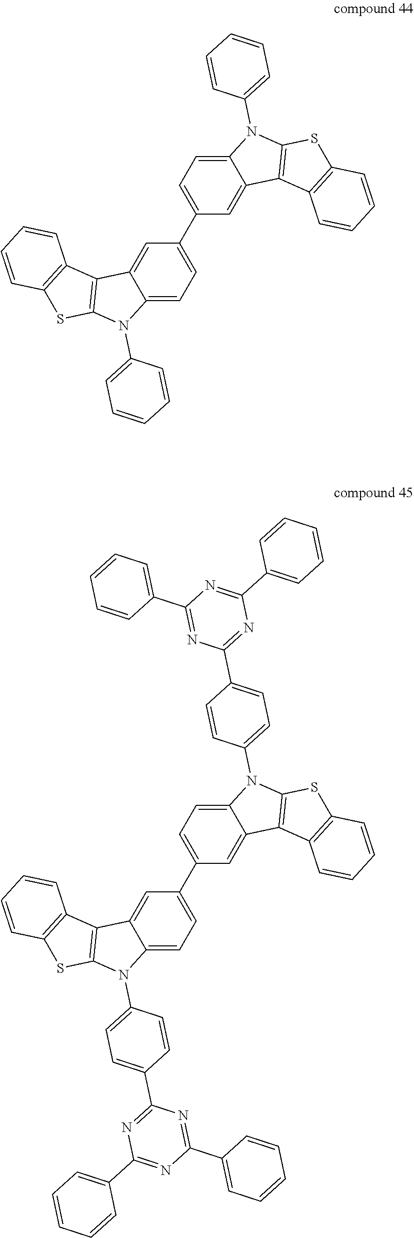 [SMILES: c1ccc(-n2c3ccc(-c4ccc5c(c4)c4c6ccccc6sc4n5-c4ccccc4)cc3c3c4ccccc4sc32)cc1 ; c1ccc(-c2nc(-c3ccccc3)nc(-c3ccc(-n4c5ccc(-c6ccc7c(c6)c6c8ccccc8sc6n7-c6ccc(-c7nc(-c8ccccc8)nc(-c8ccccc8)n7)cc6)cc5c5c6ccccc6sc54)cc3)n2)cc1]

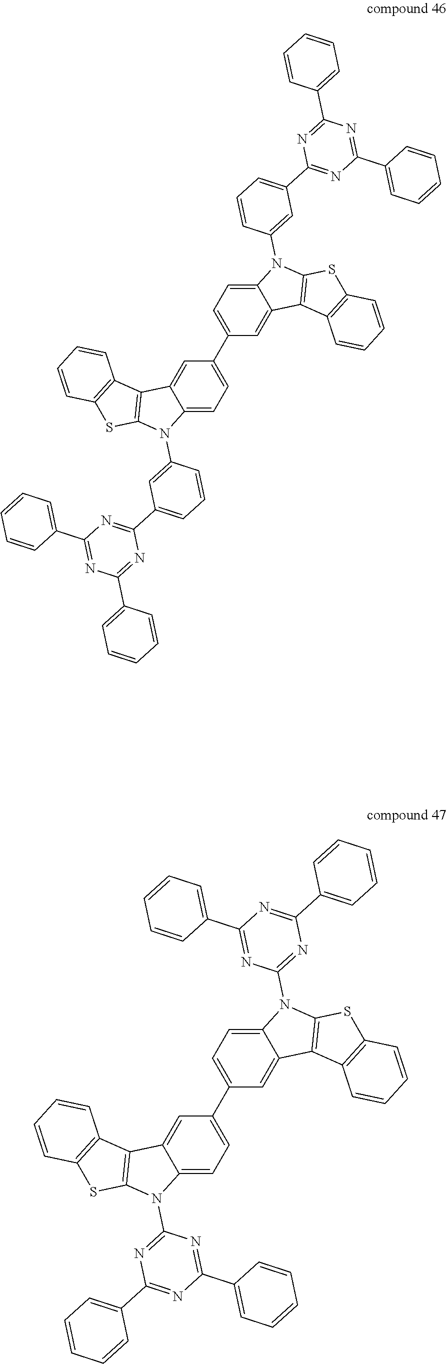 [SMILES: c1ccc(-c2nc(-c3ccccc3)nc(-c3cccc(-n4c5ccc(-c6ccc7c(c6)c6c8ccccc8sc6n7-c6cccc(-c7nc(-c8ccccc8)nc(-c8ccccc8)n7)c6)cc5c5c6ccccc6sc54)c3)n2)cc1 ; c1ccc(-c2nc(-c3ccccc3)nc(-n3c4ccc(-c5ccc6c(c5)c5c7ccccc7sc5n6-c5nc(-c6ccccc6)nc(-c6ccccc6)n5)cc4c4c5ccccc5sc43)n2)cc1]

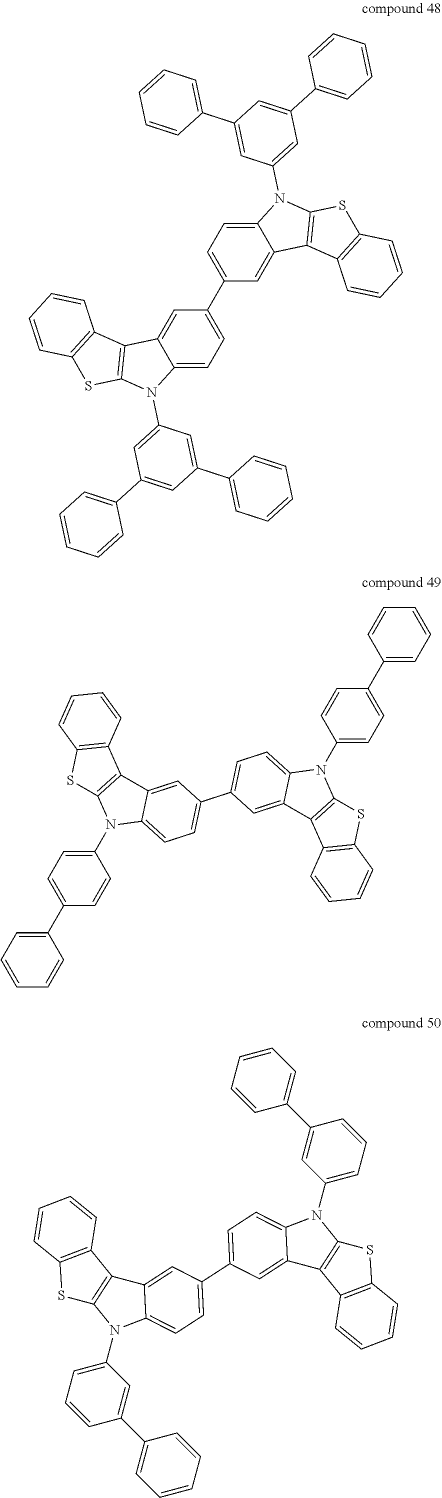 [SMILES: c1ccc(-c2cc(-c3ccccc3)cc(-n3c4ccc(-c5ccc6c(c5)c5c7ccccc7sc5n6-c5cc(-c6ccccc6)cc(-c6ccccc6)c5)cc4c4c5ccccc5sc43)c2)cc1 ; c1ccc(-c2ccc(-n3c4ccc(-c5ccc6c(c5)c5c7ccccc7sc5n6-c5ccc(-c6ccccc6)cc5)cc4c4c5ccccc5sc43)cc2)cc1 ; c1ccc(-c2cccc(-n3c4ccc(-c5ccc6c(c5)c5c7ccccc7sc5n6-c5cccc(-c6ccccc6)c5)cc4c4c5ccccc5sc43)c2)cc1]

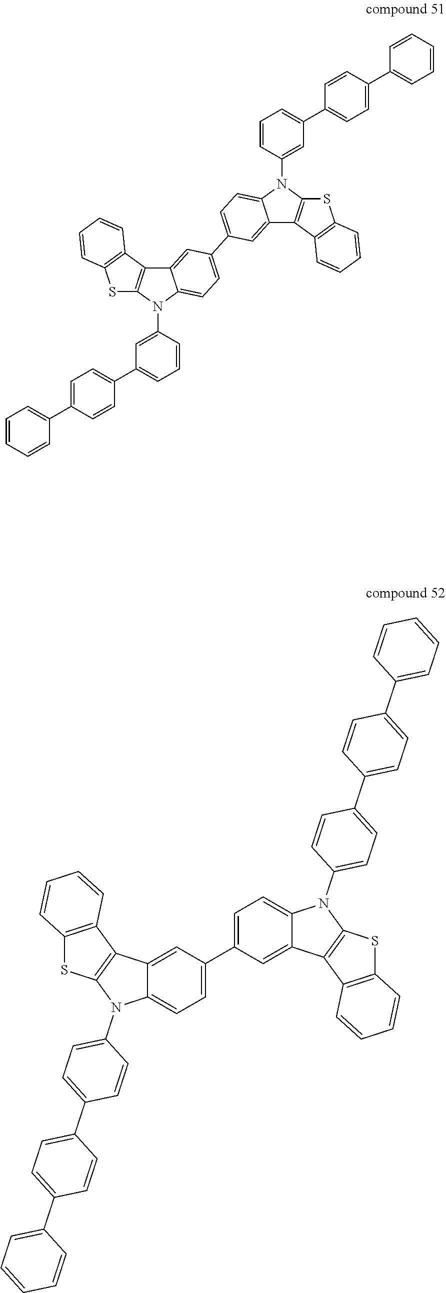 [SMILES: c1ccc(-c2ccc(-c3cccc(-n4c5ccc(-c6ccc7c(c6)c6c8ccccc8sc6n7-c6cccc(-c7ccc(-c8ccccc8)cc7)c6)cc5c5c6ccccc6sc54)c3)cc2)cc1 ; c1ccc(-c2ccc(-c3ccc(-n4c5ccc(-c6ccc7c(c6)c6c8ccccc8sc6n7-c6ccc(-c7ccc(-c8ccccc8)cc7)cc6)cc5c5c6ccccc6sc54)cc3)cc2)cc1]

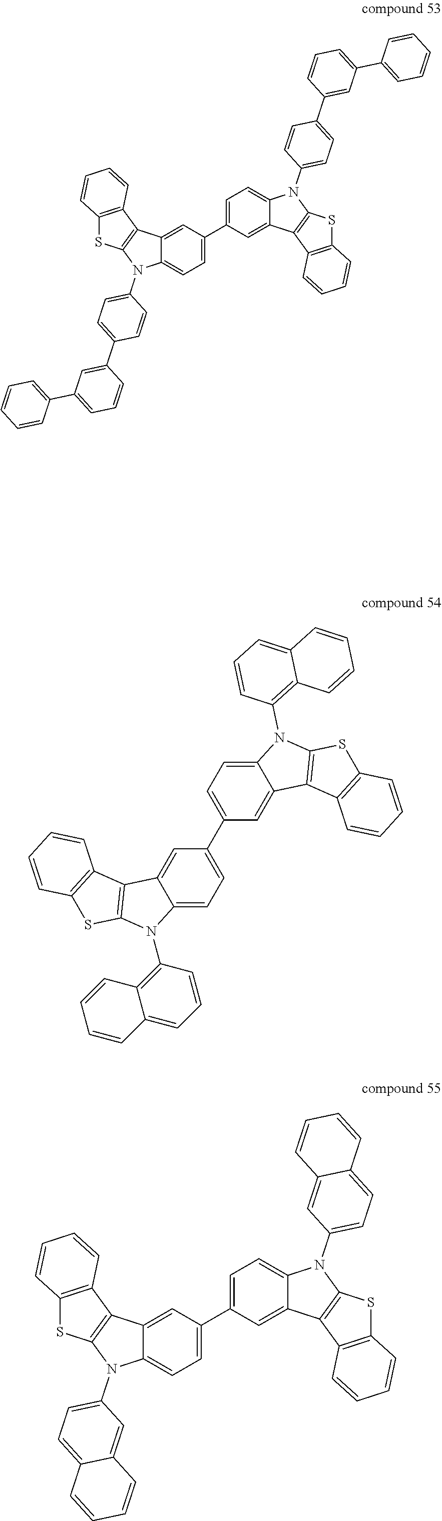 [SMILES: c1ccc(-c2cccc(-c3ccc(-n4c5ccc(-c6ccc7c(c6)c6c8ccccc8sc6n7-c6ccc(-c7cccc(-c8ccccc8)c7)cc6)cc5c5c6ccccc6sc54)cc3)c2)cc1 ; c1ccc2c(-n3c4ccc(-c5ccc6c(c5)c5c7ccccc7sc5n6-c5cccc6ccccc56)cc4c4c5ccccc5sc43)cccc2c1 ; c1ccc2cc(-n3c4ccc(-c5ccc6c(c5)c5c7ccccc7sc5n6-c5ccc6ccccc6c5)cc4c4c5ccccc5sc43)ccc2c1]

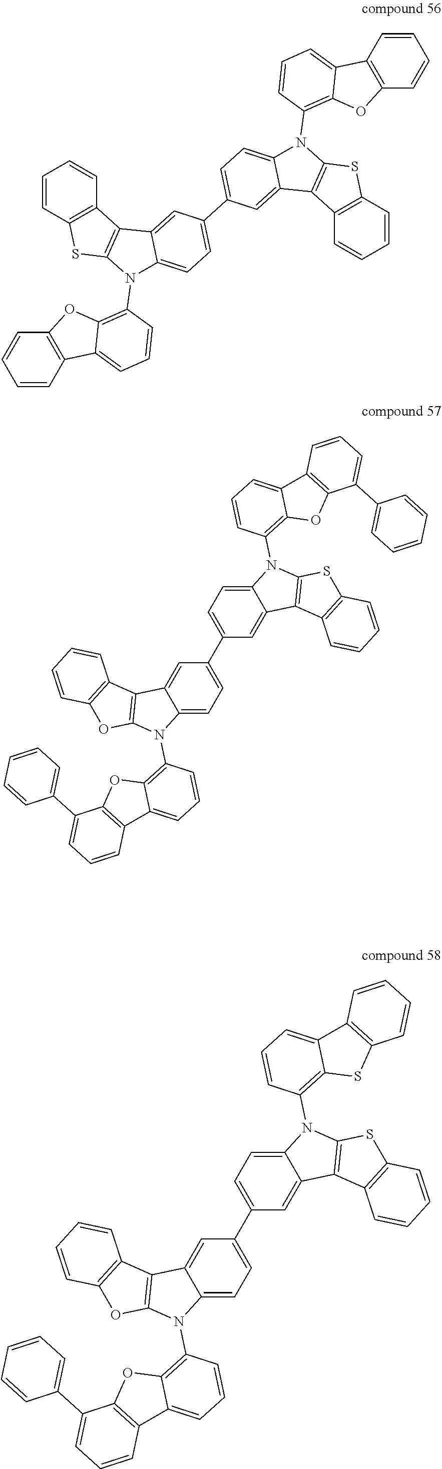 [SMILES: c1ccc2c(c1)oc1c(-n3c4ccc(-c5ccc6c(c5)c5c7ccccc7sc5n6-c5cccc6c5oc5ccccc56)cc4c4c5ccccc5sc43)cccc12 ; c1ccc(-c2cccc3c2oc2c(-n4c5ccc(-c6ccc7c(c6)c6c8ccccc8sc6n7-c6cccc7c6oc6c(-c8ccccc8)cccc67)cc5c5c6ccccc6oc54)cccc23)cc1 ; c1ccc(-c2cccc3c2oc2c(-n4c5ccc(-c6ccc7c(c6)c6c8ccccc8sc6n7-c6cccc7c6sc6ccccc67)cc5c5c6ccccc6oc54)cccc23)cc1]

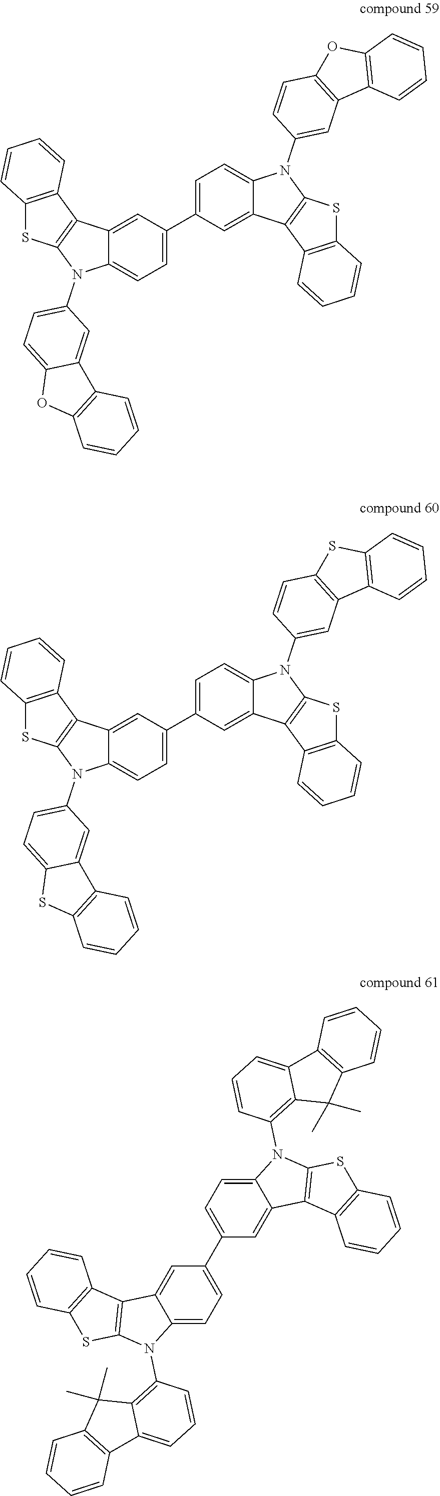 [SMILES: c1ccc2c(c1)oc1ccc(-n3c4ccc(-c5ccc6c(c5)c5c7ccccc7sc5n6-c5ccc6oc7ccccc7c6c5)cc4c4c5ccccc5sc43)cc12 ; c1ccc2c(c1)sc1ccc(-n3c4ccc(-c5ccc6c(c5)c5c7ccccc7sc5n6-c5ccc6sc7ccccc7c6c5)cc4c4c5ccccc5sc43)cc12 ; CC1(C)c2ccccc2-c2cccc(-n3c4ccc(-c5ccc6c(c5)c5c7ccccc7sc5n6-c5cccc6c5C(C)(C)c5ccccc5-6)cc4c4c5ccccc5sc43)c21]

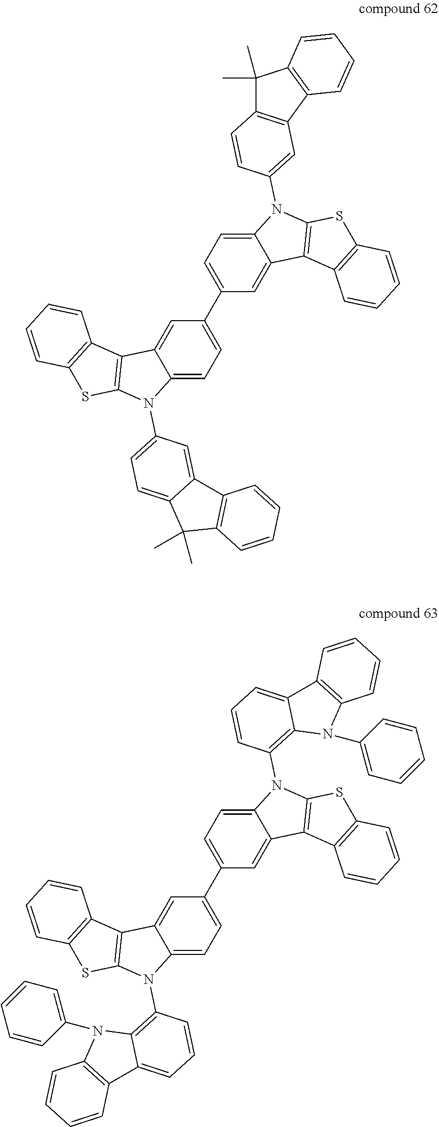 [SMILES: CC1(C)c2ccccc2-c2cc(-n3c4ccc(-c5ccc6c(c5)c5c7ccccc7sc5n6-c5ccc6c(c5)-c5ccccc5C6(C)C)cc4c4c5ccccc5sc43)ccc21 ; c1ccc(-n2c3ccccc3c3cccc(-n4c5ccc(-c6ccc7c(c6)c6c8ccccc8sc6n7-c6cccc7c8ccccc8n(-c8ccccc8)c67)cc5c5c6ccccc6sc54)c32)cc1]

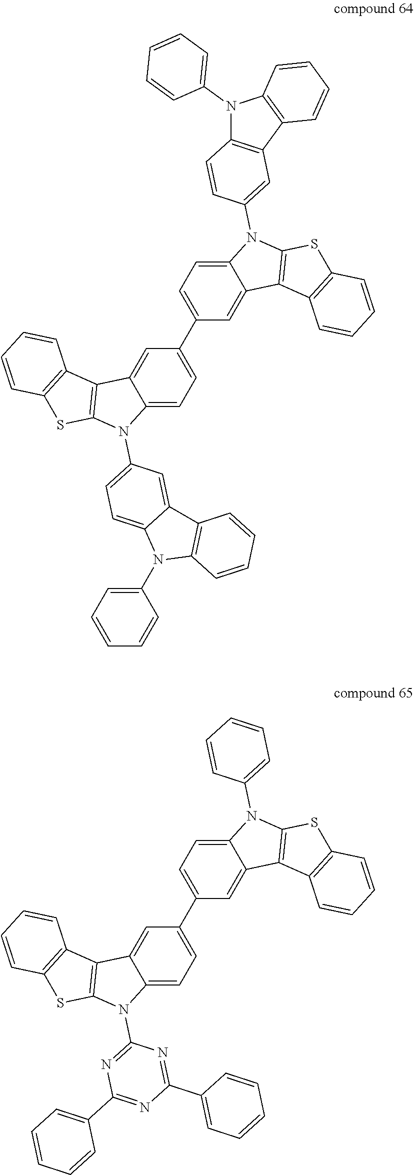 [SMILES: c1ccc(-n2c3ccccc3c3cc(-n4c5ccc(-c6ccc7c(c6)c6c8ccccc8sc6n7-c6ccc7c(c6)c6ccccc6n7-c6ccccc6)cc5c5c6ccccc6sc54)ccc32)cc1 ; c1ccc(-c2nc(-c3ccccc3)nc(-n3c4ccc(-c5ccc6c(c5)c5c7ccccc7sc5n6-c5ccccc5)cc4c4c5ccccc5sc43)n2)cc1]

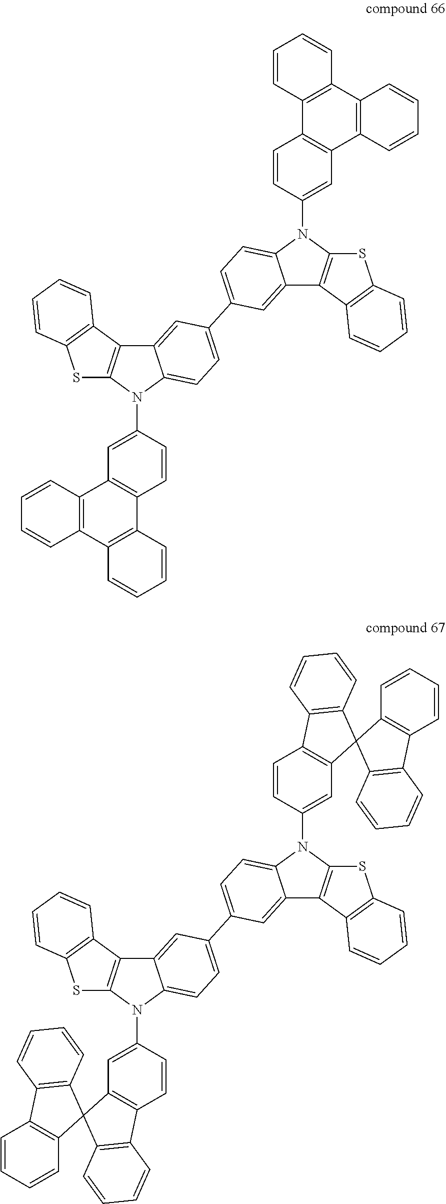 [SMILES: c1ccc2c(c1)sc1c2c2cc(-c3ccc4c(c3)c3c5ccccc5sc3n4-c3ccc4c5ccccc5c5ccccc5c4c3)ccc2n1-c1ccc2c3ccccc3c3ccccc3c2c1 ; c1ccc2c(c1)-c1ccccc1C21c2ccccc2-c2ccc(-n3c4ccc(-c5ccc6c(c5)c5c7ccccc7sc5n6-c5ccc6c(c5)C5(c7ccccc7-c7ccccc75)c5ccccc5-6)cc4c4c5ccccc5sc43)cc21]

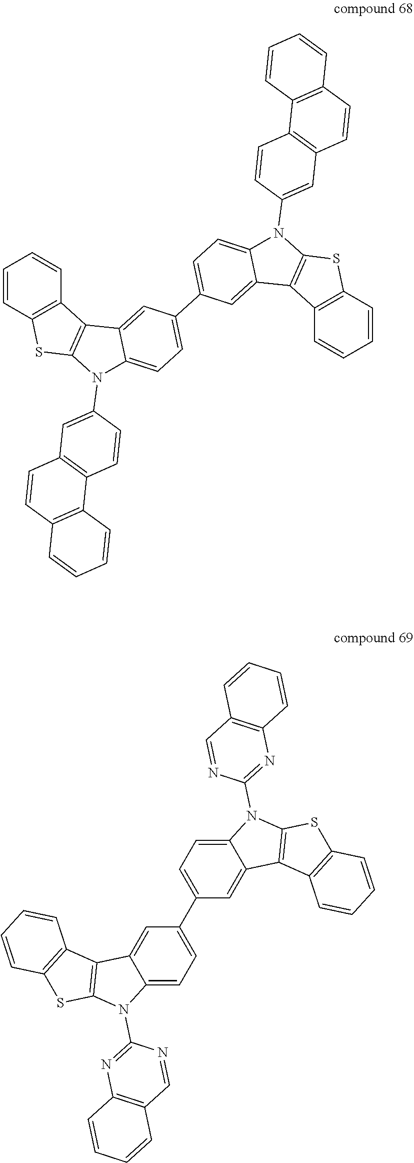 [SMILES: c1ccc2c(c1)ccc1cc(-n3c4ccc(-c5ccc6c(c5)c5c7ccccc7sc5n6-c5ccc6c(ccc7ccccc76)c5)cc4c4c5ccccc5sc43)ccc12 ; c1ccc2nc(-n3c4ccc(-c5ccc6c(c5)c5c7ccccc7sc5n6-c5ncc6ccccc6n5)cc4c4c5ccccc5sc43)ncc2c1]

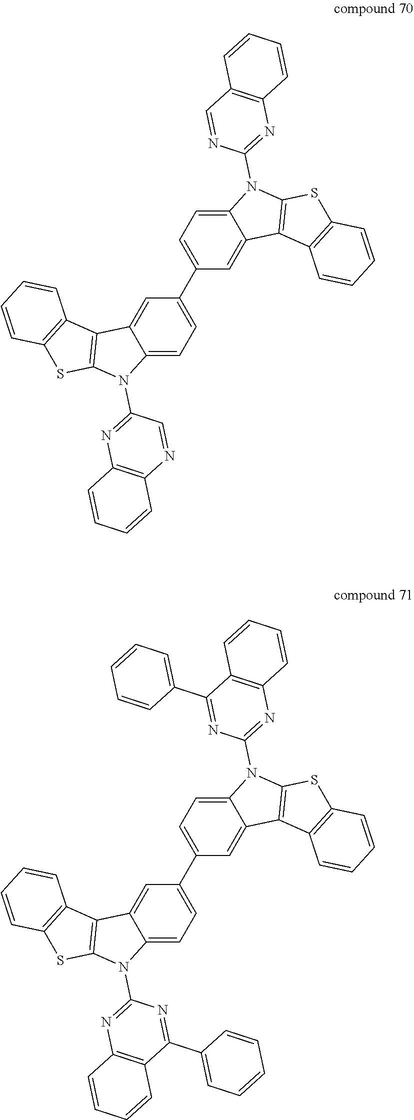 [SMILES: c1ccc2nc(-n3c4ccc(-c5ccc6c(c5)c5c7ccccc7sc5n6-c5cnc6ccccc6n5)cc4c4c5ccccc5sc43)ncc2c1 ; c1ccc(-c2nc(-n3c4ccc(-c5ccc6c(c5)c5c7ccccc7sc5n6-c5nc(-c6ccccc6)c6ccccc6n5)cc4c4c5ccccc5sc43)nc3ccccc23)cc1]

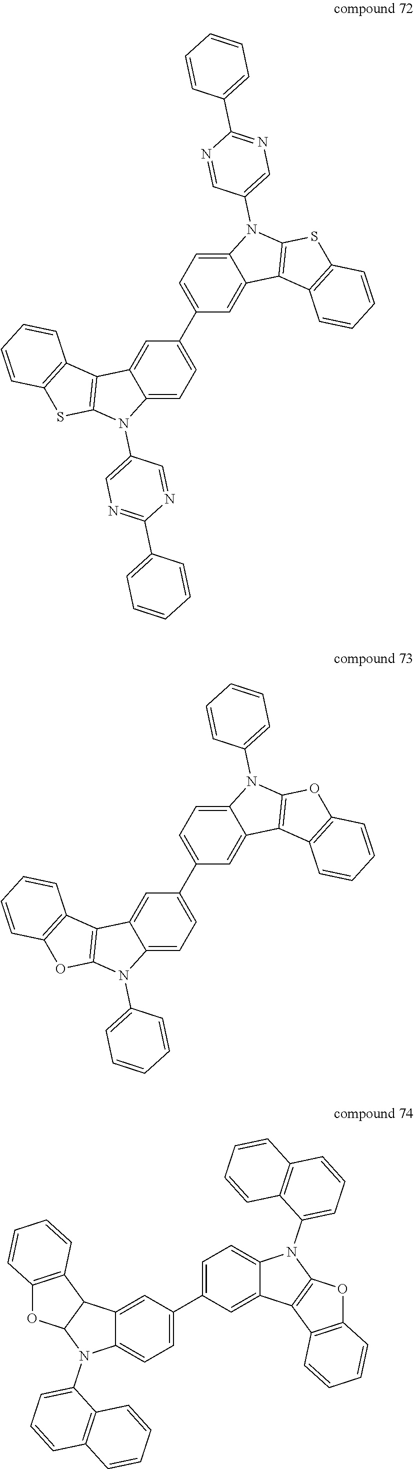 [SMILES: c1ccc(-c2ncc(-n3c4ccc(-c5ccc6c(c5)c5c7ccccc7sc5n6-c5cnc(-c6ccccc6)nc5)cc4c4c5ccccc5sc43)cn2)cc1 ; c1ccc(-n2c3ccc(-c4ccc5c(c4)c4c6ccccc6oc4n5-c4ccccc4)cc3c3c4ccccc4oc32)cc1 ; c1ccc2c(c1)OC1C2c2cc(-c3ccc4c(c3)c3c5ccccc5oc3n4-c3cccc4ccccc34)ccc2N1c1cccc2ccccc12]

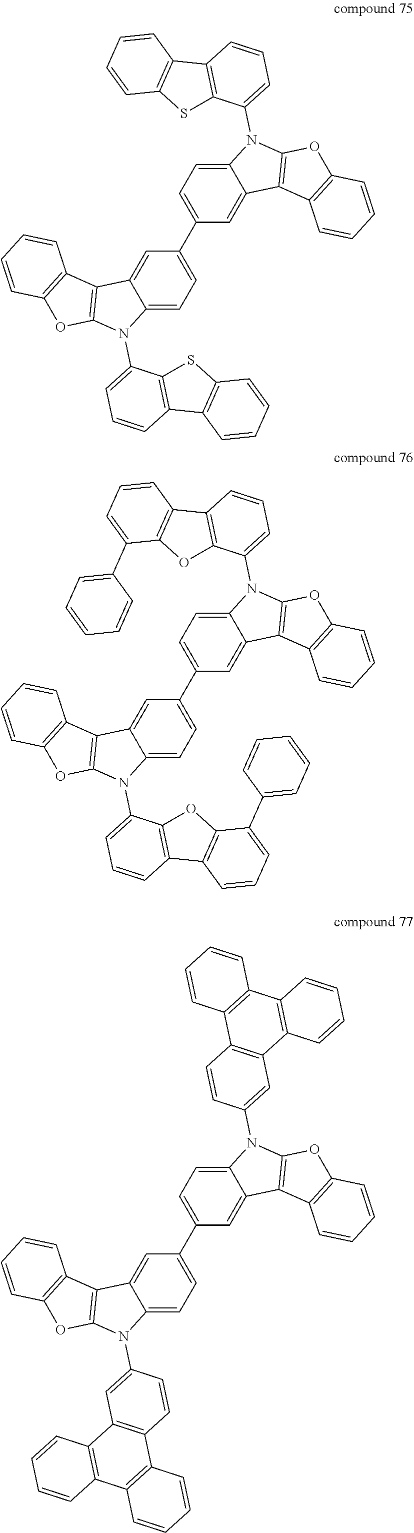 [SMILES: c1ccc2c(c1)oc1c2c2cc(-c3ccc4c(c3)c3c5ccccc5oc3n4-c3cccc4c3sc3ccccc34)ccc2n1-c1cccc2c1sc1ccccc12 ; c1ccc(-c2cccc3c2oc2c(-n4c5ccc(-c6ccc7c(c6)c6c8ccccc8oc6n7-c6cccc7c6oc6c(-c8ccccc8)cccc67)cc5c5c6ccccc6oc54)cccc23)cc1 ; c1ccc2c(c1)oc1c2c2cc(-c3ccc4c(c3)c3c5ccccc5oc3n4-c3ccc4c5ccccc5c5ccccc5c4c3)ccc2n1-c1ccc2c3ccccc3c3ccccc3c2c1]

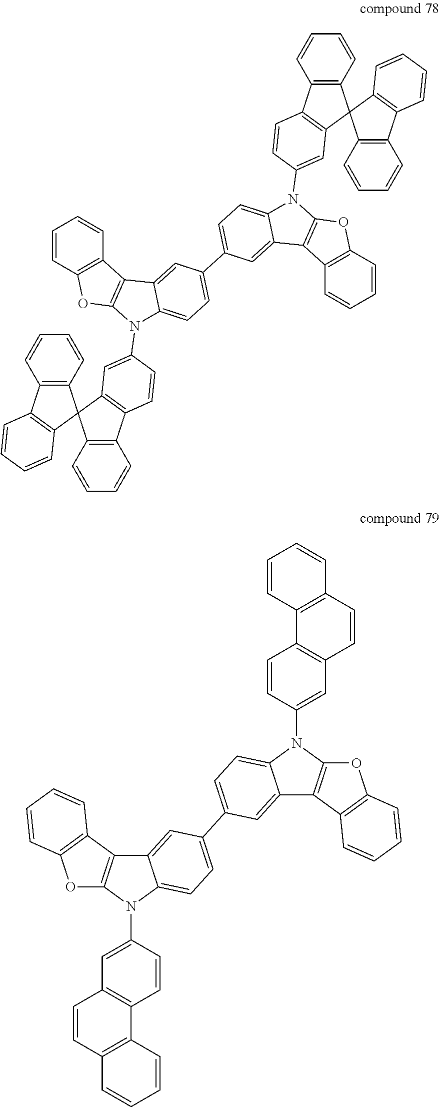 [SMILES: c1ccc2c(c1)-c1ccccc1C21c2ccccc2-c2ccc(-n3c4ccc(-c5ccc6c(c5)c5c7ccccc7oc5n6-c5ccc6c(c5)C5(c7ccccc7-c7ccccc75)c5ccccc5-6)cc4c4c5ccccc5oc43)cc21 ; c1ccc2c(c1)ccc1cc(-n3c4ccc(-c5ccc6c(c5)c5c7ccccc7oc5n6-c5ccc6c(ccc7ccccc76)c5)cc4c4c5ccccc5oc43)ccc12]

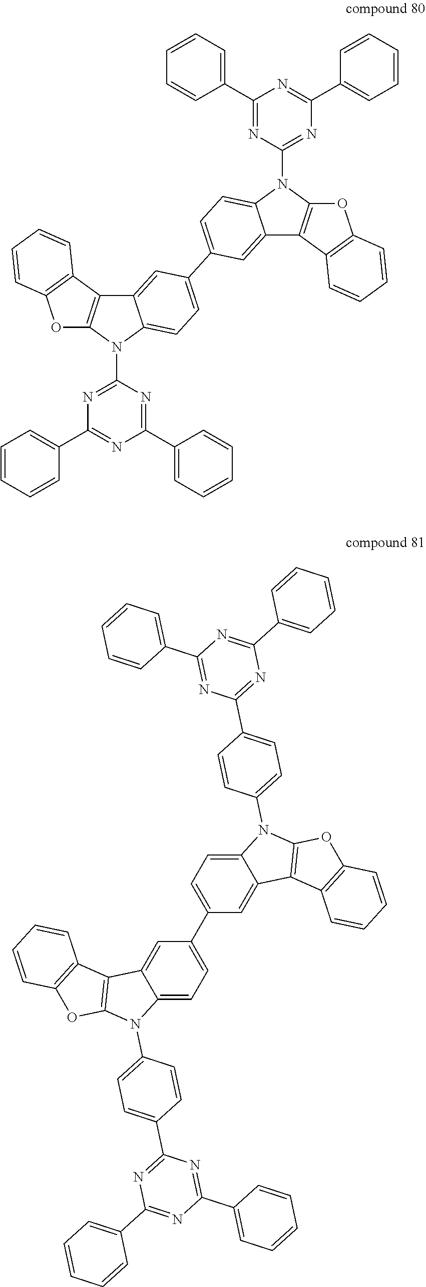 [SMILES: c1ccc(-c2nc(-c3ccccc3)nc(-n3c4ccc(-c5ccc6c(c5)c5c7ccccc7oc5n6-c5nc(-c6ccccc6)nc(-c6ccccc6)n5)cc4c4c5ccccc5oc43)n2)cc1 ; c1ccc(-c2nc(-c3ccccc3)nc(-c3ccc(-n4c5ccc(-c6ccc7c(c6)c6c8ccccc8oc6n7-c6ccc(-c7nc(-c8ccccc8)nc(-c8ccccc8)n7)cc6)cc5c5c6ccccc6oc54)cc3)n2)cc1]

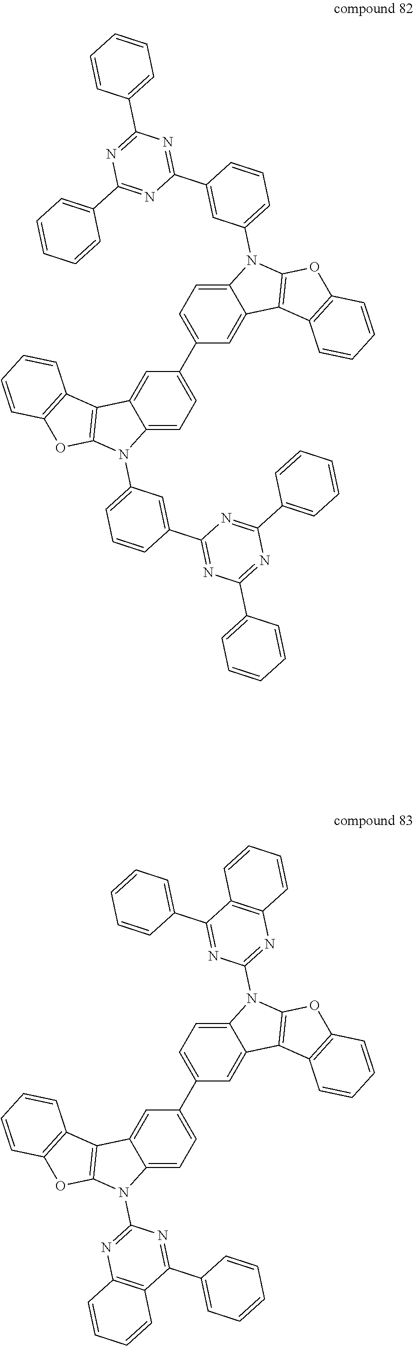 [SMILES: c1ccc(-c2nc(-c3ccccc3)nc(-c3cccc(-n4c5ccc(-c6ccc7c(c6)c6c8ccccc8oc6n7-c6cccc(-c7nc(-c8ccccc8)nc(-c8ccccc8)n7)c6)cc5c5c6ccccc6oc54)c3)n2)cc1 ; c1ccc(-c2nc(-n3c4ccc(-c5ccc6c(c5)c5c7ccccc7oc5n6-c5nc(-c6ccccc6)c6ccccc6n5)cc4c4c5ccccc5oc43)nc3ccccc23)cc1]

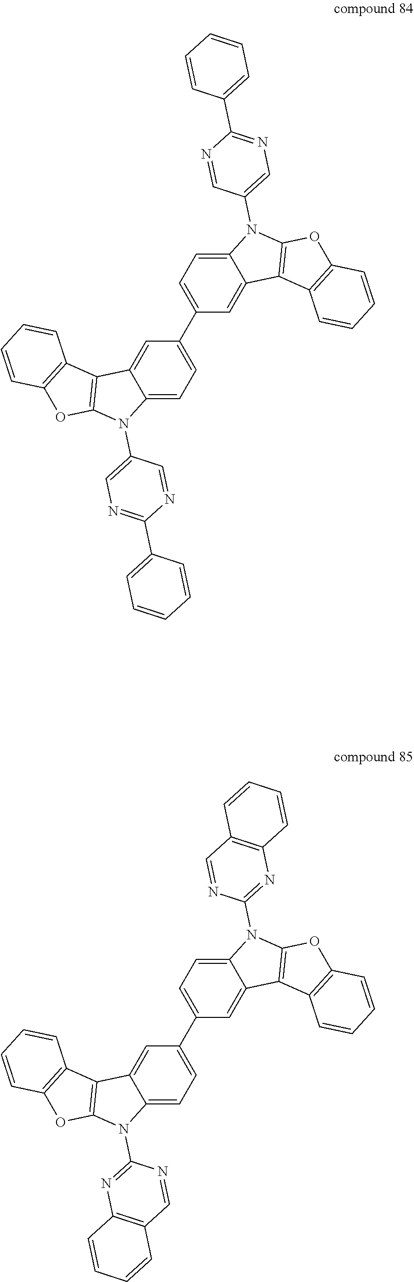 [SMILES: c1ccc(-c2ncc(-n3c4ccc(-c5ccc6c(c5)c5c7ccccc7oc5n6-c5cnc(-c6ccccc6)nc5)cc4c4c5ccccc5oc43)cn2)cc1 ; c1ccc2nc(-n3c4ccc(-c5ccc6c(c5)c5c7ccccc7oc5n6-c5ncc6ccccc6n5)cc4c4c5ccccc5oc43)ncc2c1]

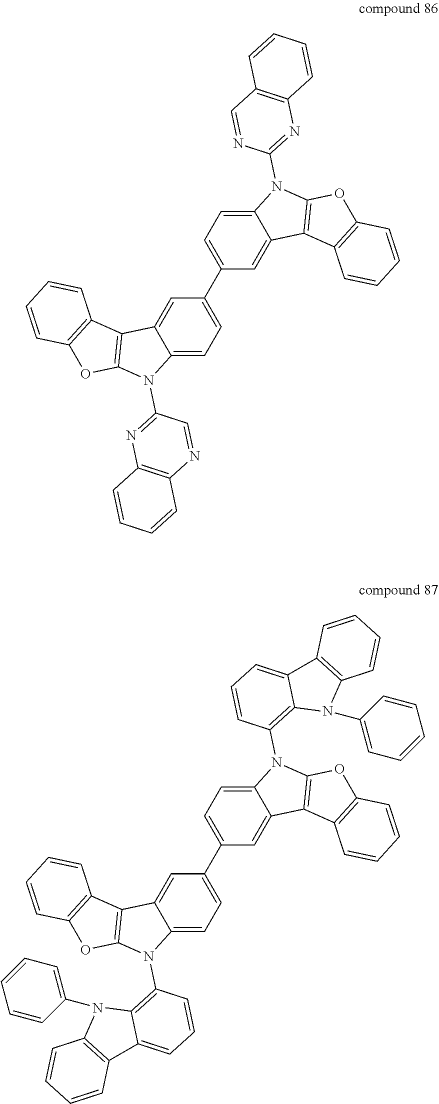 [SMILES: c1ccc2nc(-n3c4ccc(-c5ccc6c(c5)c5c7ccccc7oc5n6-c5cnc6ccccc6n5)cc4c4c5ccccc5oc43)ncc2c1 ; c1ccc(-n2c3ccccc3c3cccc(-n4c5ccc(-c6ccc7c(c6)c6c8ccccc8oc6n7-c6cccc7c8ccccc8n(-c8ccccc8)c67)cc5c5c6ccccc6oc54)c32)cc1]

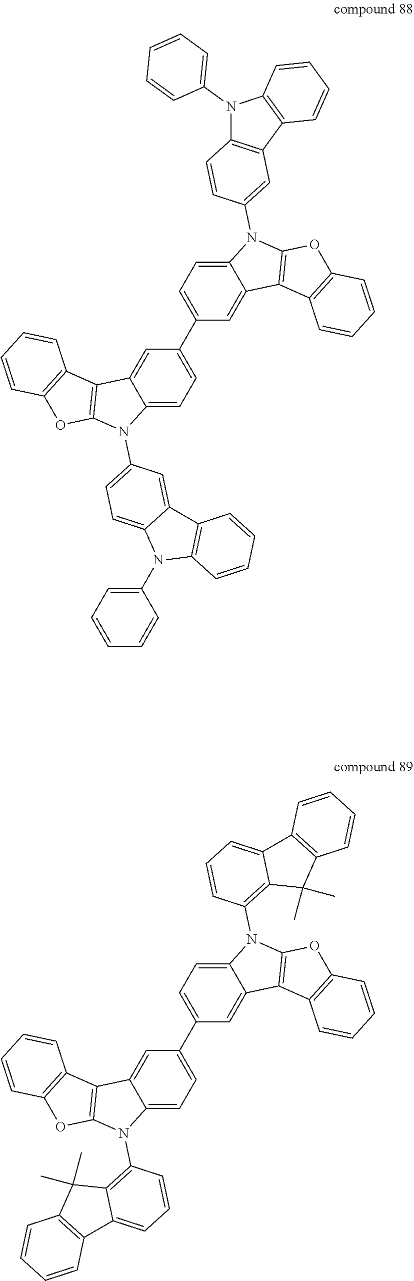 [SMILES: c1ccc(-n2c3ccccc3c3cc(-n4c5ccc(-c6ccc7c(c6)c6c8ccccc8oc6n7-c6ccc7c(c6)c6ccccc6n7-c6ccccc6)cc5c5c6ccccc6oc54)ccc32)cc1 ; CC1(C)c2ccccc2-c2cccc(-n3c4ccc(-c5ccc6c(c5)c5c7ccccc7oc5n6-c5cccc6c5C(C)(C)c5ccccc5-6)cc4c4c5ccccc5oc43)c21]

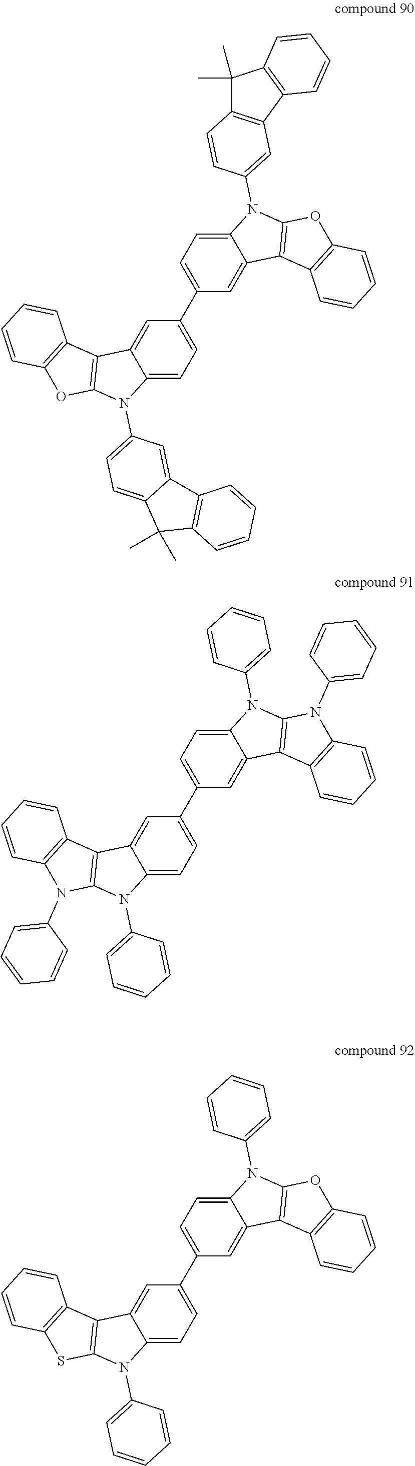 [SMILES: CC1(C)c2ccccc2-c2cc(-n3c4ccc(-c5ccc6c(c5)c5c7ccccc7oc5n6-c5ccc6c(c5)-c5ccccc5C6(C)C)cc4c4c5ccccc5oc43)ccc21 ; c1ccc(-n2c3ccccc3c3c4cc(-c5ccc6c(c5)c5c7ccccc7n(-c7ccccc7)c5n6-c5ccccc5)ccc4n(-c4ccccc4)c32)cc1 ; c1ccc(-n2c3ccc(-c4ccc5c(c4)c4c6ccccc6sc4n5-c4ccccc4)cc3c3c4ccccc4oc32)cc1]

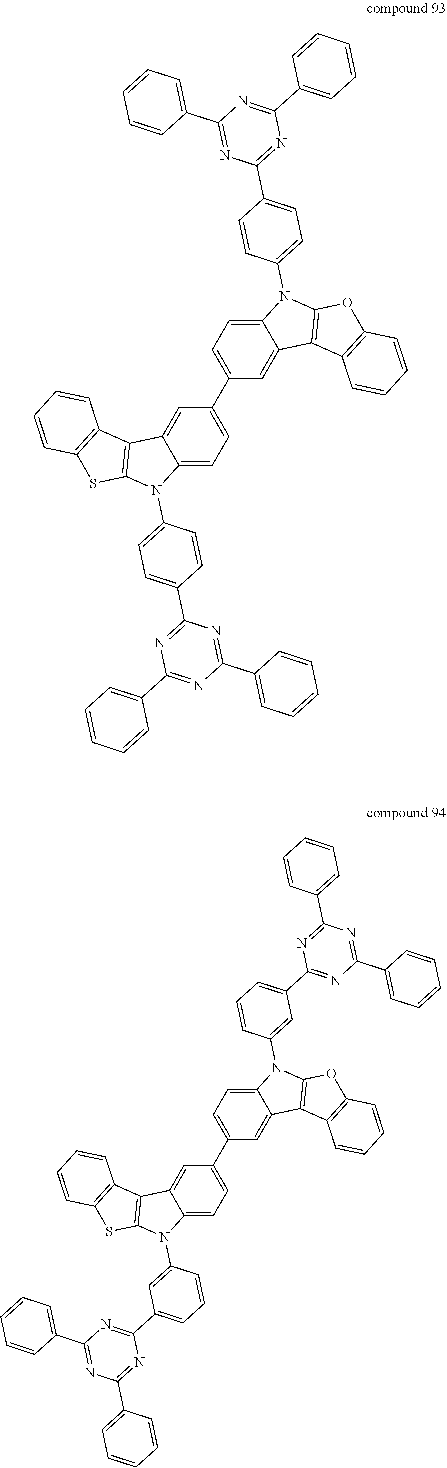 [SMILES: c1ccc(-c2nc(-c3ccccc3)nc(-c3ccc(-n4c5ccc(-c6ccc7c(c6)c6c8ccccc8sc6n7-c6ccc(-c7nc(-c8ccccc8)nc(-c8ccccc8)n7)cc6)cc5c5c6ccccc6oc54)cc3)n2)cc1 ; c1ccc(-c2nc(-c3ccccc3)nc(-c3cccc(-n4c5ccc(-c6ccc7c(c6)c6c8ccccc8sc6n7-c6cccc(-c7nc(-c8ccccc8)nc(-c8ccccc8)n7)c6)cc5c5c6ccccc6oc54)c3)n2)cc1]

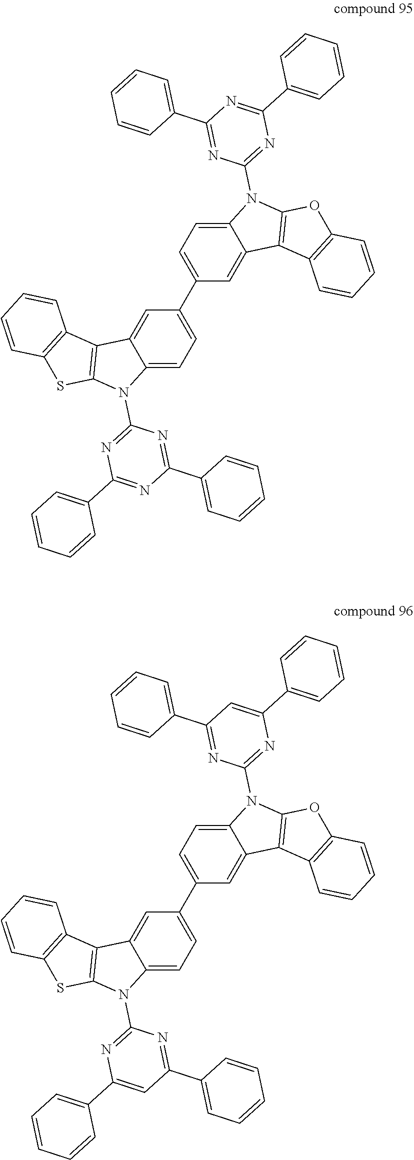 [SMILES: c1ccc(-c2nc(-c3ccccc3)nc(-n3c4ccc(-c5ccc6c(c5)c5c7ccccc7sc5n6-c5nc(-c6ccccc6)nc(-c6ccccc6)n5)cc4c4c5ccccc5oc43)n2)cc1 ; c1ccc(-c2cc(-c3ccccc3)nc(-n3c4ccc(-c5ccc6c(c5)c5c7ccccc7sc5n6-c5nc(-c6ccccc6)cc(-c6ccccc6)n5)cc4c4c5ccccc5oc43)n2)cc1]

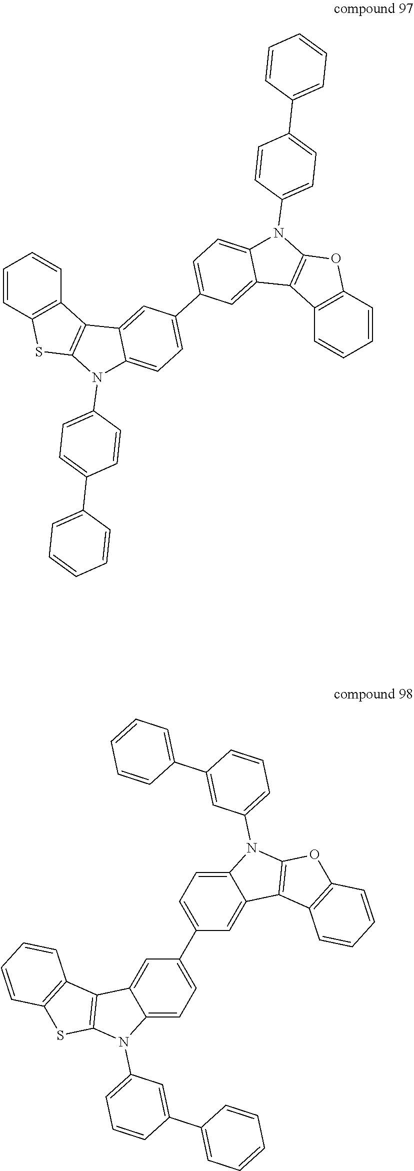 [SMILES: c1ccc(-c2ccc(-n3c4ccc(-c5ccc6c(c5)c5c7ccccc7sc5n6-c5ccc(-c6ccccc6)cc5)cc4c4c5ccccc5oc43)cc2)cc1 ; c1ccc(-c2cccc(-n3c4ccc(-c5ccc6c(c5)c5c7ccccc7sc5n6-c5cccc(-c6ccccc6)c5)cc4c4c5ccccc5oc43)c2)cc1]

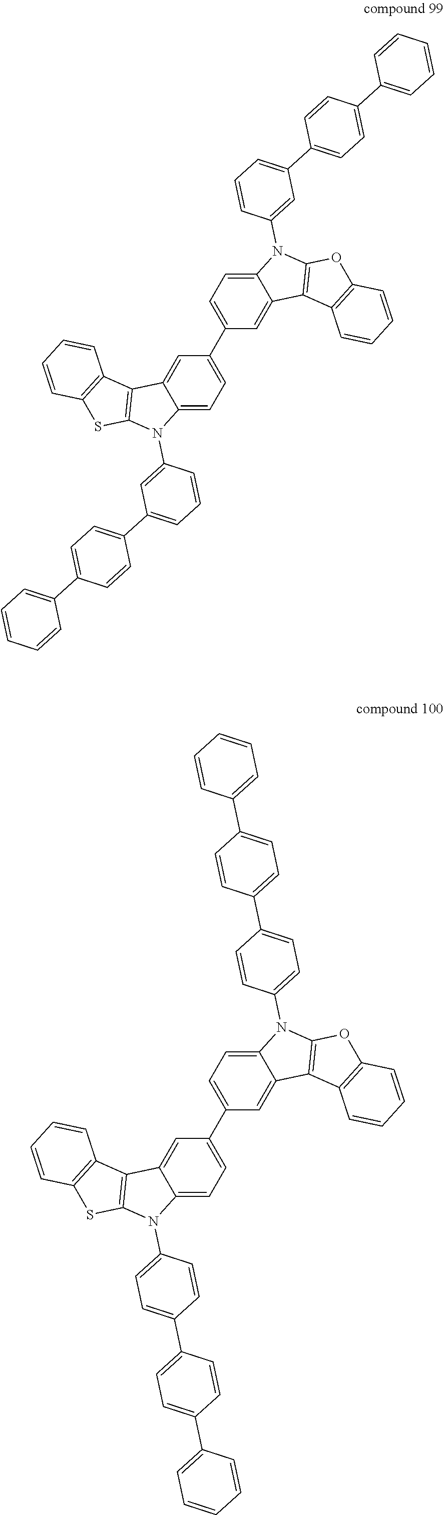 [SMILES: c1ccc(-c2ccc(-c3cccc(-n4c5ccc(-c6ccc7c(c6)c6c8ccccc8sc6n7-c6cccc(-c7ccc(-c8ccccc8)cc7)c6)cc5c5c6ccccc6oc54)c3)cc2)cc1 ; c1ccc(-c2ccc(-c3ccc(-n4c5ccc(-c6ccc7c(c6)c6c8ccccc8sc6n7-c6ccc(-c7ccc(-c8ccccc8)cc7)cc6)cc5c5c6ccccc6oc54)cc3)cc2)cc1]

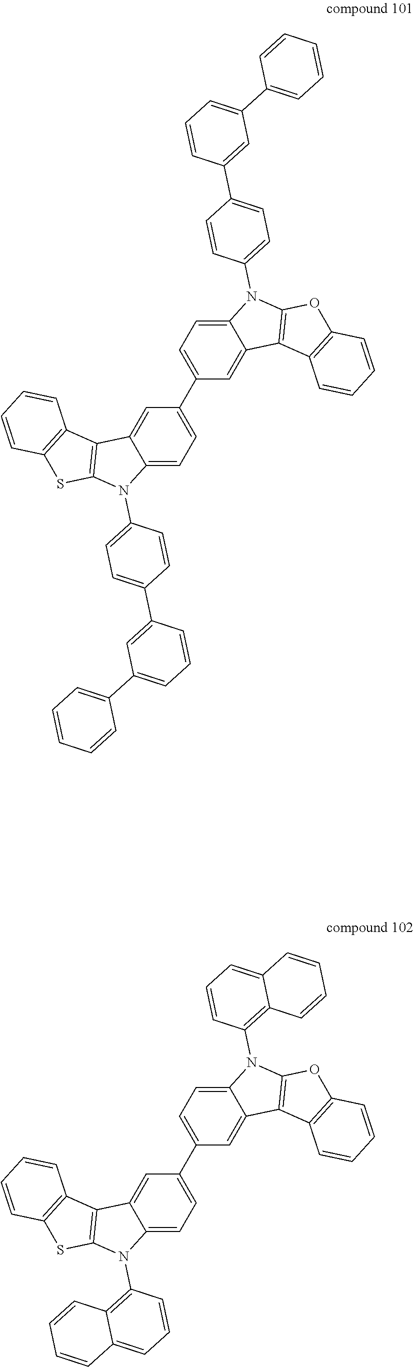 [SMILES: c1ccc(-c2cccc(-c3ccc(-n4c5ccc(-c6ccc7c(c6)c6c8ccccc8sc6n7-c6ccc(-c7cccc(-c8ccccc8)c7)cc6)cc5c5c6ccccc6oc54)cc3)c2)cc1 ; c1ccc2c(-n3c4ccc(-c5ccc6c(c5)c5c7ccccc7sc5n6-c5cccc6ccccc56)cc4c4c5ccccc5oc43)cccc2c1]

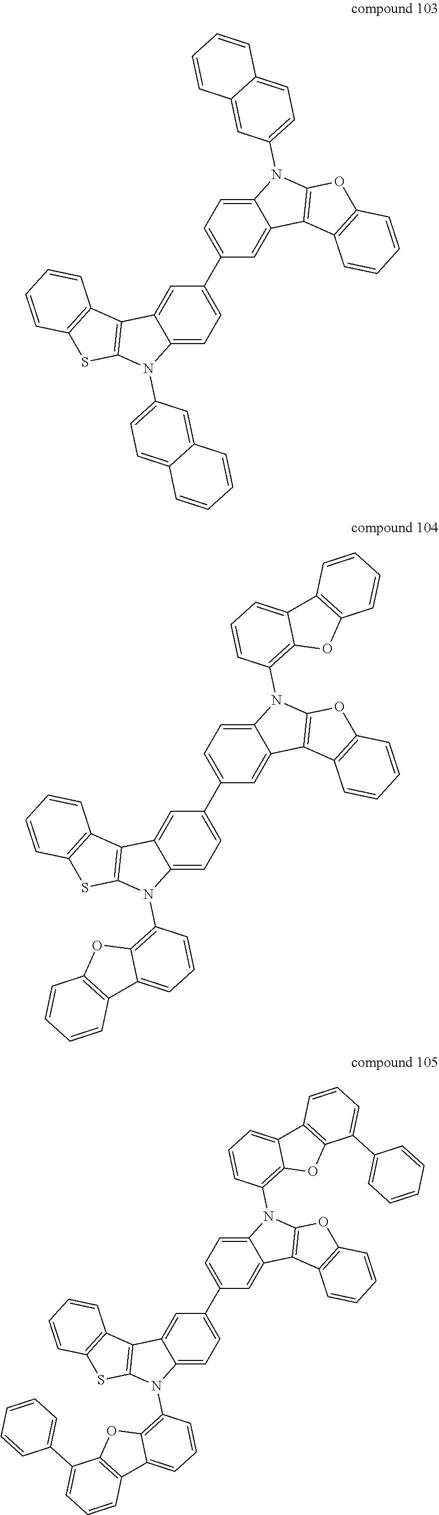 [SMILES: c1ccc2cc(-n3c4ccc(-c5ccc6c(c5)c5c7ccccc7sc5n6-c5ccc6ccccc6c5)cc4c4c5ccccc5oc43)ccc2c1 ; c1ccc2c(c1)oc1c(-n3c4ccc(-c5ccc6c(c5)c5c7ccccc7sc5n6-c5cccc6c5oc5ccccc56)cc4c4c5ccccc5oc43)cccc12 ; c1ccc(-c2cccc3c2oc2c(-n4c5ccc(-c6ccc7c(c6)c6c8ccccc8sc6n7-c6cccc7c6oc6c(-c8ccccc8)cccc67)cc5c5c6ccccc6oc54)cccc23)cc1]

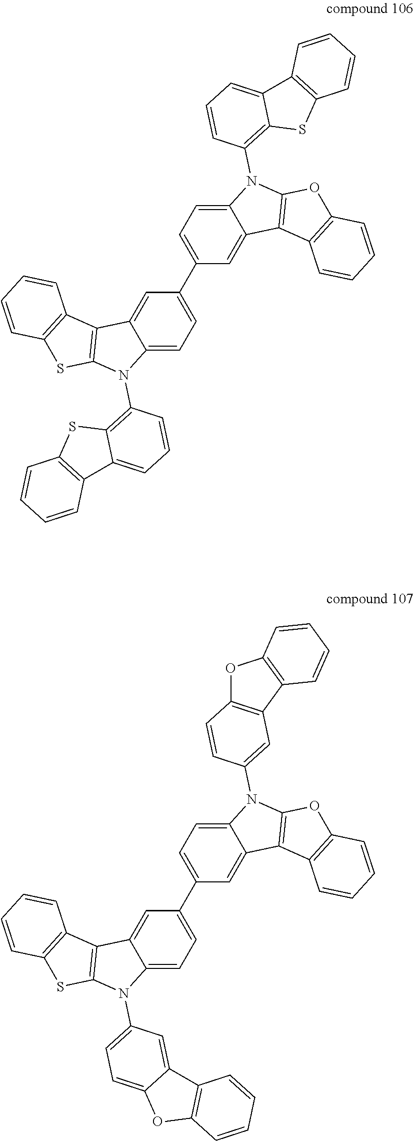 [SMILES: c1ccc2c(c1)oc1c2c2cc(-c3ccc4c(c3)c3c5ccccc5sc3n4-c3cccc4c3sc3ccccc34)ccc2n1-c1cccc2c1sc1ccccc12 ; c1ccc2c(c1)oc1ccc(-n3c4ccc(-c5ccc6c(c5)c5c7ccccc7sc5n6-c5ccc6oc7ccccc7c6c5)cc4c4c5ccccc5oc43)cc12]

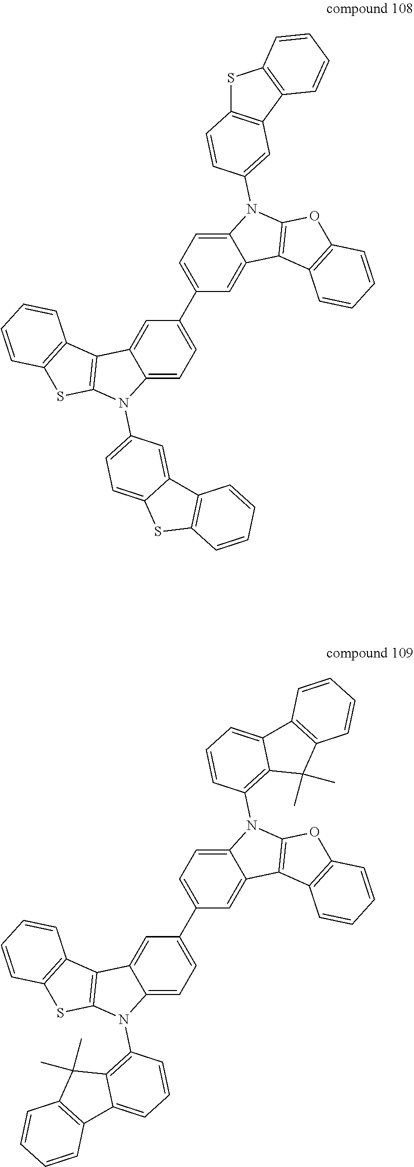 [SMILES: c1ccc2c(c1)oc1c2c2cc(-c3ccc4c(c3)c3c5ccccc5sc3n4-c3ccc4sc5ccccc5c4c3)ccc2n1-c1ccc2sc3ccccc3c2c1 ; CC1(C)c2ccccc2-c2cccc(-n3c4ccc(-c5ccc6c(c5)c5c7ccccc7sc5n6-c5cccc6c5C(C)(C)c5ccccc5-6)cc4c4c5ccccc5oc43)c21]

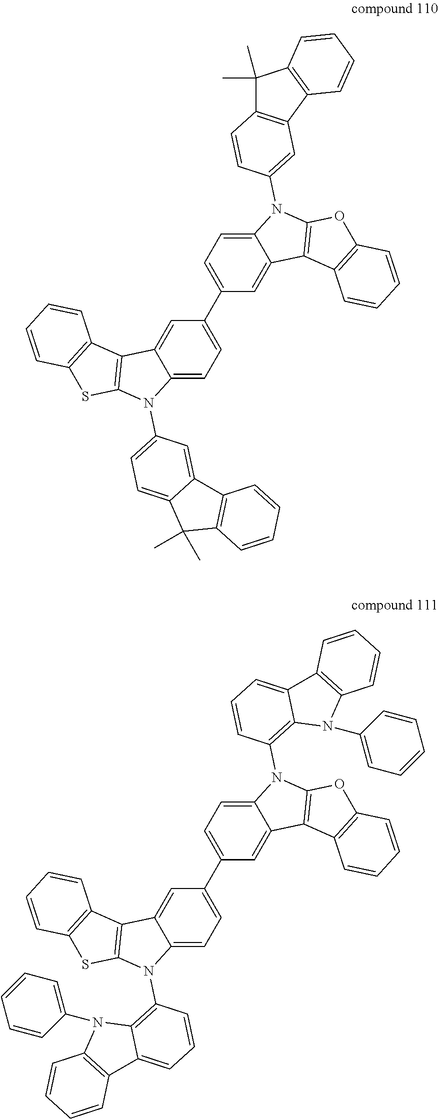 [SMILES: CC1(C)c2ccccc2-c2cc(-n3c4ccc(-c5ccc6c(c5)c5c7ccccc7sc5n6-c5ccc6c(c5)-c5ccccc5C6(C)C)cc4c4c5ccccc5oc43)ccc21 ; c1ccc(-n2c3ccccc3c3cccc(-n4c5ccc(-c6ccc7c(c6)c6c8ccccc8sc6n7-c6cccc7c8ccccc8n(-c8ccccc8)c67)cc5c5c6ccccc6oc54)c32)cc1]

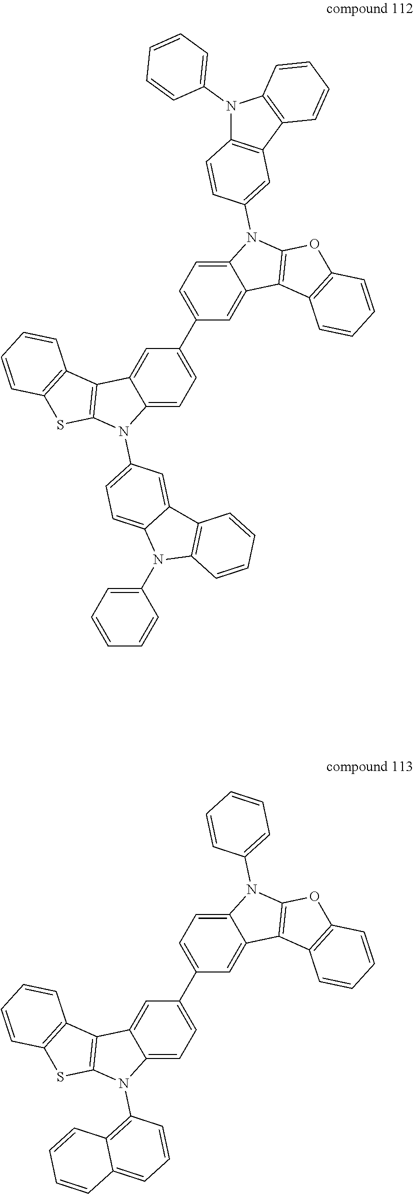 [SMILES: c1ccc(-n2c3ccccc3c3cc(-n4c5ccc(-c6ccc7c(c6)c6c8ccccc8sc6n7-c6ccc7c(c6)c6ccccc6n7-c6ccccc6)cc5c5c6ccccc6oc54)ccc32)cc1 ; c1ccc(-n2c3ccc(-c4ccc5c(c4)c4c6ccccc6sc4n5-c4cccc5ccccc45)cc3c3c4ccccc4oc32)cc1]

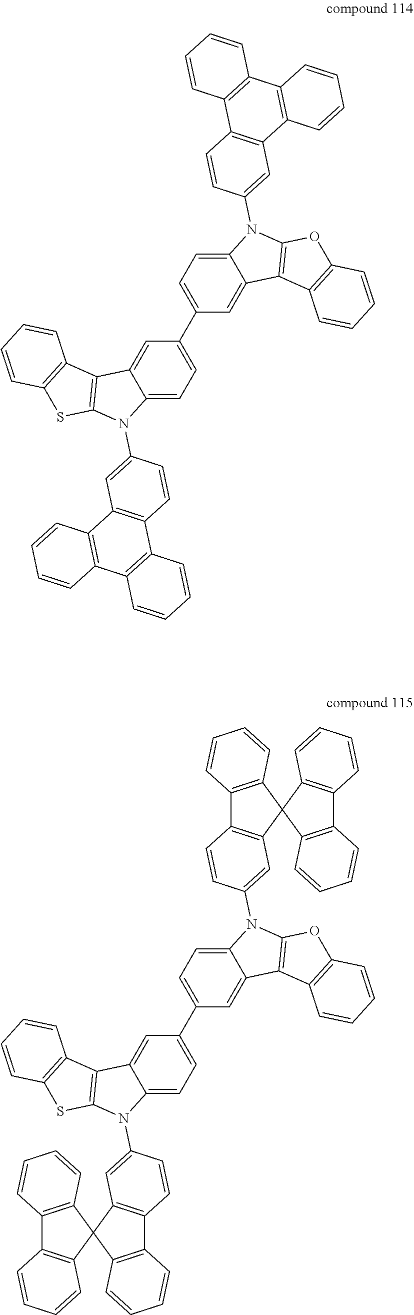 [SMILES: c1ccc2c(c1)oc1c2c2cc(-c3ccc4c(c3)c3c5ccccc5sc3n4-c3ccc4c5ccccc5c5ccccc5c4c3)ccc2n1-c1ccc2c3ccccc3c3ccccc3c2c1 ; c1ccc2c(c1)-c1ccccc1C21c2ccccc2-c2ccc(-n3c4ccc(-c5ccc6c(c5)c5c7ccccc7sc5n6-c5ccc6c(c5)C5(c7ccccc7-c7ccccc75)c5ccccc5-6)cc4c4c5ccccc5oc43)cc21]

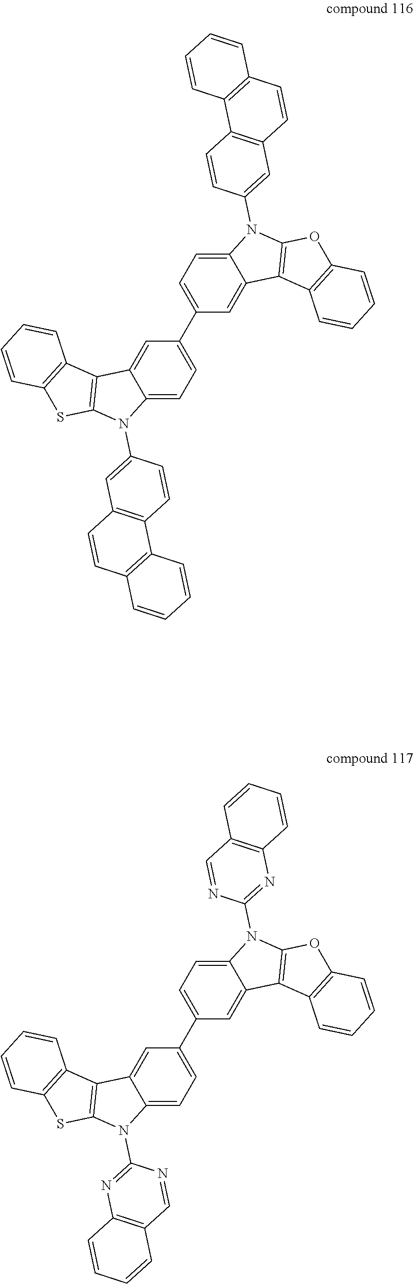 [SMILES: c1ccc2c(c1)ccc1cc(-n3c4ccc(-c5ccc6c(c5)c5c7ccccc7sc5n6-c5ccc6c(ccc7ccccc76)c5)cc4c4c5ccccc5oc43)ccc12 ; c1ccc2nc(-n3c4ccc(-c5ccc6c(c5)c5c7ccccc7sc5n6-c5ncc6ccccc6n5)cc4c4c5ccccc5oc43)ncc2c1]

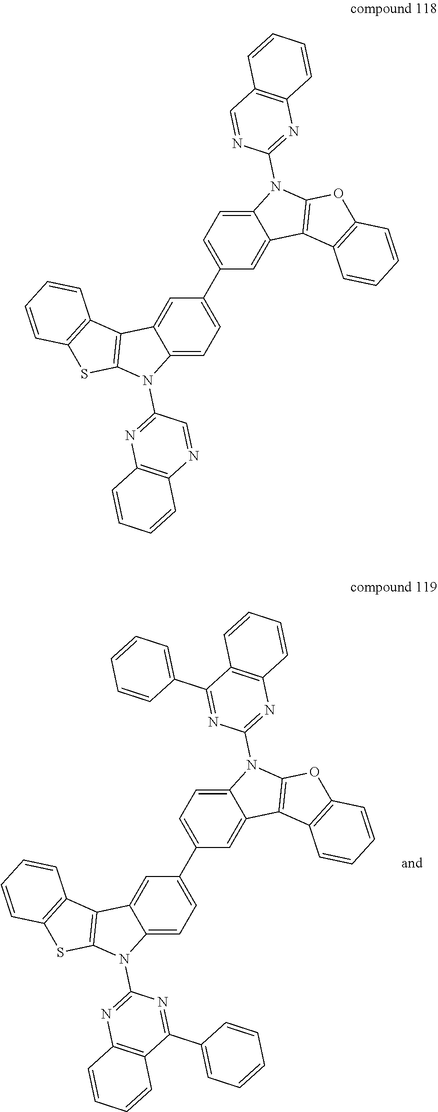 [SMILES: c1ccc2nc(-n3c4ccc(-c5ccc6c(c5)c5c7ccccc7sc5n6-c5cnc6ccccc6n5)cc4c4c5ccccc5oc43)ncc2c1 ; c1ccc(-c2nc(-n3c4ccc(-c5ccc6c(c5)c5c7ccccc7sc5n6-c5nc(-c6ccccc6)c6ccccc6n5)cc4c4c5ccccc5oc43)nc3ccccc23)cc1]

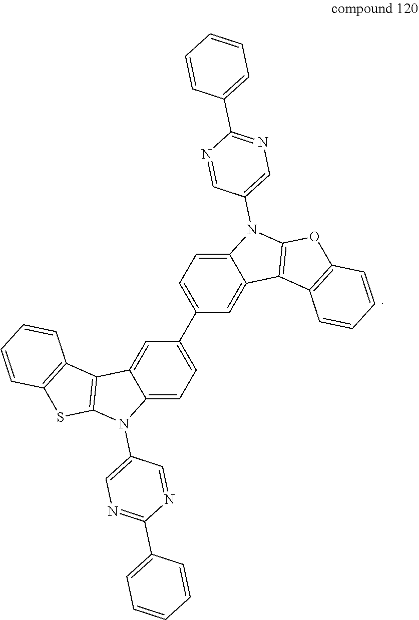 [SMILES: c1ccc(-c2ncc(-n3c4ccc(-c5ccc6c(c5)c5c7ccccc7sc5n6-c5cnc(-c6ccccc6)nc5)cc4c4c5ccccc5oc43)cn2)cc1]

An organic electroluminescence device comprising a pair of electrodes having an anode, a cathode and one or more organic layers formed between the anode and the cathode. At least one of the organic layers comprises the organic compound of formula (1).

The organic layers may comprise an emissive layer having a host. In one embodiment, the organic compound of formula (1) is comprised as the host.

The organic layers may comprise a hole transporting layer. In one embodiment, the organic compound of formula (1) is comprised as the hole transporting layer.

The organic layers may comprise an electron transporting layer. In one embodiment, the organic compound of formula (1) is comprised as the electron transporting layer.

The organic layers may comprise an electron transporting layer. In one embodiment, the organic compound of formula (1) is comprised as the electron transporting layer.

The organic layers may comprise an electron blocking layer. In one embodiment, the organic compound of formula (1) is comprised as the electron blocking layer.

The organic layers may comprise a hole blocking layer. In one embodiment, the organic compound of formula (1) is comprised as the hole blocking layer.

In one embodiment, the organic electroluminescence device is a lighting panel.

In one embodiment, the organic electroluminescence device is a backlight panel.

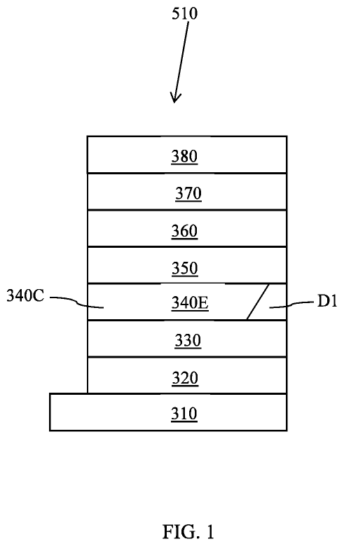

In one embodiment, a first organic EL device comprising the organic compound of formula (1) is disclosed. FIG. 1 is a cross-sectional view of the first organic EL device. Referring to FIG. 1, the first organic EL device 510 may comprise the organic compound of formula (1) as a host 340C of an emissive layer 340E.

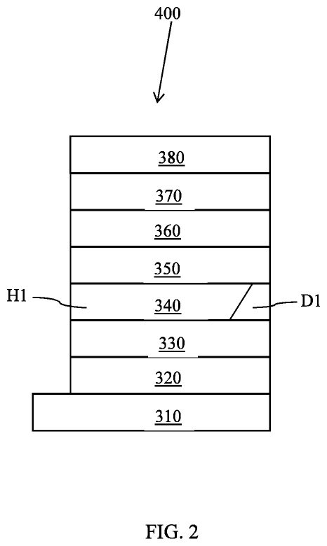

FIG. 2 is a cross-sectional view of an organic EL device without the organic compound of formula (1) (without 340C of FIG. 1). Referring to FIG. 2, the organic EL device 400 may have a driving voltage of about 5.1 V, a current efficiency of about 18 cd/A, or a half-life of about 350 hours.

Referring to FIG. 1, by comprising the organic compound of formula (1) as the host 340C, the first organic EL device 510 may have a driving voltage lower than that of the organic EL device 400 (FIG. 2). Moreover, by comprising the organic compound of formula (1) as the host 340C, the first organic EL device 510 of FIG. 1 may have a current efficiency higher than that of the organic EL device 400 (FIG. 2). Furthermore, by comprising the organic compound of formula (1) as the host 340C, the first organic EL device 510 of FIG. 1 may have a half-life longer than that of the organic EL device 400 (FIG. 2).

As the host 340C of the first organic EL device 510 of FIG. 1, the organic compound of formula (1) may lower the driving voltage to be about 3.0 V to about 4.7 V. Moreover, the organic compound of formula (1) may increase the current efficiency to be 30 cd/A to about 45 cd/A. Furthermore, the organic compound of formula (1) may increase the half-life to be about 428 hours to about 980 hours.

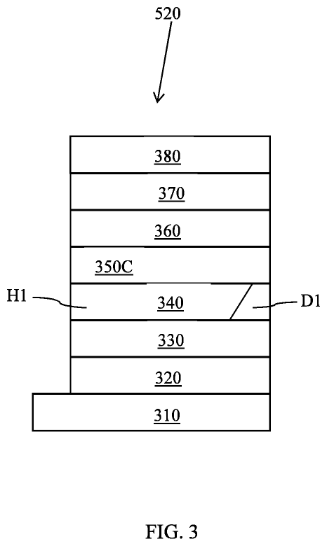

In a third embodiment of the present invention, a second organic EL device using the organic compound of formula (1) is disclosed. FIG. 3 is a cross-sectional view of the second organic EL device. Referring to FIG. 3, the second organic EL device 520 may comprise the organic compound of formula (1) as a hole blocking layer 350C.

FIG. 2 is a cross-sectional view of an organic EL device without the organic compound of formula (1) (without 350C of FIG. 3). Referring to FIG. 2, the organic EL device 400 may have a driving voltage of about 5.1 V, a current efficiency of about 18 cd/A, or a half-life of about 350 hours.

Referring to FIG. 3, by comprising the organic compound of formula (1) as the hole blocking layer 350C, the second organic EL device 520 may have a driving voltage lower than that of the organic EL device 400 (FIG. 2). Moreover, by comprising the organic compound of formula (1) as the hole blocking layer 350C, the second organic EL device 520 of FIG. 3 may have a current efficiency higher than that of the organic EL device 400 (FIG. 2). Furthermore, by comprising the organic compound of formula (1) as the hole blocking layer 350C, the second organic EL device 520 of FIG. 3 may have a half-life longer than that of the organic EL device 400 (FIG. 2).

Referring to FIG. 3, as the hole blocking layer 350C of the second organic EL device 520, the organic compound of formula (1) may lower the driving voltage to be about 4.2 V to about 4.8 V. Moreover, the organic compound of formula (1) may increase the current efficiency to be about 20 cd/A to about 27 cd/A. Furthermore, the organic compound of formula (1) may increase the half-life to be about 370 hours to about 510 hours.

Referring to FIG. 1, the first organic EL device 510 may comprise an anode 310, a cathode 380 and one or more organic layers 320, 330, 340E, 350, 360, 370 formed between the anode 310 and the cathode 380. From the bottom to the top, the one or more organic layers may comprise a hole injection layer 320, a hole transport layer 330, an emissive layer 340E, a hole blocking layer 350, an electron transport layer 360 and an electron injection layer 370.

The emissive layer 340E may comprise a 15% dopant D1 and the organic compound of formula (1) 340C doped with the dopant D1. The dopant D1 may be a green guest material for tuning the wavelength at which the emissive layer 340E emits light, so that the color of emitted light may be green. The color may be measured using CIE coordinates, which are well known to the art. The organic compound of formula (1) may be a host 340C of the emissive layer 340E.

FIG. 2 is a cross-sectional view of an organic EL device without the organic compound of formula (1). Referring to FIG. 2, the organic EL device 400 may comprise an anode 310, a cathode 380 and one or more organic layers 320, 330, 340, 350, 360, 370 formed between the anode 310 and the cathode 380. From the bottom to the top, the one or more organic layers may comprise a hole injection layer 320, a hole transport layer 330, an emissive layer 340, a hole blocking layer 350, an electron transport layer 360 and an electron injection layer 370. The emissive layer 340 may comprise a 15% dopant D1 and an organic compound H1 doped with the dopant D1. The dopant D1 may be a green guest material. The organic compound H1 is a host of the emissive layer 340.

To those organic EL devices of FIG. 1 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 1 and FIG. 2 may be summarized in Table 1 below. The half-life is defined as the time that the initial luminance of 1000 cd/m² has dropped to half.

TABLE 1

| | | Driving | Current | | |
|---|---|---|---|---|---|
| Host | | Voltage | Efficiency | | Half-life |
| (H1 or 340C) | Dopant | (V) | (cd/A) | CIE (y) | (hours) |
| H1 | D1 | 5.1 | 18 | 0.53 | 350 |
| Comp. 4 | D1 | 3.0 | 45 | 0.52 | 980 |
| Comp. 7 | D1 | 4.6 | 36 | 0.56 | 440 |
| Comp. 9 | D1 | 3.7 | 37 | 0.54 | 520 |
| Comp. 21 | D1 | 4.4 | 30 | 0.53 | 580 |
| Comp. 23 | D1 | 4.3 | 33 | 0.55 | 530 |
| Comp. 26 | D1 | 4.5 | 27 | 0.54 | 430 |
| Comp. 28 | D1 | 3.7 | 36 | 0.54 | 680 |
| Comp. 38 | D1 | 4.7 | 44 | 0.54 | 428 |
| Comp. 39 | D1 | 3.8 | 36 | 0.53 | 690 |
| Comp. 41 | D1 | 3.7 | 37 | 0.55 | 700 |
| Comp. 44 | D1 | 3.0 | 43 | 0.53 | 920 |
| Comp. 45 | D1 | 3.1 | 42 | 0.52 | 810 |
| Comp. 48 | D1 | 3.0 | 40 | 0.53 | 770 |
| Comp. 77 | D1 | 4.5 | 36 | 0.54 | 510 |
| Comp. 80 | D1 | 3.0 | 45 | 0.52 | 888 |
| Comp. 86 | D1 | 3.2 | 43 | 0.53 | 860 |
| Comp. 91 | D1 | 3.3 | 40 | 0.55 | 800 |
| Comp. 92 | D1 | 3.3 | 42 | 0.54 | 810 |
| Comp. 95 | D1 | 3.3 | 43 | 0.53 | 862 |
| Comp. 98 | D1 | 3.3 | 36 | 0.55 | 563 |
| Comp. 115 | D1 | 4.3 | 38 | 0.54 | 511 |
| Comp. 116 | D1 | 3.2 | 38 | 0.53 | 588 |
| Comp. 120 | D1 | 3.3 | 41 | 0.53 | 830 |

(The “Comp.” is short for “Compound”)

According to Table 1, in the first organic EL device 510, the organic compound of formula (1) comprised as a host 340C of FIG. 1 exhibits performance better than a prior art organic EL material (H1).

A method of producing the first organic EL device 510 of FIG. 1 and the organic EL device 400 of FIG. 2 is described. ITO-coated glasses with 9-12 ohm/square in resistance and 120-160 nm in thickness are provided (hereinafter ITO substrate) and cleaned in a number of cleaning steps in an ultrasonic bath (e.g. detergent, deionized water).

Before vapor deposition of the organic layers, cleaned ITO substrates may be further treated by UV and ozone. All pre-treatment processes for ITO substrate are under clean room (class 100), so that an anode 310 may be formed.

One or more organic layers 320, 330, 340 (FIG. 2), 340E (FIG. 1), 350, 360, 370 are applied onto the anode 310 in order by vapor deposition in a high-vacuum unit (10⁻⁷ Torr), such as resistively heated quartz boats. The thickness of the respective layer and the vapor deposition rate (0.1-0.3 nm/sec) are precisely monitored or set with the aid of a quartz-crystal monitor. It is also possible, as described above, each of the organic layers may comprise more than one organic compound. For example, an emissive layer 340E or 340 may be formed of a dopant and a host doped with the dopant. An emissive layer 340E or 340 may also be formed of a co-host and a host co-deposited with the co-host. This may be successfully achieved by co-vaporization from two or more sources. Accordingly, the compounds for the organic layers of the present invention are thermally stable.

Dipyrazino[2,3-f:2,3-] quinoxaline-2,3,6,7,10,11-hexacarbonitrile (HAT-CN) may be applied to form a hole injection layer (HIL) 320 having a thickness of about 20 nm in the organic EL device 510 or 400. N,N-Bis(naphthalene-1-yl)-N,N-bis(phenyl)-benzidine (NPB) may be applied to form a hole transporting layer (HTL) 330 having a thickness of about 110 nm.

Referring to FIG. 1 and FIG. 2, in the organic EL device 510 (FIG. 1) or 400 (FIG. 2), an emissive layer (EML) 340E or 340 may be formed to have a thickness of about 30 nm. Referring to FIG. 2, in the organic EL device 400, 12-(4,6-diphenyl-1,3,5-triazin-2-yl) -10,10-dimethyl-10,12-dihydrophenanthro[9′,10′1:5,6]indeno[2,1-b]carbazole (i.e., H1 of paragraph [0002]) may be applied to form a host H1 of an emissive layer 340 of FIG. 2. The emissive layer 340 may further comprise bis(2-phenylpyridinato)(2,4-diphenylpyridinato)-iridium(III) as a dopant D1, also a green guest of the emissive layer 340. On the emissive layer 340 having a thickness of about 30 nm, a compound HB1 may be a hole blocking material (HBM) to form a hole blocking layer (HBL) 350 having a thickness of about 10 nm.

2-(naphthalen-1-yl)-9-(4-(1-(4-(10-(naphthalene-2-yl)anthracen-9-yl)-phenyl) -1H-benzo[d]imidazol-2-yl)phenyl)-1,10-phenanthroline (ET1) may be applied as an electron transporting material to co-deposit with 8-hydroxyquinolato-lithium (LiQ) at a ratio of 1:1, thereby forming an electron transporting layer 360 of the organic EL device 510 or 400. The electron transporting layer (ETL) 360 may have a thickness of about 35 nm.

Table 2 shows the layer thickness and materials of the organic EL device 510 (FIG. 1) or 400 (FIG. 2).

TABLE 2

| Ref. No. in | | | Thickness |
|---|---|---|---|
| FIG. 1 or FIG. 2 | Layer | Material | (nm) |
| 380 | Cathode | Al | 160 |
| 370 | EIL | LiQ | 1 |
| 360 | ETL | LiQ (50%):ET1 (50%) | 35 |
| 350 | HBL | HB1 | 10 |
| 340E (FIG. 1) | EML | 340C (85%):D1 (15%) | 30 |
| or | | or | |
| 340 (FIG. 2) | | H1 (85%):D1 (15%) | |
| 330 | HTL | NPB | 110 |
| 320 | HIL | HAT-CN | 20 |
| 310 | Anode | ITO substrate | 120~160 |

The organic compounds ET1, HB1, D1, NPB and HAT-CN for producing the organic EL device 400 or 510 in this invention may have the formulas as follows:

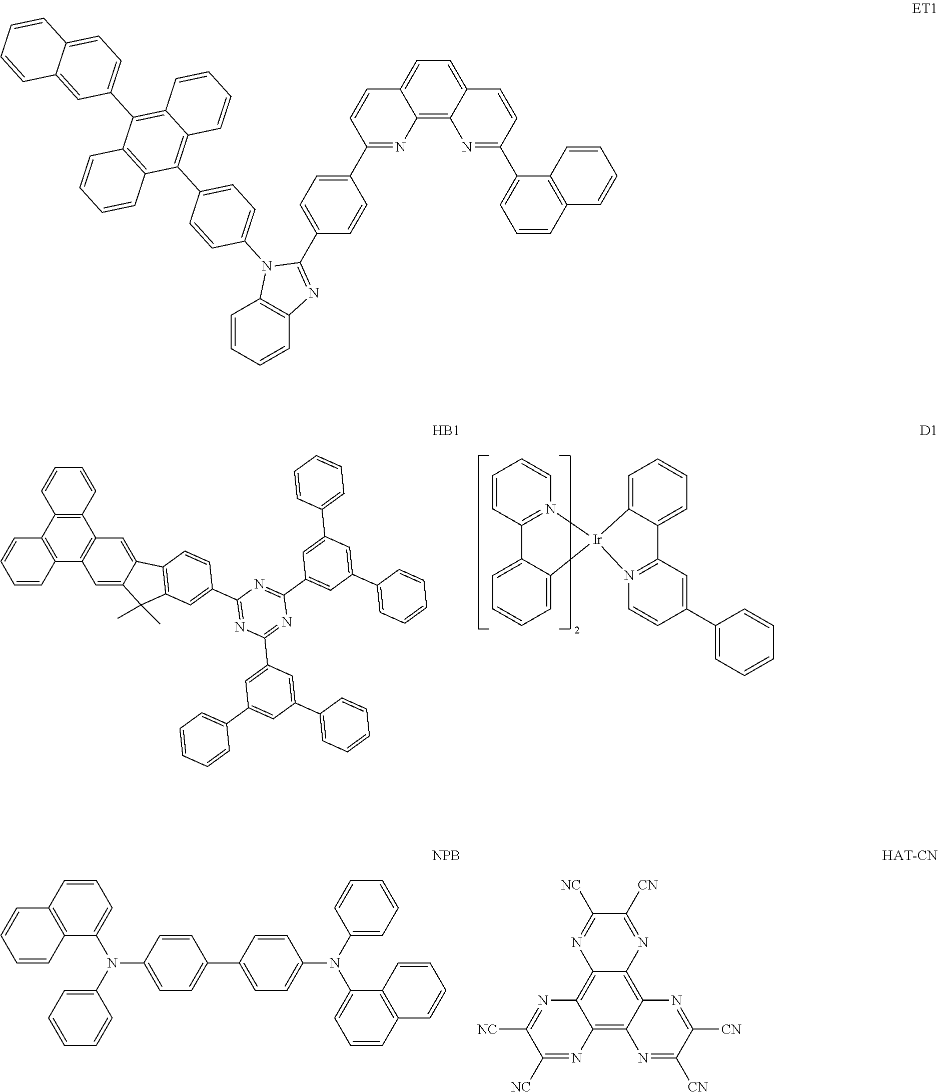

Referring to FIG. 1 and FIG. 2, the organic EL device 510 or 400 may further comprise a low work function metal, such as Al, Mg, Ca, Li or K, as a cathode 380 by thermal evaporation. The cathode 380 having a thickness of about 160 nm may help electrons injecting the electron transporting layer 360 from cathode 380. Between the cathode 380 (e.g., A1 in Table 2) and the electron transporting layer 360, a thin electron injecting layer (EIL) 370 of LiQ is introduced. The electron injecting layer (EIL) 370 has a thickness of about 1 nm is to reduce the electron injection barrier and to improve the performance of the organic EL device 510 or 400. The material of the electron injecting layer 370 may alternatively be metal halide or metal oxide with low work function, such as LiF, MgO, or Li₂O.

In any above-mentioned compounds used in each layer of an organic EL device, the hydrogen atoms can be partially or fully deuterated. Thus, any specifically listed substituent, such as, without limitation, methyl, phenyl, pyridyl, etc. may be undeuterated, partially deuterated, and fully deuterated versions thereof. Similarly, classes of substituents such as, without limitation, alkyl, aryl, cycloalkyl, heteroaryl, etc. also may be undeuterated, partially deuterated, and fully deuterated versions thereof.

In one embodiment, a second organic EL device using the organic compound of formula (1) is disclosed. The method of producing the second organic EL device 520 of FIG. 3 is substantially the same as the method of producing the organic EL device 400 of FIG. 2. The difference is that the hole blocking layer (HBL) 350C of FIG. 3 is made by using the organic compound of formula (1), rather than HB1.

Table 3 shows the layer thickness and materials of the organic EL device 520 (FIG. 3) or 400 (FIG. 2).

TABLE 3

| Ref. No. in | | | Thickness |
|---|---|---|---|
| FIG.1 or FIG. 2 | Layer | Material | (nm) |
| 380 | Cathode | Al | 160 |
| 370 | EIL | LiQ | 1 |
| 360 | ETL | LiQ:ET1 (50%) | 35 |
| 350C | HBL | 350C | 10 |
| or | | or | |
| 350 | | HB1 | |
| 340 | EML | H1:D1 (15%) | 30 |
| 330 | HTL | NPB | 110 |
| 320 | HIL | HAT-CN | 20 |
| 310 | Anode | ITO substrate | 120~160 |

To those organic EL devices of FIG. 3 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 3 and FIG. 2 may be summarized in Table 4 below. The half-life of the fluorescent green-emitting organic EL device 520 or 400 is defined as the time that the initial luminance of 1000 cd/m² has dropped to half.

TABLE 4

| | ETM | Driving | Current | | |
|---|---|---|---|---|---|
| Material for | for | Voltage | Efficiency | | Half-life |
| HBL 350 or 350C | ETL 360 | (V) | (cd/A) | CIE(y) | (hours) |
| HB1 | ET1 | 5.1 | 18 | 0.53 | 350 |
| Comp. 4 | ET1 | 4.4 | 23 | 0.52 | 420 |
| Comp. 7 | ET1 | 4.5 | 24 | 0.55 | 410 |
| Comp. 9 | ET1 | 4.2 | 25 | 0.53 | 440 |
| Comp. 21 | ET1 | 4.3 | 25 | 0.54 | 470 |
| Comp. 39 | ET1 | 4.3 | 25 | 0.55 | 440 |
| Comp. 41 | ET1 | 4.3 | 27 | 0.52 | 500 |
| Comp. 44 | ET1 | 4.3 | 23 | 0.54 | 480 |
| Comp. 45 | ET1 | 4.3 | 27 | 0.52 | 510 |
| Comp. 77 | ET1 | 4.5 | 24 | 0.53 | 470 |
| Comp. 80 | ET1 | 4.3 | 26 | 0.53 | 503 |
| Comp. 86 | ET1 | 4.3 | 26 | 0.53 | 490 |
| Comp. 91 | ET1 | 4.8 | 20 | 0.52 | 370 |
| Comp. 98 | ET1 | 4.5 | 23 | 0.54 | 380 |

According to Table 4, in the second organic EL device 520, the organic compound of formula (1) comprised as a hole blocking layer 350C of FIG. 3 exhibits performance better than a prior art hole blocking material (HB1 as a HBL 350 of FIG. 2).

Referring to FIG. 1 or FIG. 3, the organic EL device 510 or 520 of the present invention may alternatively be a lighting panel or a backlight panel.

Detailed preparation of the organic compounds of the present invention will be clarified by exemplary embodiments below, but the present invention is not limited thereto. EXAMPLES 1 to 23 show the preparation of the organic compounds of the present invention.

### EXAMPLE 1

Synthesis of Intermediate A

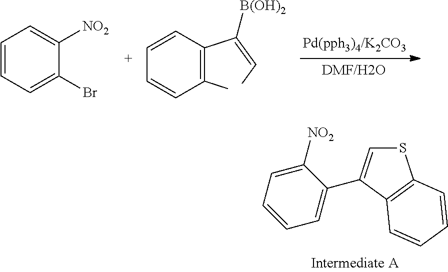 [SMILES: O=[N+]([O-])c1ccccc1Br ; OB(O)C1=CCc2ccccc21 ; O=[N+]([O-])c1ccccc1-c1csc2ccccc12]

A mixture of 20 g (99 mmole) of 1-Bromo-2-nitrobenzene, 19.3 g (108.9 mmole) of benzo[b]thiophen-3-ylboronic acid, 2.2 g (1.98 mmole) of Pd(pph₃)₄, 27.4 g (198.2 mmole) of K₂CO₃, 300 ml of DMF, and 80 ml of H₂O was placed under nitrogen, and then heated at 80° C. while stirring for 5 h. After the reaction was finished, the mixture was allowed to cool to room temperature. The solution was extracted with 100 ml of ethyl acetate (3 times) and 300 ml of water. The organic layer was dried with anhydrous magnesium sulfate and the solvent was evaporated under reduced pressure. The residue was purified by column chromatography on silica to give product (20.8 g, 93.5%) as a yellow liquid.

Synthesis of Intermediate B

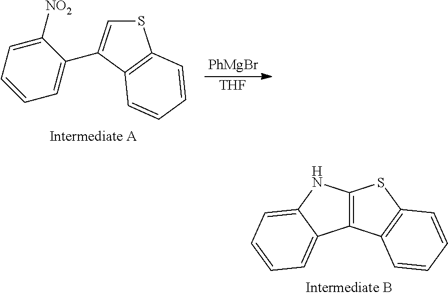 [SMILES: "PhMgBr" ; O=[N+]([O-])c1ccccc1-c1csc2ccccc12 ; c1ccc2c(c1)[nH]c1sc3ccccc3c12 ; "THF"]

PhMgBr (1 M in THF solution) (310 mL, 310.7 mmol) was slowly (0.3 mL/min) added to the mixture of Intermediate A (20 g, 88.8 mmol) and dry THF (300 mL) at 0° C. in 10 minutes. During this period, the internal temperature was closely monitored and controlled to remain below 3° C. Then the mixture was stirred at 0° C. for 5 minutes, followed by the slow and careful addition of saturated NH₄Cl aqueous solution (30 mL). The internal temperature was controlled so that it remained below 5° C. Then 50 mL of water was added and the resulting mixture was extracted with ethylacetate (3×100 mL). The combined organic layers were washed with brine (200 mL), dried over anhydrous Na₂SO₄, filtered and concentrated in vacuo. The crude product was purified by column chromatography to give product (8.9g, 51.3%) as a white solid.

Synthesis of Intermediate C

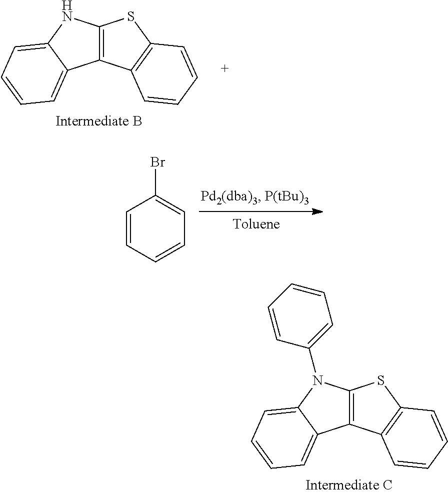 [SMILES: c1ccc2c(c1)[nH]c1sc3ccccc3c12 ; Brc1ccccc1 ; c1ccc(-n2c3ccccc3c3c4ccccc4sc32)cc1]

A mixture of 8.0 g (35.8 mmol) of Intermediate B, 7.2 g (46 mmol) of bromobenzene, 0.65 g (0.71 mmol) of Pd₂(dba)₃, 0.7 mL (0.716 mmol) of tri-tert-butylphosphine 1M in Toluene, 6.9 g (71.6 mmol) of sodium tert-butoxide, and 50 ml of toluene was degassed and placed under nitrogen gas, and then heated at 120° C. for 16 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give Intermediate C (8.0 g, 75.4%).

Synthesis of Intermediate D

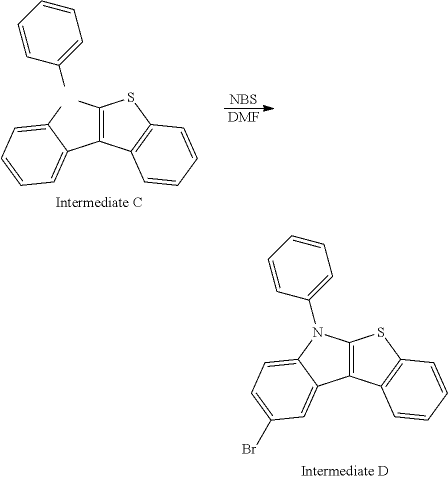

In the N₂ gas purging system, 8 g (26.7 mmole) of Intermediate C and 4.8 g (26.7 mmole) of N-bromosuccinimide were put into 80 ml of DMF, where the light was blocked out, and the mixture was stirred for 12 h. After completion of the reaction, the mixture was extracted with 250 ml of DCM and 300 ml of water. The organic layer was dried with anhydrous magnesium sulfate and the solvent was evaporated under reduced pressure. The residue was purified by column chromatography on silica to give Intermediate D (8.6 g, 86.1%) as a gray solid.

Synthesis of Intermediate E

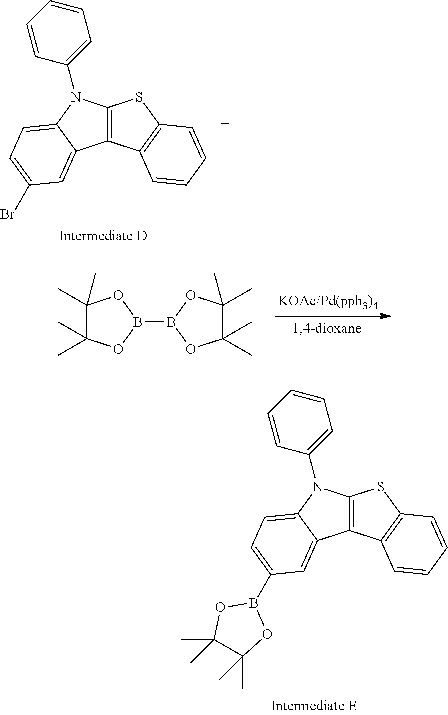

A mixture of 8 g (21.1 mmole) of Intermediate D, 7.0 g (27.4 mmol) of bis(pinacolato)diboron, 0.48 g (0.42 mmol) of tetrakis(triphenylph osphine)palladium, 6.2 g (63.3 mmol) of potassium acetate, and 60 ml of 1,4-dioxane was degassed and placed under nitrogen, and then heated at 90° C. for 16 h. After the reaction was finished, the mixture was allowed to cool to room temperature. The organic phase was separated and washed with ethyl acetate and water. After being dried with magnesium sulfate, the solvent was removed in vacuo. The residue was purified by column chromatography on silica to give product 5.0 g (56.1%) as an off-white solid.

Synthesis of compound 9

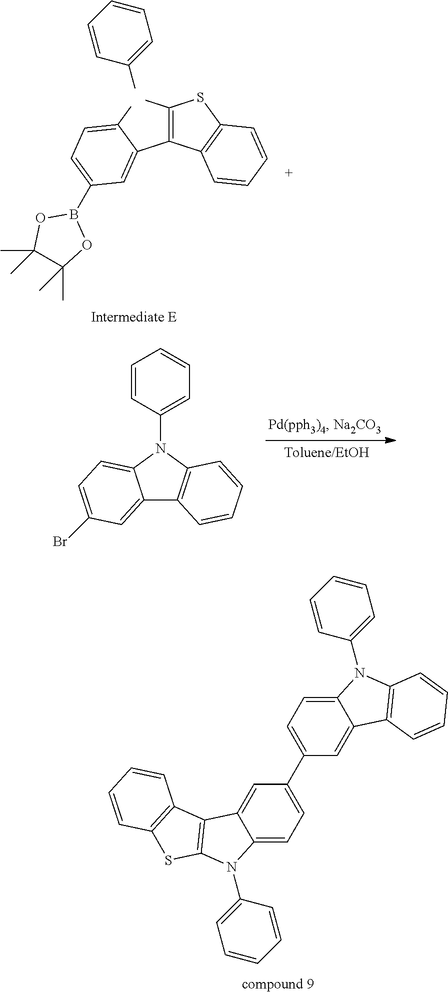 [SMILES: CC1(C)OB(c2ccc3c(c2)c2c4ccccc4sc2n3-c2ccccc2)OC1(C)C ; Brc1ccc2c(c1)c1ccccc1n2-c1ccccc1 ; c1ccc(-n2c3ccccc3c3cc(-c4ccc5c(c4)c4c6ccccc6sc4n5-c4ccccc4)ccc32)cc1]

A mixture of 5 g (11.7 mmol) of Intermediate E, 3.8 g (11.7 mmol) of 3-bromo-9-phenyl-9H-carbazole, 0.27 g (0.23 mmol) of Pd(Ph₃)₄, 11.5 ml of 2M Na₂CO₃, 50 ml of EtOH and 100 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography on silica to give of product 4.3g (68%) as white solid. MS(m/z, EI⁺):540.15.

Synthesis of Intermediate F

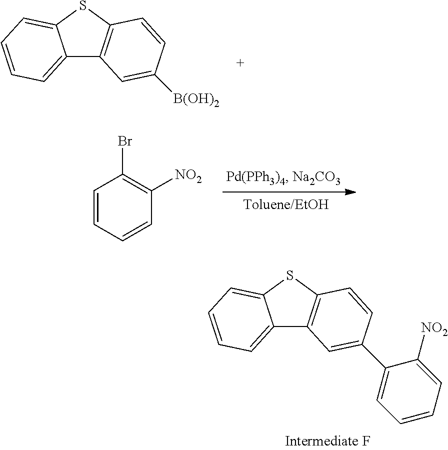 [SMILES: OB(O)c1ccc2sc3ccccc3c2c1 ; O=[N+]([O-])c1ccccc1Br ; O=[N+]([O-])c1ccccc1-c1ccc2sc3ccccc3c2c1]

A mixture of 5 g (21.9 mmol) of dibenzo[b,d]thiophen-2-ylboronic acid, 4.4 g (21.9 mmol) of 1-bromo-2-nitrobenzene, 0.5 g (0.44 mmol) of Pd(PPh₃)₄, 10 ml of 2M Na₂CO_{3(aq)}, 10 ml of EtOH, and 30 ml of toluene was degassed and placed under nitrogen, and then heated at 100° C. for 12 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give Intermediate F (5.4 g, 81%) as yellow solid.

Synthesis of Intermediate G

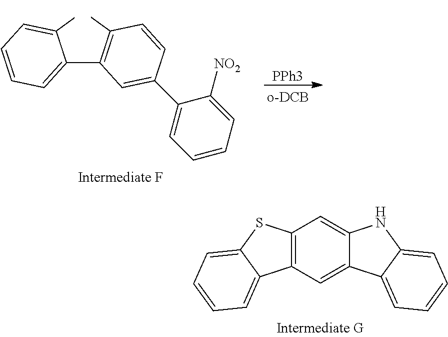 [SMILES: O=[N+]([O-])c1ccccc1-c1ccc2sc3ccccc3c2c1 ; c1ccc2c(c1)[nH]c1cc3sc4ccccc4c3cc12 ; "PPh3"]

A mixture of 5 g (16.3 mmol) of Intermediate F, 42.9 g (163.0 mmol) of Triphenylphosphine, and 250 ml of o-DCB was placed under nitrogen gas, and then heated at 180° C. for 8 hrs. After the reaction finished, the mixture was allowed to cool to room temperature. The mixture was poured into water, and then filtered to give Intermediate G (2.5 g, 56%) as pale-yellow solid.

Synthesis of Intermediate H

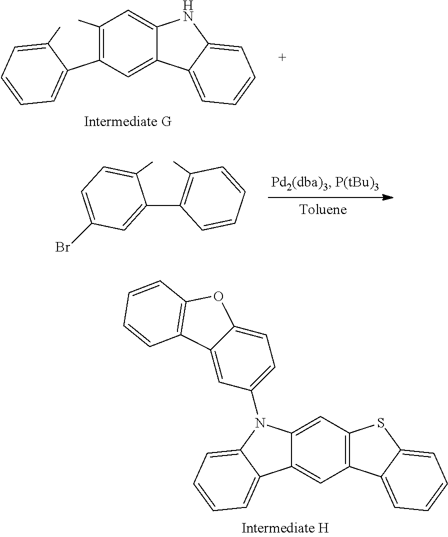 [SMILES: c1ccc2c(c1)Cc1cc3[nH]c4ccccc4c3cc1-2 ; Brc1ccc2c(c1)-c1ccccc1C2 ; c1ccc2c(c1)oc1ccc(-n3c4ccccc4c4cc5c(cc43)sc3ccccc35)cc12]

A mixture of 2.5 g (9.14 mmol) of Intermediate G, 2.5 g (10 mmol) of 2-bromodibenzofuran, 0.17 g (0.18 mmol) of Pd₂(dba)₃, 18.3 mL (18.3 mmol) of tri-tert-butylphosphine 1M in Toluene, 1.8 g (18.3 mmol) of sodium tert-butoxide, and 50 ml of toluene was degassed and placed under nitrogen gas, and then heated at 120° C. for 16 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give Intermediate H (2.9 g, 73.1%) as pale-yellow solid.

Synthesis of Intermediate I

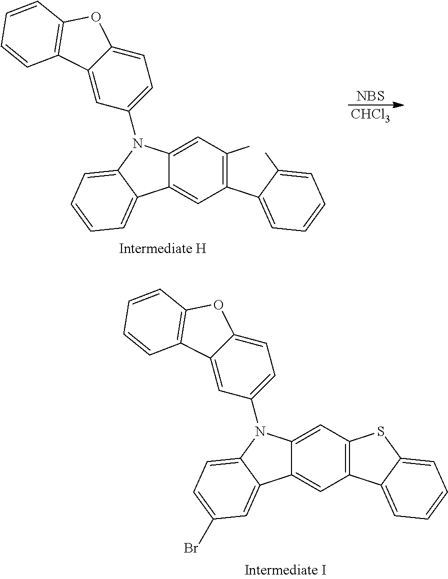 [SMILES: c1ccc2c(c1)oc1ccc(-n3c4ccccc4c4cc5c(cc43)sc3ccccc35)cc12 ; "CHCl3" ; "NBS" ; Brc1ccc2c(c1)c1cc3c(cc1n2-c1ccc2oc4ccccc4c2c1)sc1ccccc13]

In the N₂ gas purging system, 2.9 g (6.6 mmole) of Intermediate C and 6.6 g (6.6 mmole) of N-bromosuccinimide were put into 60 ml of CHCl₃, where the light was blocked out, and the mixture was stirred for 12 h. After completion of the reaction, the mixture was extracted with 100 ml of DCM and 100 ml of water. The organic layer was dried with anhydrous magnesium sulfate and the solvent was evaporated under reduced pressure. The residue was purified by column chromatography on silica to give Intermediate I (3.1 g, 90.1%) as a gray solid.

### EXAMPLE 2

Synthesis of Compound 28

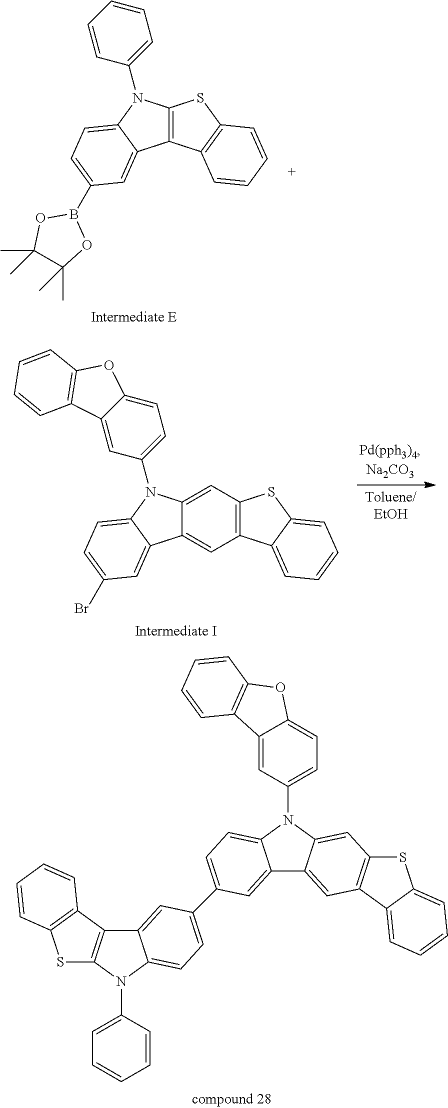 [SMILES: CC1(C)OB(c2ccc3c(c2)c2c4ccccc4sc2n3-c2ccccc2)OC1(C)C ; Brc1ccc2c(c1)c1cc3c(cc1n2-c1ccc2oc4ccccc4c2c1)sc1ccccc13 ; c1ccc(-n2c3ccc(-c4ccc5c(c4)c4cc6c(cc4n5-c4ccc5oc7ccccc7c5c4)sc4ccccc46)cc3c3c4ccccc4sc32)cc1]

A mixture of 2.46 g (5.78 mmol) of Intermediate E, 3.0 g (5.78 mmol) of Intermediate I, 0.12 g (0.11 mmol) of Pd(Ph₃)₄, 6 ml of 2M Na₂CO₃, 20 ml of EtOH and 60 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography on silica to give of product 2.8 g (66.3%) as off-white solid. MS(m/z, EI⁺):736.15.

### EXAMPLE 3

Synthesis of Compound 44

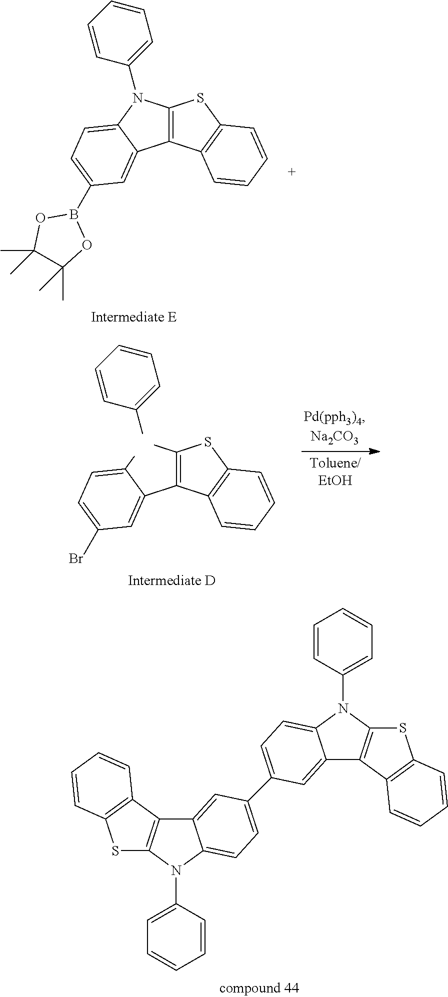 [SMILES: CC1(C)OB(c2ccc3c(c2)c2c4ccccc4sc2n3-c2ccccc2)OC1(C)C ; Brc1ccc2c(c1)c1c3ccccc3sc1n2-c1ccccc1 ; c1ccc(-n2c3ccc(-c4ccc5c(c4)c4c6ccccc6sc4n5-c4ccccc4)cc3c3c4ccccc4sc32)cc1]

A mixture of 3.0 g (7.05 mmol) of Intermediate E, 2.67 g (7.05 mmol) of Intermediate D, 0.16 g (0.14 mmol) of Pd(Ph₃)₄, 6 ml of 2M Na₂CO₃, 20 ml of EtOH and 60 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography on silica to give of product 4.1 g (88.1%) as off-white solid. MS(m/z, EI⁺):596.16.

### EXAMPLE 4-23

A series of intermediates and the product compounds are synthesized analogously, as follows.

| Ex. | Intermediate I | Intermediate II | Product | Yield |
|---|---|---|---|---|
| 4 | 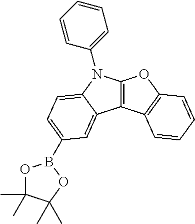 [SMILES: CC1(C)OB(c2ccc3c(c2)c2c4ccccc4oc2n3-c2ccccc2)OC1(C)C] | 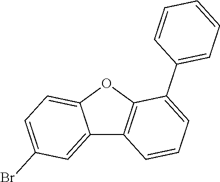 [SMILES: Brc1ccc2oc3c(-c4ccccc4)cccc3c2c1] | 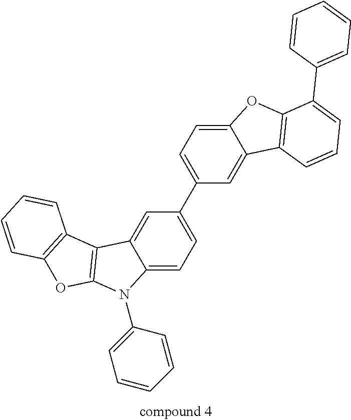 [SMILES: c1ccc(-c2cccc3c2oc2ccc(-c4ccc5c(c4)c4c6ccccc6oc4n5-c4ccccc4)cc23)cc1] | 69% |
| 5 | 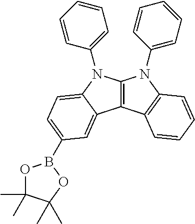 [SMILES: CC1(C)OB(c2ccc3c(c2)c2c4ccccc4n(-c4ccccc4)c2n3-c2ccccc2)OC1(C)C] | 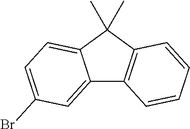 [SMILES: CC1(C)c2ccccc2-c2cc(Br)ccc21] | 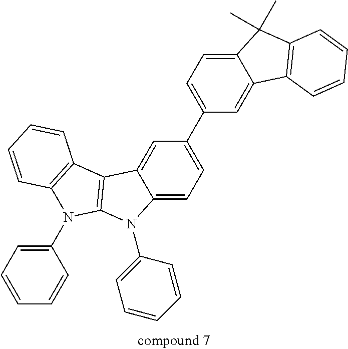 [SMILES: CC1(C)c2ccccc2-c2cc(-c3ccc4c(c3)c3c5ccccc5n(-c5ccccc5)c3n4-c3ccccc3)ccc21] | 54% |
| 6 | 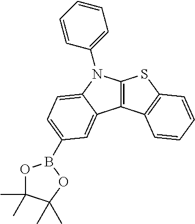 [SMILES: CC1(C)OB(c2ccc3c(c2)c2c4ccccc4sc2n3-c2ccccc2)OC1(C)C] | 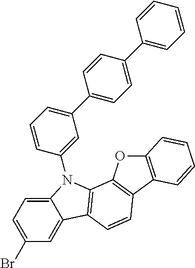 [SMILES: Brc1ccc2c(c1)c1ccc3c4ccccc4oc3c1n2-c1cccc(-c2ccc(-c3ccccc3)cc2)c1] | 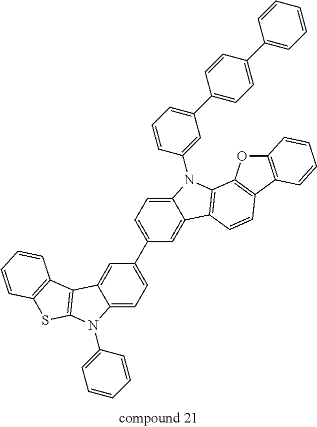 [SMILES: c1ccc(-c2ccc(-c3cccc(-n4c5ccc(-c6ccc7c(c6)c6c8ccccc8sc6n7-c6ccccc6)cc5c5ccc6c7ccccc7oc6c54)c3)cc2)cc1] | 48% |
| 7 | 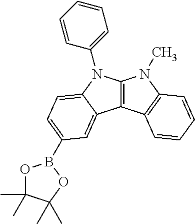 [SMILES: Cn1c2ccccc2c2c3cc(B4OC(C)(C)C(C)(C)O4)ccc3n(-c3ccccc3)c21] | 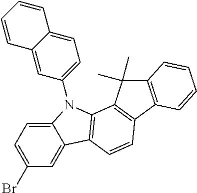 [SMILES: CC1(C)c2ccccc2-c2ccc3c4cc(Br)ccc4n(-c4ccc5ccccc5c4)c3c21] | 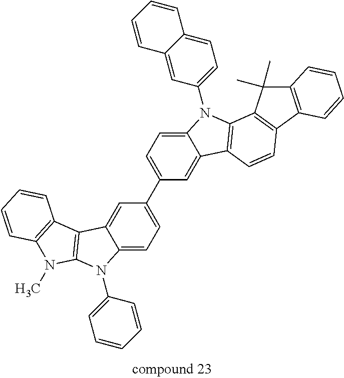 [SMILES: Cn1c2ccccc2c2c3cc(-c4ccc5c(c4)c4ccc6c(c4n5-c4ccc5ccccc5c4)C(C)(C)c4ccccc4-6)ccc3n(-c3ccccc3)c21] | 62% |
| 8 | 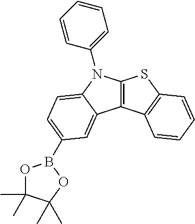 [SMILES: CC1(C)OB(c2ccc3c(c2)c2c4ccccc4sc2n3-c2ccccc2)OC1(C)C] | 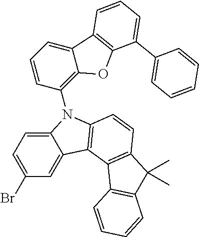 [SMILES: CC1(C)c2ccccc2-c2c1ccc1c2c2cc(Br)ccc2n1-c1cccc2c1oc1c(-c3ccccc3)cccc12] | 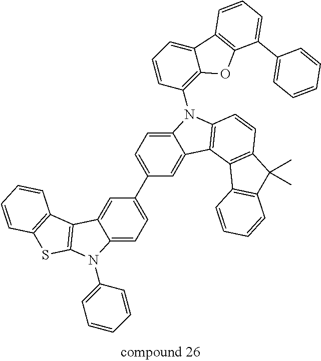 [SMILES: CC1(C)c2ccccc2-c2c1ccc1c2c2cc(-c3ccc4c(c3)c3c5ccccc5sc3n4-c3ccccc3)ccc2n1-c1cccc2c1oc1c(-c3ccccc3)cccc12] | 57% |
| 9 | 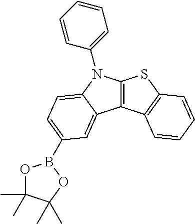 [SMILES: CC1(C)OB(c2ccc3c(c2)c2c4ccccc4sc2n3-c2ccccc2)OC1(C)C] | 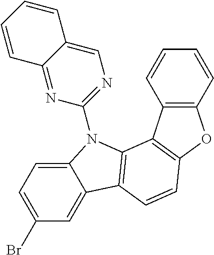 [SMILES: Brc1ccc2c(c1)c1ccc3oc4ccccc4c3c1n2-c1ncc2ccccc2n1] | 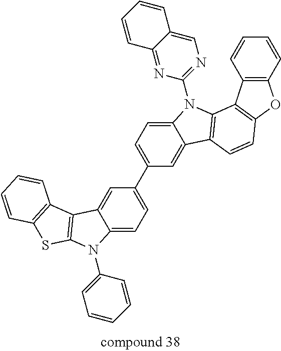 [SMILES: c1ccc(-n2c3ccc(-c4ccc5c(c4)c4ccc6oc7ccccc7c6c4n5-c4ncc5ccccc5n4)cc3c3c4ccccc4sc32)cc1] | 64% |
| 10 | 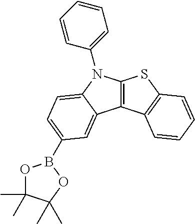 [SMILES: CC1(C)OB(c2ccc3c(c2)c2c4ccccc4sc2n3-c2ccccc2)OC1(C)C] | 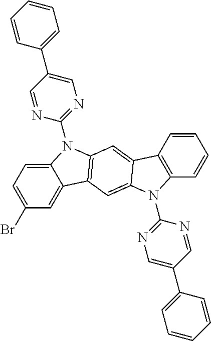 [SMILES: Brc1ccc2c(c1)c1cc3c(cc1n2-c1ncc(-c2ccccc2)cn1)c1ccccc1n3-c1ncc(-c2ccccc2)cn1] | 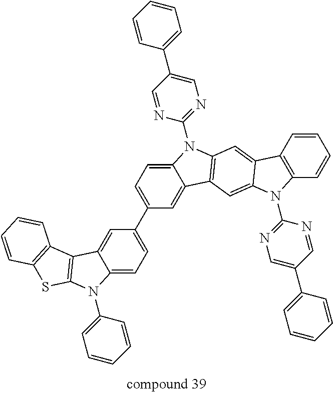 [SMILES: c1ccc(-c2cnc(-n3c4ccccc4c4cc5c(cc43)c3cc(-c4ccc6c(c4)c4c7ccccc7sc4n6-c4ccccc4)ccc3n5-c3ncc(-c4ccccc4)cn3)nc2)cc1] | 62% |
| 11 | 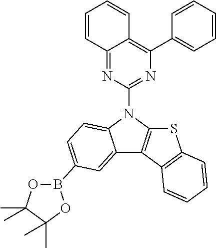 [SMILES: CC1(C)OB(c2ccc3c(c2)c2c4ccccc4sc2n3-c2nc(-c3ccccc3)c3ccccc3n2)OC1(C)C] | 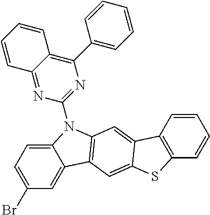 [SMILES: Brc1ccc2c(c1)c1cc3sc4ccccc4c3cc1n2-c1nc(-c2ccccc2)c2ccccc2n1] | 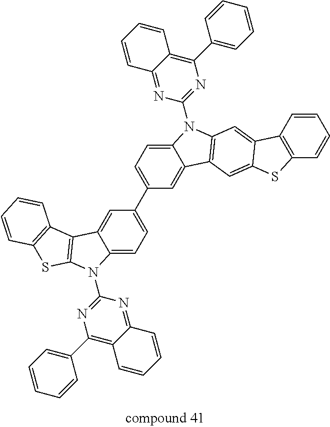 [SMILES: c1ccc(-c2nc(-n3c4ccc(-c5ccc6c(c5)c5c7ccccc7sc5n6-c5nc(-c6ccccc6)c6ccccc6n5)cc4c4cc5sc6ccccc6c5cc43)nc3ccccc23)cc1] | 51% |
| 12 | 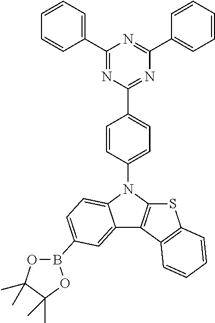 [SMILES: CC1(C)OB(c2ccc3c(c2)c2c4ccccc4sc2n3-c2ccc(-c3nc(-c4ccccc4)nc(-c4ccccc4)n3)cc2)OC1(C)C] | 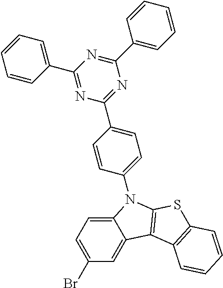 [SMILES: Brc1ccc2c(c1)c1c3ccccc3sc1n2-c1ccc(-c2nc(-c3ccccc3)nc(-c3ccccc3)n2)cc1] | 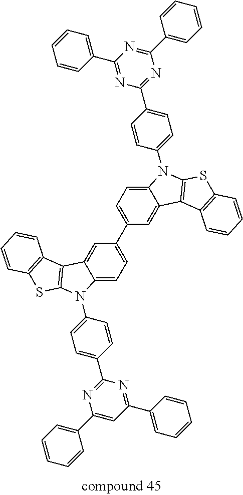 [SMILES: c1ccc(-c2cc(-c3ccccc3)nc(-c3ccc(-n4c5ccc(-c6ccc7c(c6)c6c8ccccc8sc6n7-c6ccc(-c7nc(-c8ccccc8)nc(-c8ccccc8)n7)cc6)cc5c5c6ccccc6sc54)cc3)n2)cc1] | 58% |
| 13 | 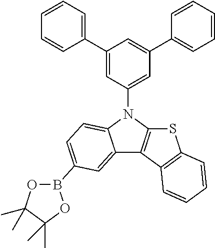 [SMILES: CC1(C)OB(c2ccc3c(c2)c2c4ccccc4sc2n3-c2cc(-c3ccccc3)cc(-c3ccccc3)c2)OC1(C)C] | 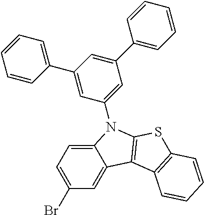 [SMILES: Brc1ccc2c(c1)c1c3ccccc3sc1n2-c1cc(-c2ccccc2)cc(-c2ccccc2)c1] | 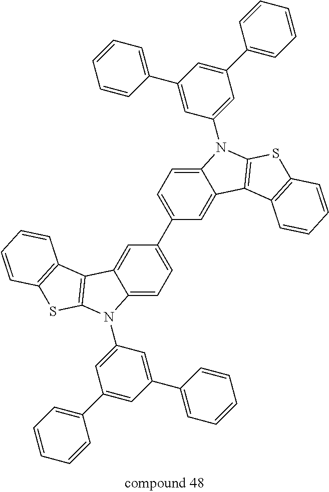 [SMILES: c1ccc(-c2cc(-c3ccccc3)cc(-n3c4ccc(-c5ccc6c(c5)c5c7ccccc7sc5n6-c5cc(-c6ccccc6)cc(-c6ccccc6)c5)cc4c4c5ccccc5sc43)c2)cc1] | 65% |
| 14 | 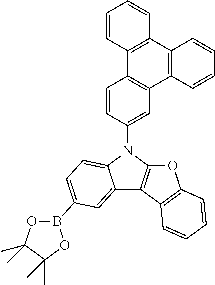 [SMILES: CC1(C)OB(c2ccc3c(c2)c2c4ccccc4oc2n3-c2ccc3c4ccccc4c4ccccc4c3c2)OC1(C)C] | 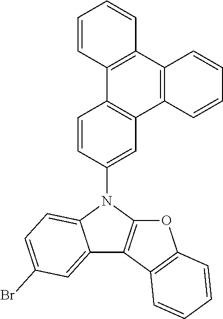 [SMILES: Brc1ccc2c(c1)c1c3ccccc3oc1n2-c1ccc2c3ccccc3c3ccccc3c2c1] | 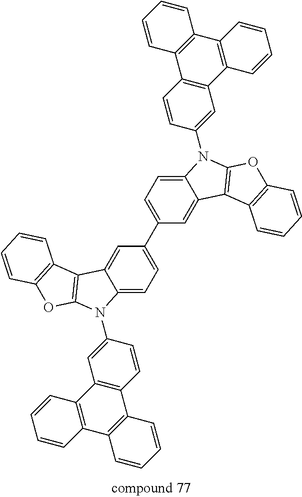 [SMILES: c1ccc2c(c1)oc1c2c2cc(-c3ccc4c(c3)c3c5ccccc5oc3n4-c3ccc4c5ccccc5c5ccccc5c4c3)ccc2n1-c1ccc2c3ccccc3c3ccccc3c2c1] | 49% |
| 15 | 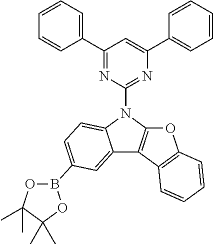 [SMILES: CC1(C)OB(c2ccc3c(c2)c2c4ccccc4oc2n3-c2nc(-c3ccccc3)cc(-c3ccccc3)n2)OC1(C)C] | 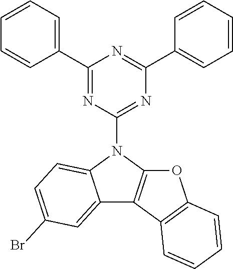 [SMILES: Brc1ccc2c(c1)c1c3ccccc3oc1n2-c1nc(-c2ccccc2)nc(-c2ccccc2)n1] | 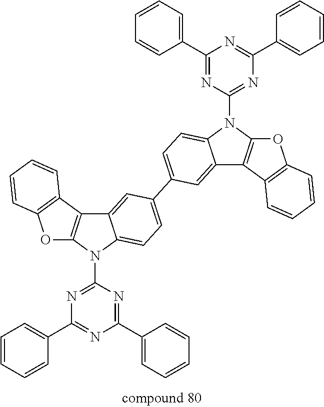 [SMILES: c1ccc(-c2nc(-c3ccccc3)nc(-n3c4ccc(-c5ccc6c(c5)c5c7ccccc7oc5n6-c5nc(-c6ccccc6)nc(-c6ccccc6)n5)cc4c4c5ccccc5oc43)n2)cc1] | 66% |
| 16 | 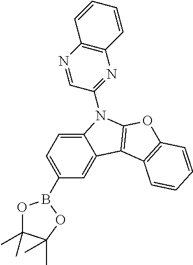 [SMILES: CC1(C)OB(c2ccc3c(c2)c2c4ccccc4oc2n3-c2cnc3ccccc3n2)OC1(C)C] | 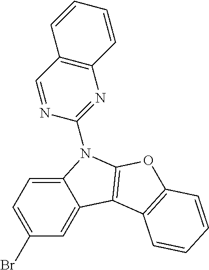 [SMILES: Brc1ccc2c(c1)c1c3ccccc3oc1n2-c1ncc2ccccc2n1] | 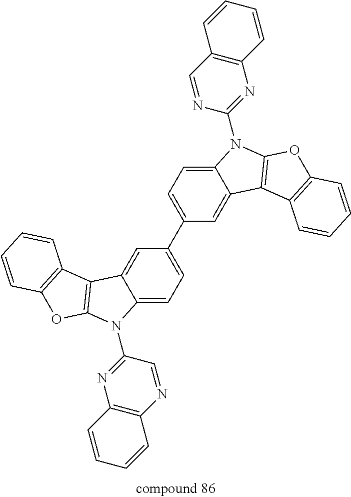 [SMILES: c1ccc2nc(-n3c4ccc(-c5ccc6c(c5)c5c7ccccc7oc5n6-c5cnc6ccccc6n5)cc4c4c5ccccc5oc43)ncc2c1] | 51% |
| 17 | 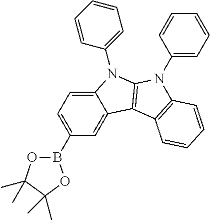 [SMILES: CC1(C)OB(c2ccc3c(c2)c2c4ccccc4n(-c4ccccc4)c2n3-c2ccccc2)OC1(C)C] | 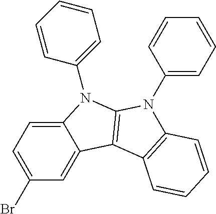 [SMILES: Brc1ccc2c(c1)c1c3ccccc3n(-c3ccccc3)c1n2-c1ccccc1] | 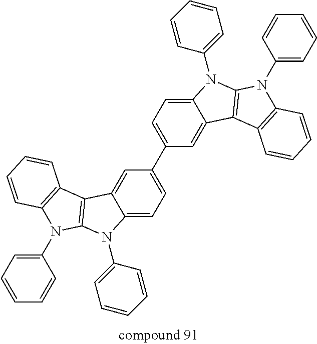 [SMILES: c1ccc(-n2c3ccccc3c3c4cc(-c5ccc6c(c5)c5c7ccccc7n(-c7ccccc7)c5n6-c5ccccc5)ccc4n(-c4ccccc4)c32)cc1] | 45% |
| 18 | 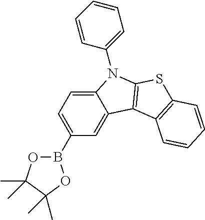 [SMILES: CC1(C)OB(c2ccc3c(c2)c2c4ccccc4sc2n3-c2ccccc2)OC1(C)C] | 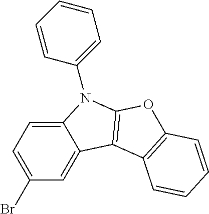 [SMILES: Brc1ccc2c(c1)c1c3ccccc3oc1n2-c1ccccc1] | 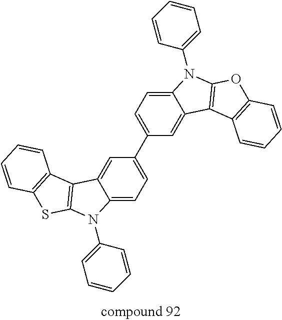 [SMILES: c1ccc(-n2c3ccc(-c4ccc5c(c4)c4c6ccccc6sc4n5-c4ccccc4)cc3c3c4ccccc4oc32)cc1] | 70% |
| 19 | 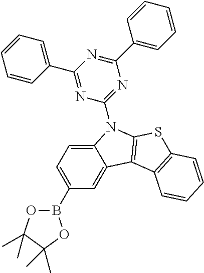 [SMILES: CC1(C)OB(c2ccc3c(c2)c2c4ccccc4sc2n3-c2nc(-c3ccccc3)nc(-c3ccccc3)n2)OC1(C)C] | 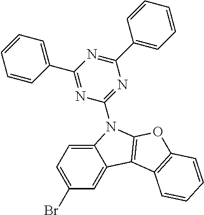 [SMILES: Brc1ccc2c(c1)c1c3ccccc3oc1n2-c1nc(-c2ccccc2)nc(-c2ccccc2)n1] | 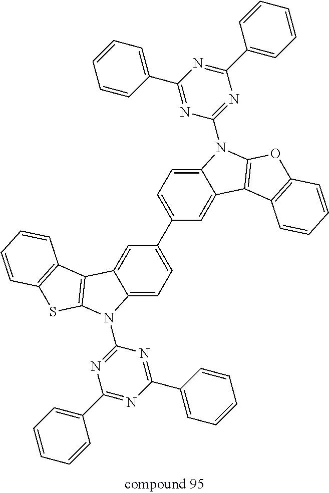 [SMILES: c1ccc(-c2nc(-c3ccccc3)nc(-n3c4ccc(-c5ccc6c(c5)c5c7ccccc7sc5n6-c5nc(-c6ccccc6)nc(-c6ccccc6)n5)cc4c4c5ccccc5oc43)n2)cc1] | 51% |
| 20 | 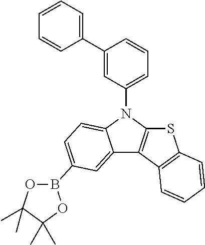 [SMILES: CC1(C)OB(c2ccc3c(c2)c2c4ccccc4sc2n3-c2cccc(-c3ccccc3)c2)OC1(C)C] | 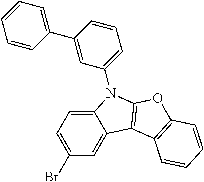 [SMILES: Brc1ccc2c(c1)c1c3ccccc3oc1n2-c1cccc(-c2ccccc2)c1] | 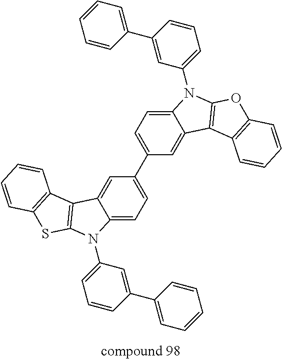 [SMILES: c1ccc(-c2cccc(-n3c4ccc(-c5ccc6c(c5)c5c7ccccc7sc5n6-c5cccc(-c6ccccc6)c5)cc4c4c5ccccc5oc43)c2)cc1] | 68% |
| 21 | 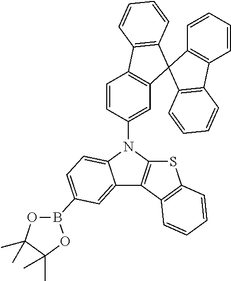 [SMILES: CC1(C)OB(c2ccc3c(c2)c2c4ccccc4sc2n3-c2ccc3c(c2)C2(c4ccccc4-c4ccccc42)c2ccccc2-3)OC1(C)C] | 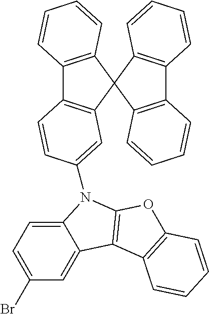 [SMILES: Brc1ccc2c(c1)c1c3ccccc3oc1n2-c1ccc2c(c1)C1(c3ccccc3-c3ccccc31)c1ccccc1-2] | 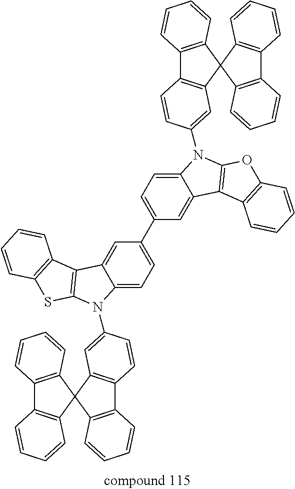 [SMILES: c1ccc2c(c1)-c1ccccc1C21c2ccccc2-c2ccc(-n3c4ccc(-c5ccc6c(c5)c5c7ccccc7sc5n6-c5ccc6c(c5)C5(c7ccccc7-c7ccccc75)c5ccccc5-6)cc4c4c5ccccc5oc43)cc21] | 33% |
| 22 | 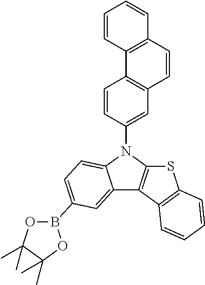 [SMILES: CC1(C)OB(c2ccc3c(c2)c2c4ccccc4sc2n3-c2ccc3c(ccc4ccccc43)c2)OC1(C)C] | 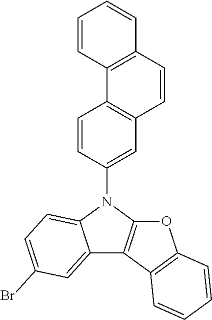 [SMILES: Brc1ccc2c(c1)c1c3ccccc3oc1n2-c1ccc2c(ccc3ccccc32)c1] | 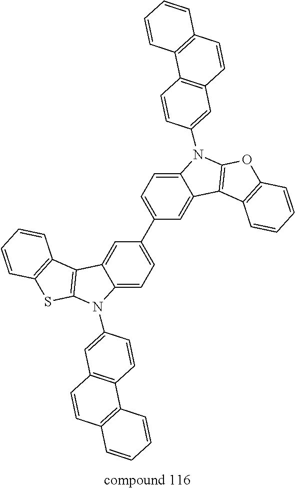 [SMILES: c1ccc2c(c1)ccc1cc(-n3c4ccc(-c5ccc6c(c5)c5c7ccccc7sc5n6-c5ccc6c(ccc7ccccc76)c5)cc4c4c5ccccc5oc43)ccc12] | 48% |
| 23 | 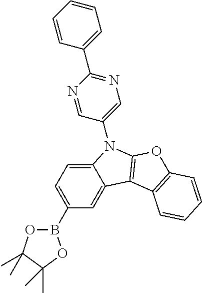 [SMILES: CC1(C)OB(c2ccc3c(c2)c2c4ccccc4oc2n3-c2cnc(-c3ccccc3)nc2)OC1(C)C] | 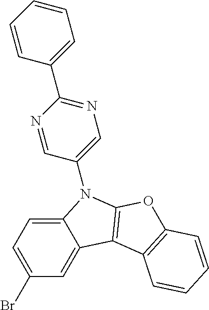 [SMILES: Brc1ccc2c(c1)c1c3ccccc3oc1n2-c1cnc(-c2ccccc2)nc1] | 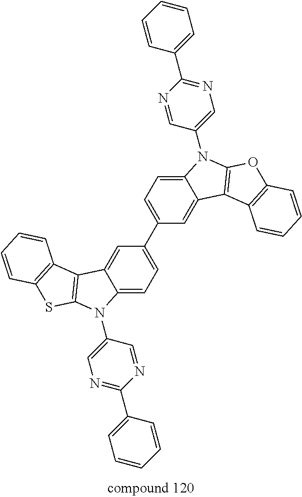 [SMILES: c1ccc(-c2ncc(-n3c4ccc(-c5ccc6c(c5)c5c7ccccc7sc5n6-c5cnc(-c6ccccc6)nc5)cc4c4c5ccccc5oc43)cn2)cc1] | 69% |

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

What is claimed is:

1. An organic compound of formula (1): 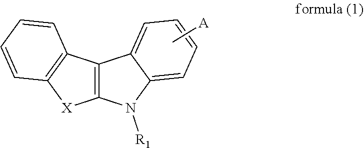
wherein A represents mono to the maximum allowable substitution;
wherein each A comprises at least one chemical group selected from the group consisting of 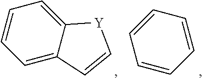
and combinations thereof;
wherein each Y is divalent bridge selected from the group consisting from O, S, CR₇R₈ and NR₉;
wherein X is a divalent bridge selected from the group consisting of O, S and NR₆; and
wherein R₁, R₆, R₇, R₈, and R₉ are independently hydrogen or a substituent selected from the group consisting of alkyl, aralkyl, heteroaryl, deuterium, halide, cycloalkyl, heteroalkyl, arylalkyl, alkoxy, aryloxy, amino, silyl, alkenyl, cycloalkenyl, heteroalkenyl, alkynyl, acyl, carbonyl, carboxylic acids, ester, nitrile, isonitrile, sulfanyl, sulfinyl, sulfonyl, phosphino, and combinations thereof.

2. The organic compound according to claim 1, wherein A has the formula (6): 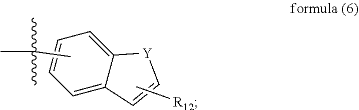
wherein R₁₂ represents mono to the maximum allowable substitution; and
wherein each R₁₂ is hydrogen or a substituent selected from the group consisting of alkyl, aralkyl, aralkyl, heteroaryl, deuterium, halide, cycloalkyl, heteroalkyl, arylalkyl, alkoxy, aryloxy, amino, silyl, alkenyl, cycloalkenyl, heteroalkenyl, alkynyl, acyl, carbonyl, carboxylic acids, ester, nitrile, isonitrile, sulfanyl, sulfinyl, sulfonyl, phosphino, and combinations thereof; and
wherein two or more R₁₂ substituents are optionally joined or fused into a ring.

3. The organic compound according to claim 1, wherein A has one of the formula (2) to formula (5): 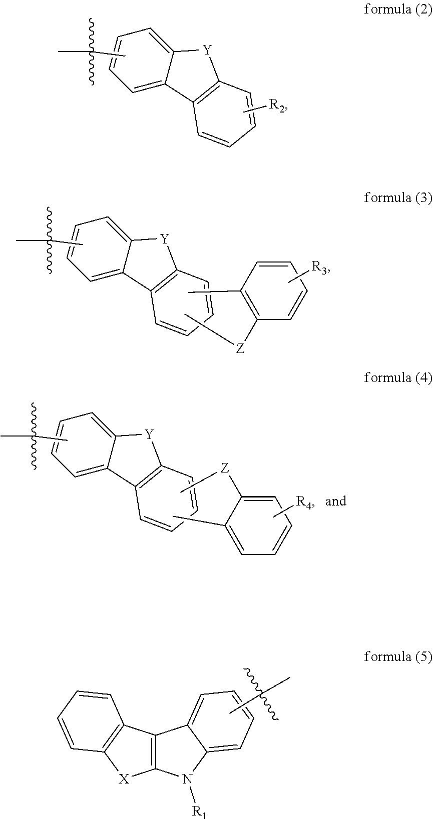
wherein each X is a divalent bridge selected from the group consisting of O, S and NR₆;
each Y is divalent bridge selected from the group consisting from O, S, CR₇R₈ and NR₉;
each Z is divalent bridge selected from the group consisting from O, S, CR₁₀R₁₁ and NR₁₂; and
R₁ to R₁₁ are independently selected from the group consisting of hydrogen, alkyl having 1 to 30 carbon atoms, aryl having 6 to 30 carbon atoms, aralkyl having 6 to 30 carbon atoms, heteroaryl having 6 to 30 carbon atoms, and combinations thereof.

4. The organic compound according to claim 1, wherein at least one of R₁, R₅, R₆, R₉ and R₁₂ is selected from the group consisting of triphenylene, carbazole, dibenzothiophene, dibenzofuran, dibenzoselenophene, phenyl, pyridine, pyrimidine, pyrazine, triazine, diazine, benzimidazole, imidazole, quinolone, isoquinolone, quinoazoline, quinoxaline, phenanthrene, biphenyl, terphenyl, o-terphenyl, m-terphenyl, p-terphenyl, and combinations thereof.

5. The organic compound according to claim 1, wherein the organic compound has one of the following formula (1-1) to formula (1-4):

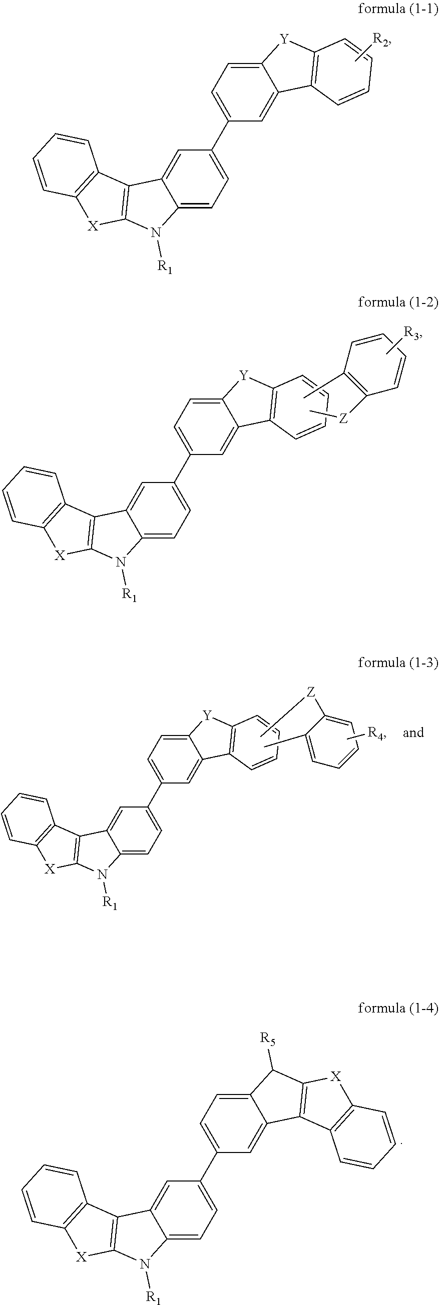

6. The organic compound according to claim 1, wherein R₁, R₅, R₆, R₉ and R₁₂ represents one of the following substituents:

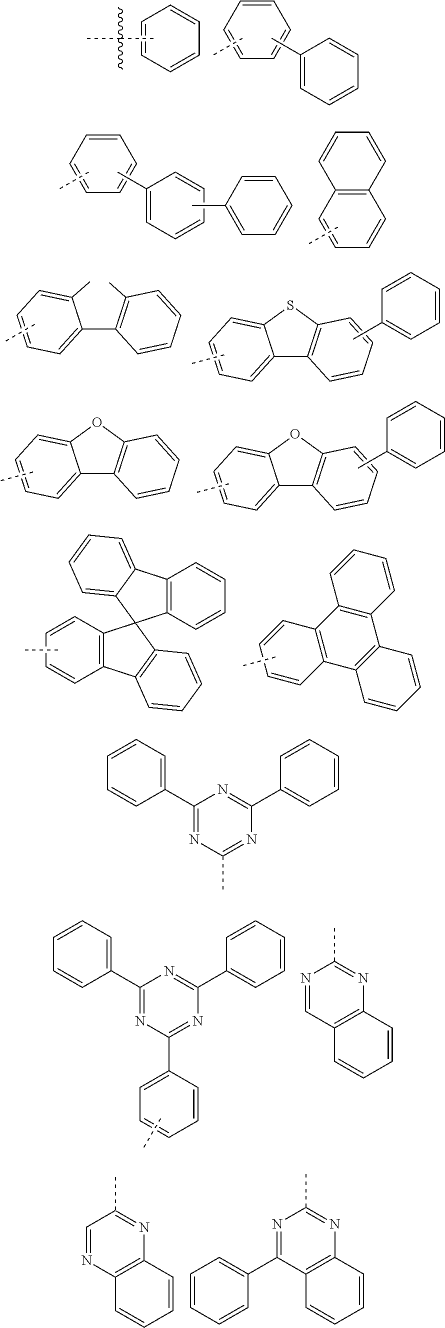

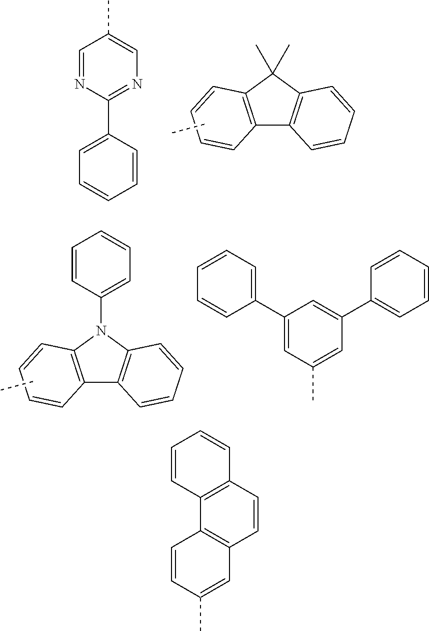

7. The organic compound according to claim 1, wherein the organic compound is selected from the group consisting of:

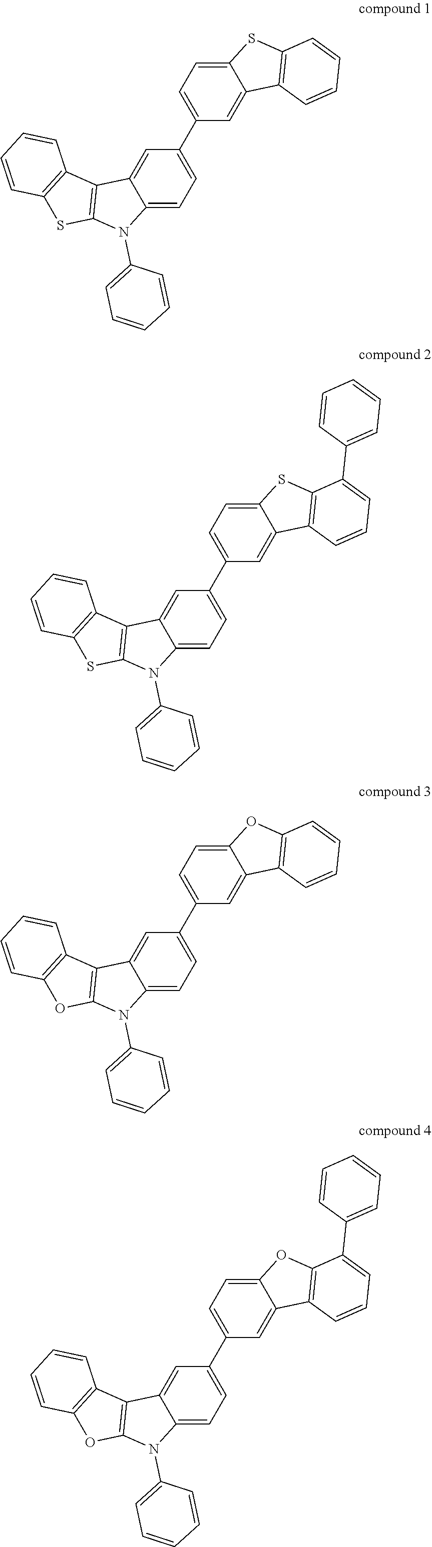 [SMILES: c1ccc(-n2c3ccc(-c4ccc5sc6ccccc6c5c4)cc3c3c4ccccc4sc32)cc1 ; c1ccc(-c2cccc3c2sc2ccc(-c4ccc5c(c4)c4c6ccccc6sc4n5-c4ccccc4)cc23)cc1 ; c1ccc(-n2c3ccc(-c4ccc5oc6ccccc6c5c4)cc3c3c4ccccc4oc32)cc1 ; c1ccc(-c2cccc3c2oc2ccc(-c4ccc5c(c4)c4c6ccccc6oc4n5-c4ccccc4)cc23)cc1]

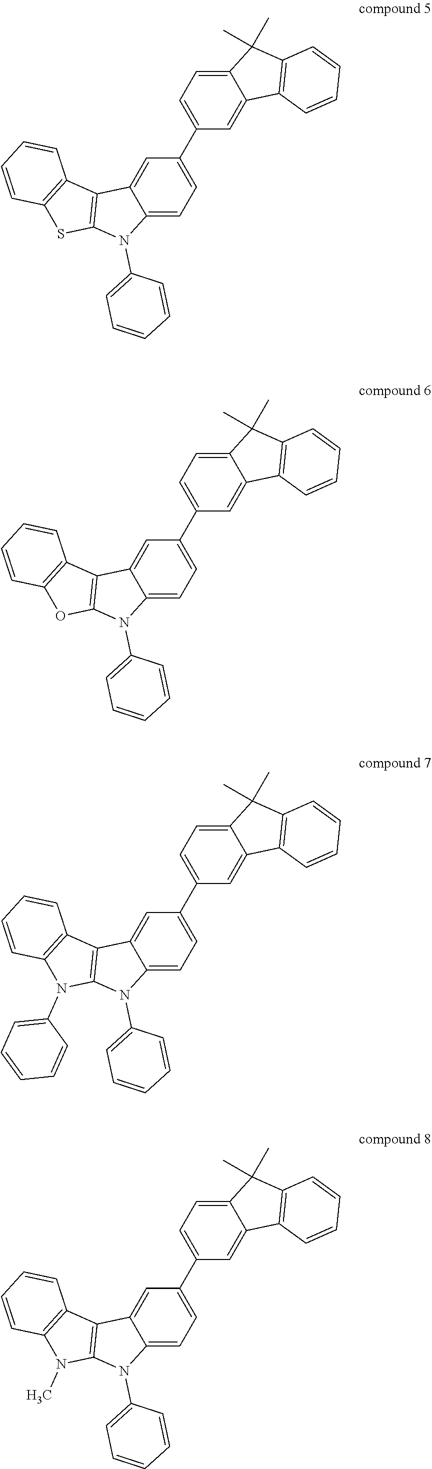 [SMILES: CC1(C)c2ccccc2-c2cc(-c3ccc4c(c3)c3c5ccccc5sc3n4-c3ccccc3)ccc21 ; CC1(C)c2ccccc2-c2cc(-c3ccc4c(c3)c3c5ccccc5oc3n4-c3ccccc3)ccc21 ; CC1(C)c2ccccc2-c2cc(-c3ccc4c(c3)c3c5ccccc5n(-c5ccccc5)c3n4-c3ccccc3)ccc21 ; Cn1c2ccccc2c2c3cc(-c4ccc5c(c4)-c4ccccc4C5(C)C)ccc3n(-c3ccccc3)c21]

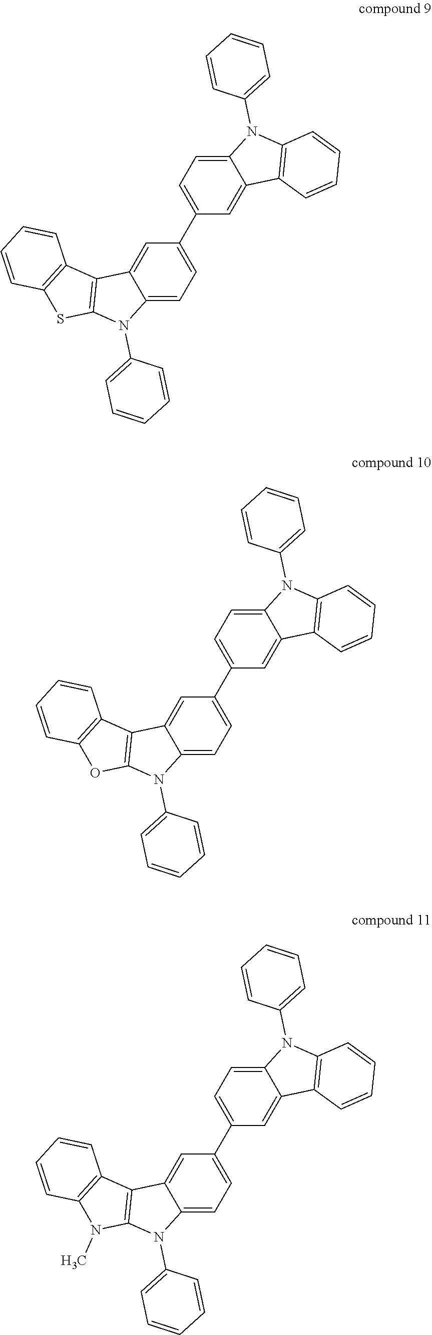 [SMILES: c1ccc(-n2c3ccccc3c3cc(-c4ccc5c(c4)c4c6ccccc6sc4n5-c4ccccc4)ccc32)cc1 ; c1ccc(-n2c3ccccc3c3cc(-c4ccc5c(c4)c4c6ccccc6oc4n5-c4ccccc4)ccc32)cc1 ; Cn1c2ccccc2c2c3cc(-c4ccc5c(c4)c4ccccc4n5-c4ccccc4)ccc3n(-c3ccccc3)c21]

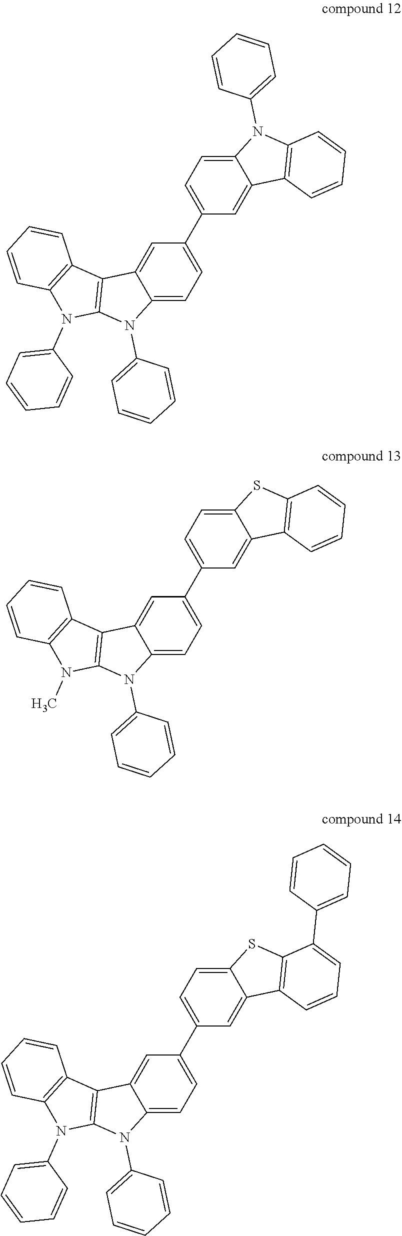 [SMILES: c1ccc(-n2c3ccccc3c3cc(-c4ccc5c(c4)c4c6ccccc6n(-c6ccccc6)c4n5-c4ccccc4)ccc32)cc1 ; Cn1c2ccccc2c2c3cc(-c4ccc5sc6ccccc6c5c4)ccc3n(-c3ccccc3)c21 ; c1ccc(-c2cccc3c2sc2ccc(-c4ccc5c(c4)c4c6ccccc6n(-c6ccccc6)c4n5-c4ccccc4)cc23)cc1]

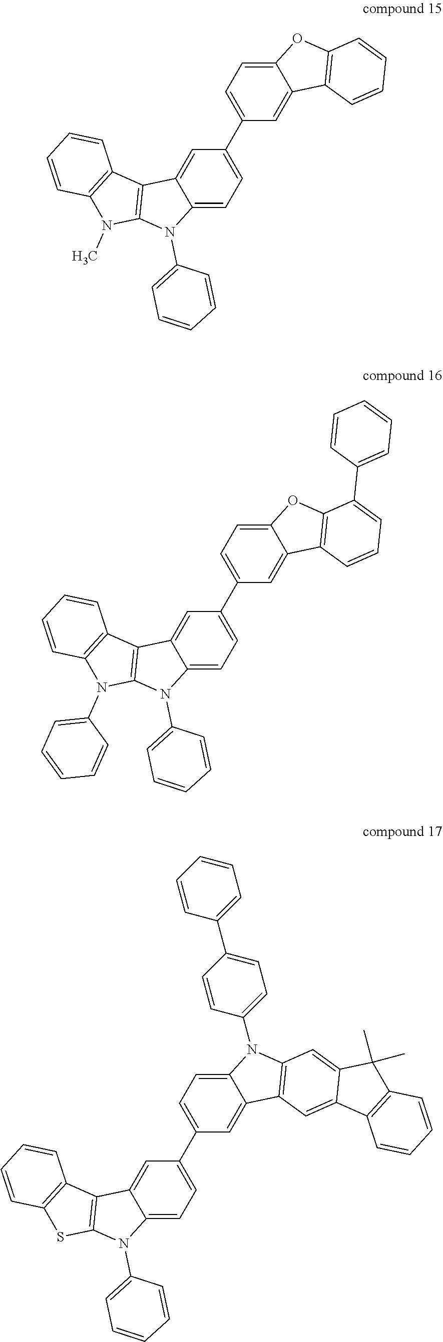 [SMILES: Cn1c2ccccc2c2c3cc(-c4ccc5oc6ccccc6c5c4)ccc3n(-c3ccccc3)c21 ; c1ccc(-c2cccc3c2oc2ccc(-c4ccc5c(c4)c4c6ccccc6n(-c6ccccc6)c4n5-c4ccccc4)cc23)cc1 ; CC1(C)c2ccccc2-c2cc3c4cc(-c5ccc6c(c5)c5c7ccccc7sc5n6-c5ccccc5)ccc4n(-c4ccc(-c5ccccc5)cc4)c3cc21]

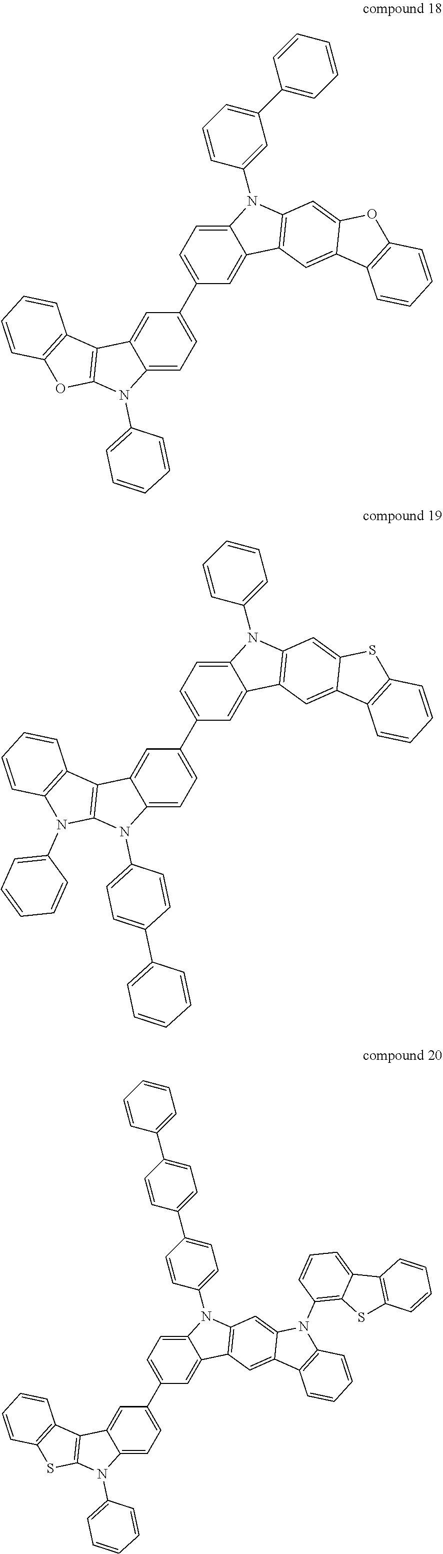 [SMILES: c1ccc(-c2cccc(-n3c4ccc(-c5ccc6c(c5)c5c7ccccc7oc5n6-c5ccccc5)cc4c4cc5c(cc43)oc3ccccc35)c2)cc1 ; c1ccc(-c2ccc(-n3c4ccc(-c5ccc6c(c5)c5cc7c(cc5n6-c5ccccc5)sc5ccccc57)cc4c4c5ccccc5n(-c5ccccc5)c43)cc2)cc1 ; c1ccc(-c2ccc(-c3ccc(-n4c5ccc(-c6ccc7c(c6)c6c8ccccc8sc6n7-c6ccccc6)cc5c5cc6c7ccccc7n(-c7cccc8c7sc7ccccc78)c6cc54)cc3)cc2)cc1]

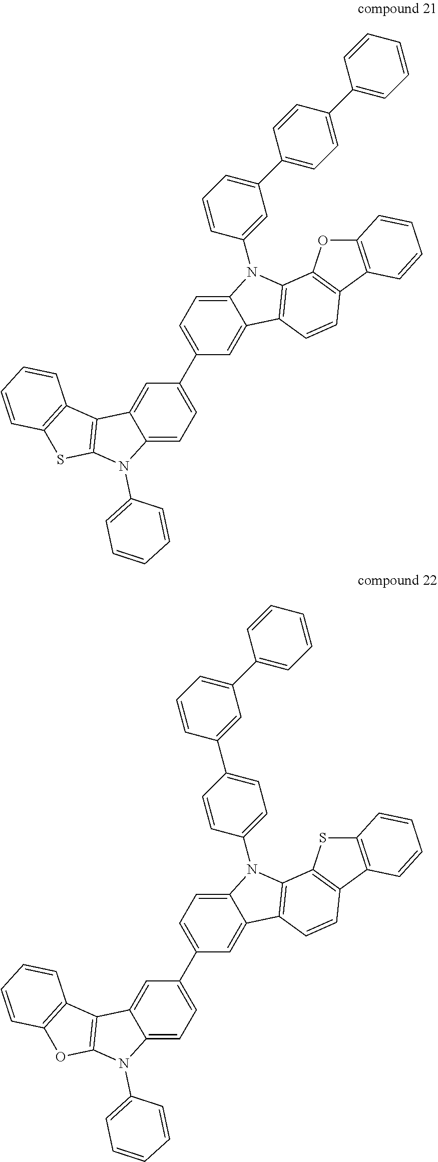 [SMILES: c1ccc(-c2ccc(-c3cccc(-n4c5ccc(-c6ccc7c(c6)c6c8ccccc8sc6n7-c6ccccc6)cc5c5ccc6c7ccccc7oc6c54)c3)cc2)cc1 ; c1ccc(-c2cccc(-c3ccc(-n4c5ccc(-c6ccc7c(c6)c6c8ccccc8oc6n7-c6ccccc6)cc5c5ccc6c7ccccc7sc6c54)cc3)c2)cc1]

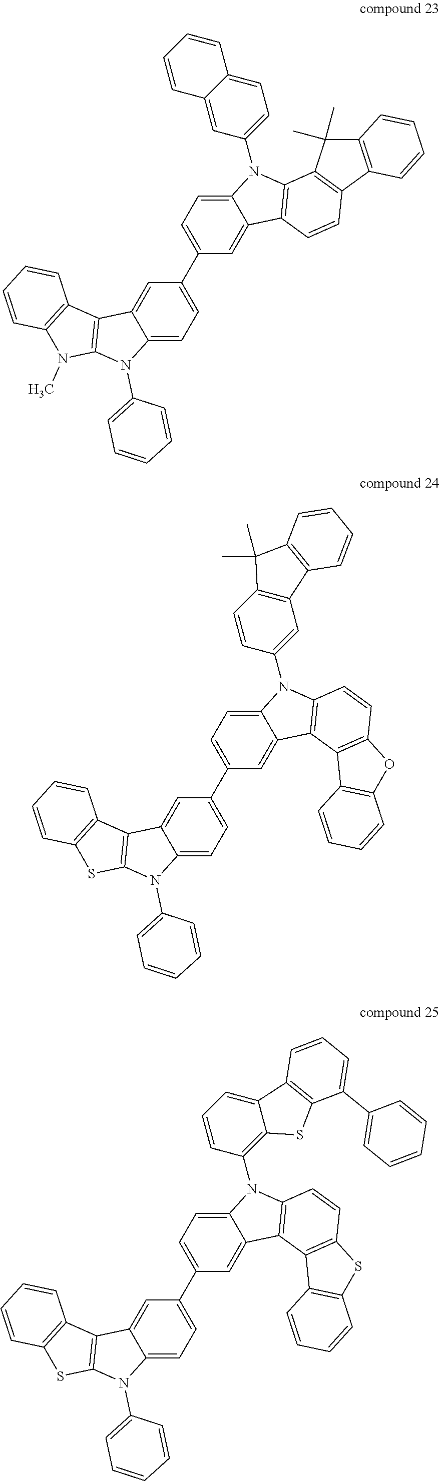 [SMILES: Cn1c2ccccc2c2c3cc(-c4ccc5c(c4)c4ccc6c(c4n5-c4ccc5ccccc5c4)C(C)(C)c4ccccc4-6)ccc3n(-c3ccccc3)c21 ; CC1(C)c2ccccc2-c2cc(-n3c4ccc(-c5ccc6c(c5)c5c7ccccc7sc5n6-c5ccccc5)cc4c4c5c(ccc43)oc3ccccc35)ccc21 ; c1ccc(-c2cccc3c2sc2c(-n4c5ccc(-c6ccc7c(c6)c6c8ccccc8sc6n7-c6ccccc6)cc5c5c6c(ccc54)sc4ccccc46)cccc23)cc1]

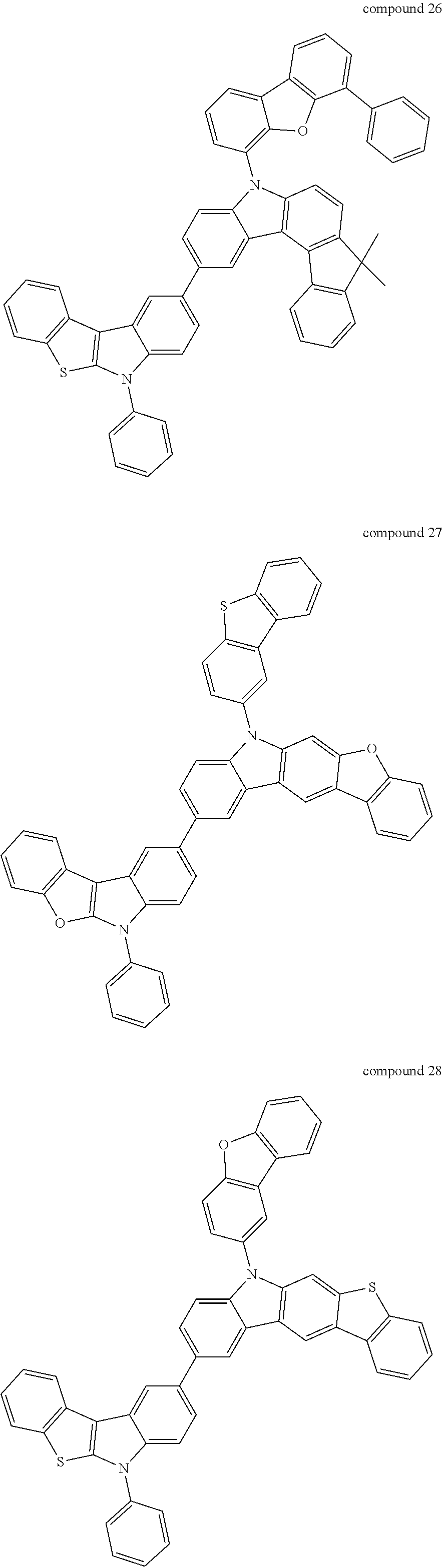 [SMILES: CC1(C)c2ccccc2-c2c1ccc1c2c2cc(-c3ccc4c(c3)c3c5ccccc5sc3n4-c3ccccc3)ccc2n1-c1cccc2c1oc1c(-c3ccccc3)cccc12 ; c1ccc(-n2c3ccc(-c4ccc5c(c4)c4cc6c(cc4n5-c4ccc5sc7ccccc7c5c4)oc4ccccc46)cc3c3c4ccccc4oc32)cc1 ; c1ccc(-n2c3ccc(-c4ccc5c(c4)c4cc6c(cc4n5-c4ccc5oc7ccccc7c5c4)sc4ccccc46)cc3c3c4ccccc4sc32)cc1]

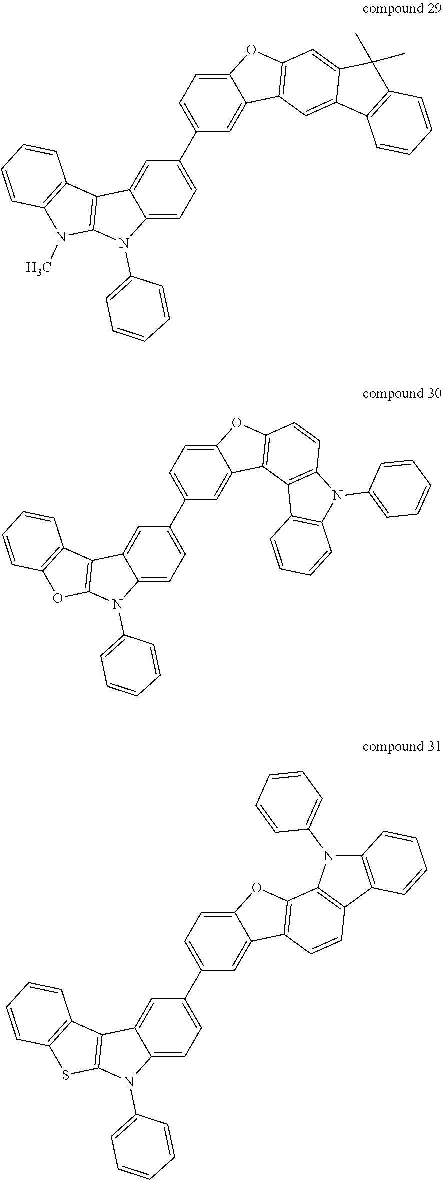 [SMILES: Cn1c2ccccc2c2c3cc(-c4ccc5oc6cc7c(cc6c5c4)-c4ccccc4C7(C)C)ccc3n(-c3ccccc3)c21 ; c1ccc(-n2c3ccccc3c3c4c(ccc32)oc2ccc(-c3ccc5c(c3)c3c6ccccc6oc3n5-c3ccccc3)cc24)cc1 ; c1ccc(-n2c3ccc(-c4ccc5oc6c(ccc7c8ccccc8n(-c8ccccc8)c76)c5c4)cc3c3c4ccccc4sc32)cc1]

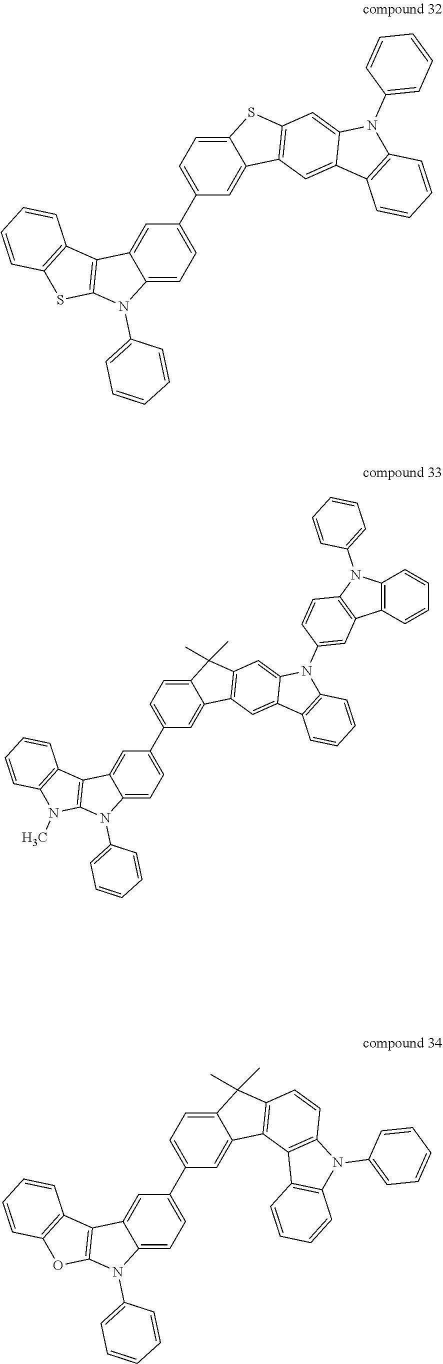 [SMILES: c1ccc(-n2c3ccccc3c3cc4c(cc32)sc2ccc(-c3ccc5c(c3)c3c6ccccc6sc3n5-c3ccccc3)cc24)cc1 ; Cn1c2ccccc2c2c3cc(-c4ccc5c(c4)-c4cc6c7ccccc7n(-c7ccc8c(c7)c7ccccc7n8-c7ccccc7)c6cc4C5(C)C)ccc3n(-c3ccccc3)c21 ; CC1(C)c2ccc(-c3ccc4c(c3)c3c5ccccc5oc3n4-c3ccccc3)cc2-c2c1ccc1c2c2ccccc2n1-c1ccccc1]

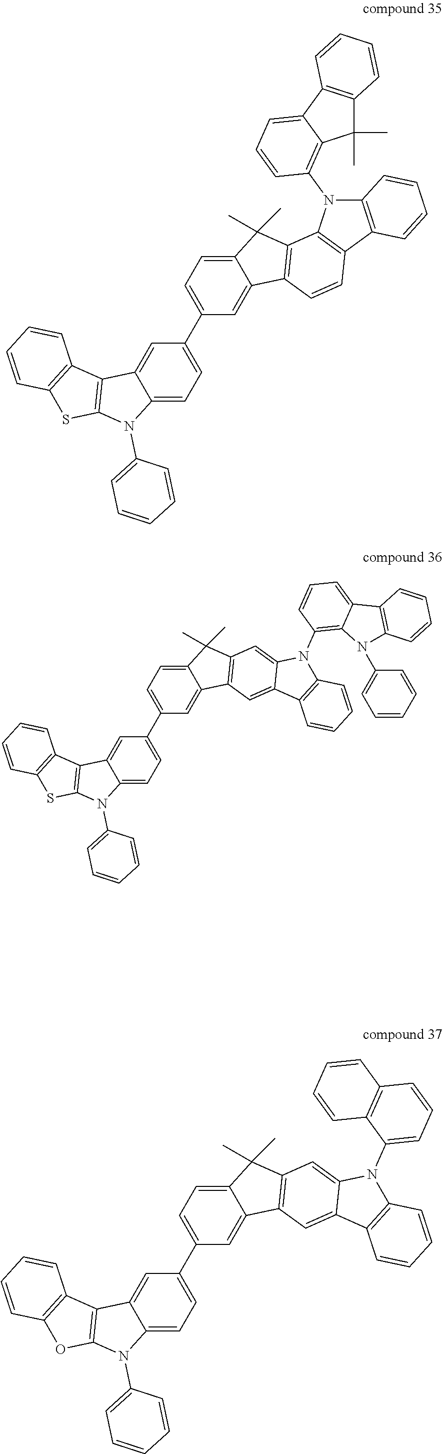 [SMILES: CC1(C)c2ccccc2-c2cccc(-n3c4ccccc4c4ccc5c(c43)C(C)(C)c3ccc(-c4ccc6c(c4)c4c7ccccc7sc4n6-c4ccccc4)cc3-5)c21 ; CC1(C)c2ccc(-c3ccc4c(c3)c3c5ccccc5sc3n4-c3ccccc3)cc2-c2cc3c4ccccc4n(-c4cccc5c6ccccc6n(-c6ccccc6)c45)c3cc21 ; CC1(C)c2ccc(-c3ccc4c(c3)c3c5ccccc5oc3n4-c3ccccc3)cc2-c2cc3c4ccccc4n(-c4cccc5ccccc45)c3cc21]

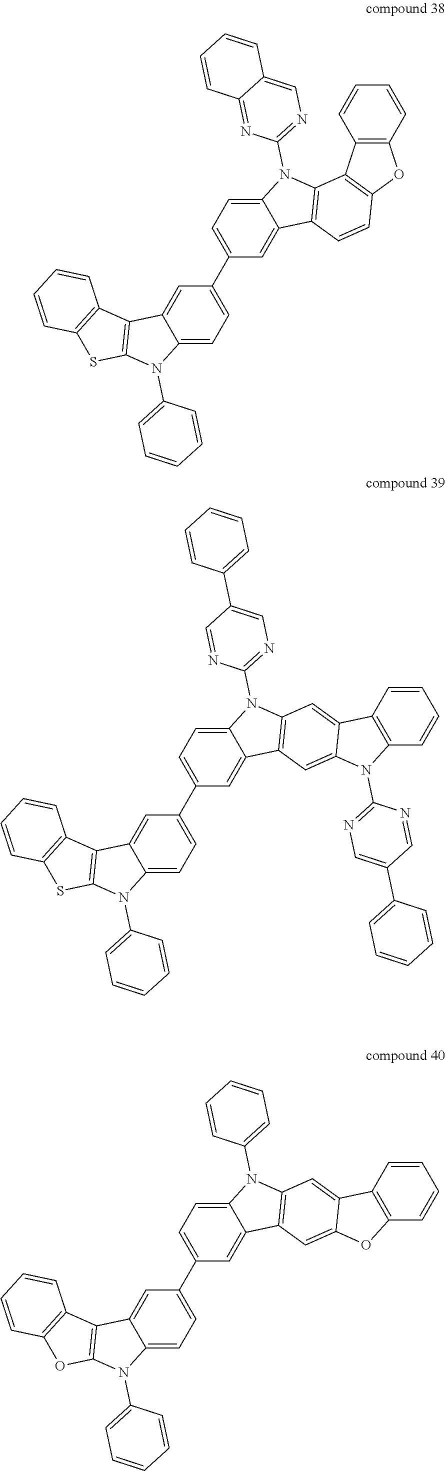 [SMILES: c1ccc(-n2c3ccc(-c4ccc5c(c4)c4ccc6oc7ccccc7c6c4n5-c4ncc5ccccc5n4)cc3c3c4ccccc4sc32)cc1 ; c1ccc(-c2cnc(-n3c4ccccc4c4cc5c(cc43)c3cc(-c4ccc6c(c4)c4c7ccccc7sc4n6-c4ccccc4)ccc3n5-c3ncc(-c4ccccc4)cn3)nc2)cc1 ; c1ccc(-n2c3ccc(-c4ccc5c(c4)c4c6ccccc6oc4n5-c4ccccc4)cc3c3cc4oc5ccccc5c4cc32)cc1]

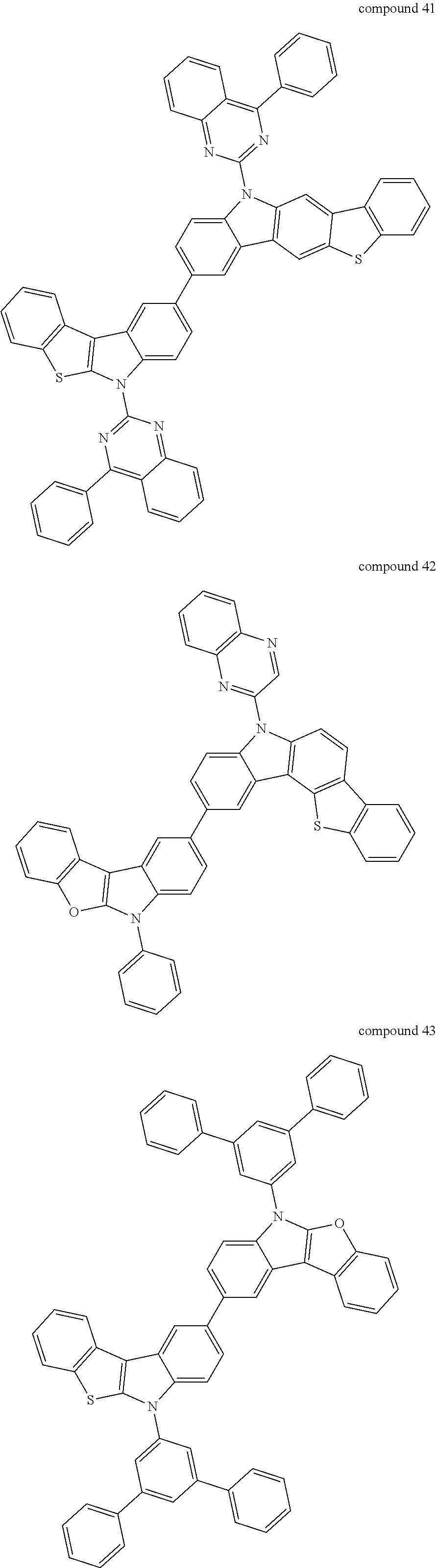 [SMILES: c1ccc(-c2nc(-n3c4ccc(-c5ccc6c(c5)c5c7ccccc7sc5n6-c5nc(-c6ccccc6)c6ccccc6n5)cc4c4cc5sc6ccccc6c5cc43)nc3ccccc23)cc1 ; c1ccc(-n2c3ccc(-c4ccc5c(c4)c4c6sc7ccccc7c6ccc4n5-c4cnc5ccccc5n4)cc3c3c4ccccc4oc32)cc1 ; c1ccc(-c2cc(-c3ccccc3)cc(-n3c4ccc(-c5ccc6c(c5)c5c7ccccc7sc5n6-c5cc(-c6ccccc6)cc(-c6ccccc6)c5)cc4c4c5ccccc5oc43)c2)cc1]

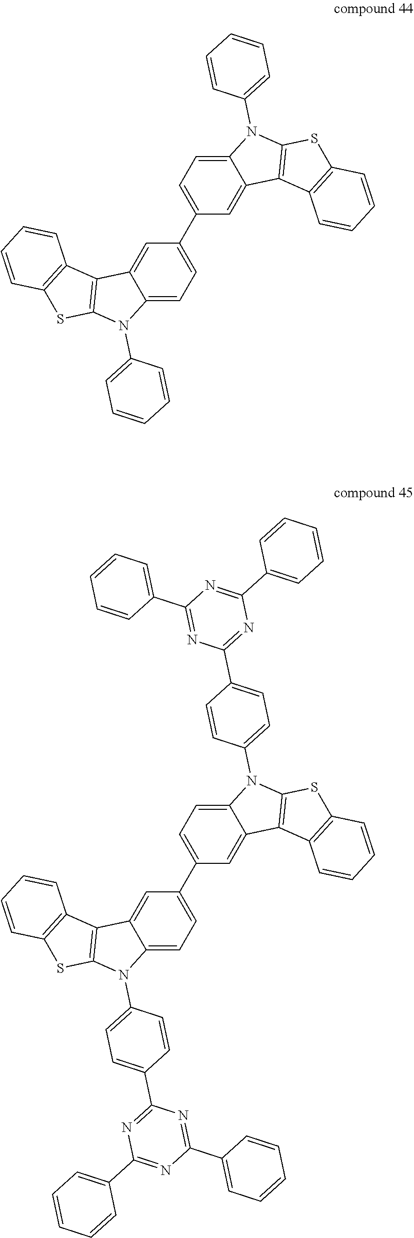 [SMILES: c1ccc(-n2c3ccc(-c4ccc5c(c4)c4c6ccccc6sc4n5-c4ccccc4)cc3c3c4ccccc4sc32)cc1 ; c1ccc(-c2nc(-c3ccccc3)nc(-c3ccc(-n4c5ccc(-c6ccc7c(c6)c6c8ccccc8sc6n7-c6ccc(-c7nc(-c8ccccc8)nc(-c8ccccc8)n7)cc6)cc5c5c6ccccc6sc54)cc3)n2)cc1]

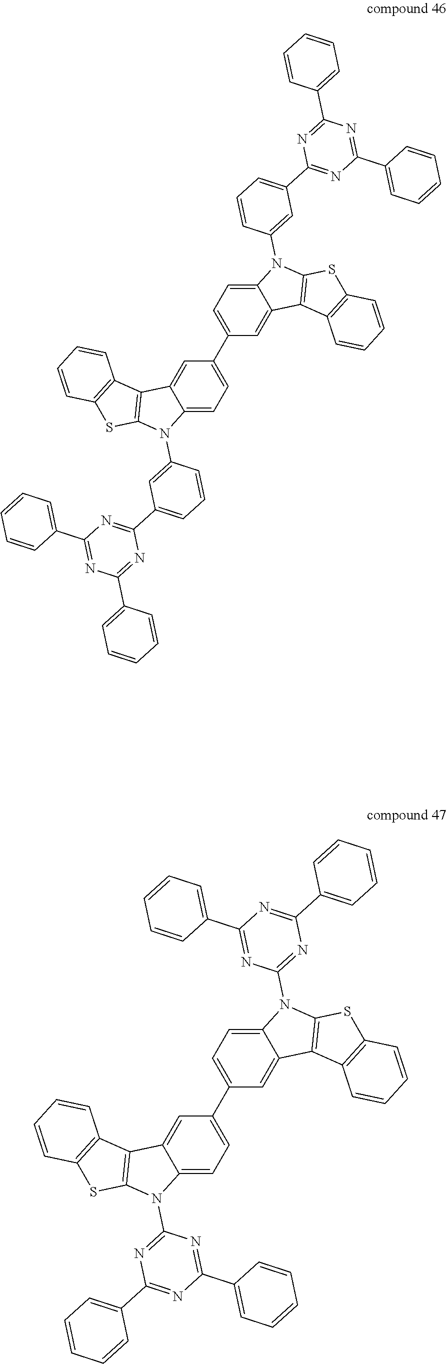 [SMILES: c1ccc(-c2nc(-c3ccccc3)nc(-c3cccc(-n4c5ccc(-c6ccc7c(c6)c6c8ccccc8sc6n7-c6cccc(-c7nc(-c8ccccc8)nc(-c8ccccc8)n7)c6)cc5c5c6ccccc6sc54)c3)n2)cc1 ; c1ccc(-c2nc(-c3ccccc3)nc(-n3c4ccc(-c5ccc6c(c5)c5c7ccccc7sc5n6-c5nc(-c6ccccc6)nc(-c6ccccc6)n5)cc4c4c5ccccc5sc43)n2)cc1]

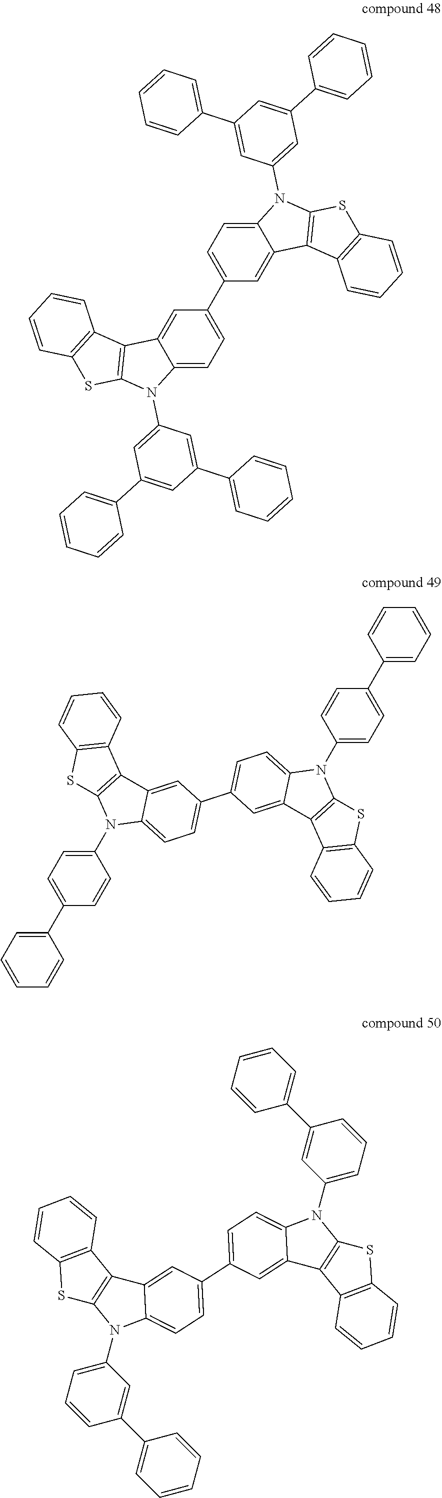 [SMILES: c1ccc(-c2cc(-c3ccccc3)cc(-n3c4ccc(-c5ccc6c(c5)c5c7ccccc7sc5n6-c5cc(-c6ccccc6)cc(-c6ccccc6)c5)cc4c4c5ccccc5sc43)c2)cc1 ; c1ccc(-c2ccc(-n3c4ccc(-c5ccc6c(c5)c5c7ccccc7sc5n6-c5ccc(-c6ccccc6)cc5)cc4c4c5ccccc5sc43)cc2)cc1 ; c1ccc(-c2cccc(-n3c4ccc(-c5ccc6c(c5)c5c7ccccc7sc5n6-c5cccc(-c6ccccc6)c5)cc4c4c5ccccc5sc43)c2)cc1]

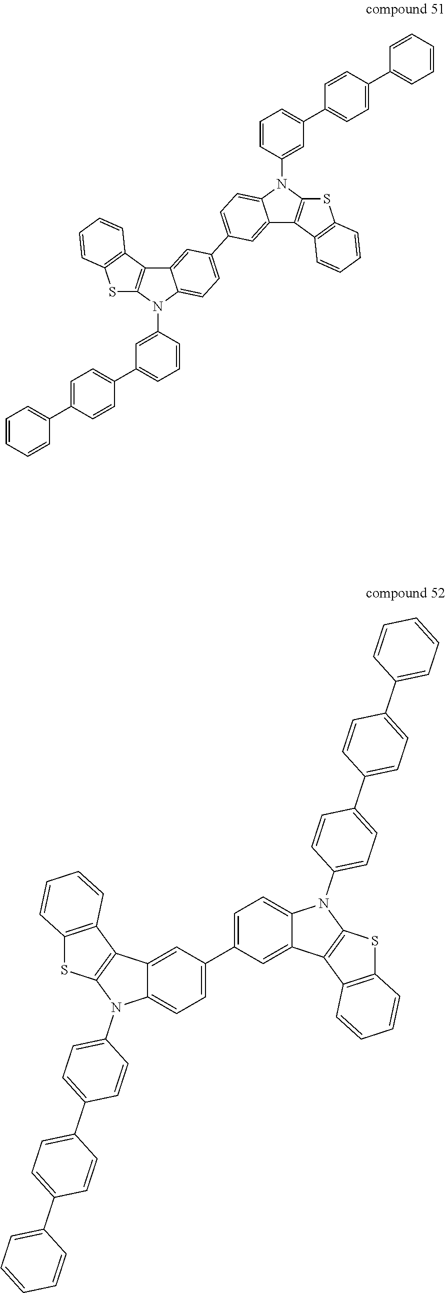 [SMILES: c1ccc(-c2ccc(-c3cccc(-n4c5ccc(-c6ccc7c(c6)c6c8ccccc8sc6n7-c6cccc(-c7ccc(-c8ccccc8)cc7)c6)cc5c5c6ccccc6sc54)c3)cc2)cc1 ; c1ccc(-c2ccc(-c3ccc(-n4c5ccc(-c6ccc7c(c6)c6c8ccccc8sc6n7-c6ccc(-c7ccc(-c8ccccc8)cc7)cc6)cc5c5c6ccccc6sc54)cc3)cc2)cc1]

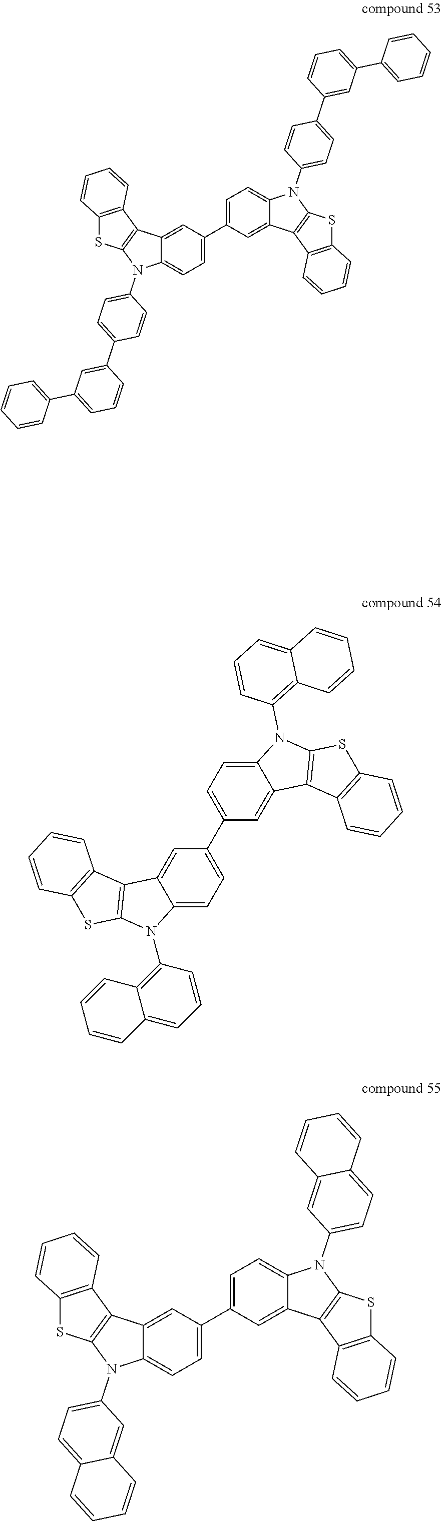 [SMILES: c1ccc(-c2cccc(-c3ccc(-n4c5ccc(-c6ccc7c(c6)c6c8ccccc8sc6n7-c6ccc(-c7cccc(-c8ccccc8)c7)cc6)cc5c5c6ccccc6sc54)cc3)c2)cc1 ; c1ccc2c(-n3c4ccc(-c5ccc6c(c5)c5c7ccccc7sc5n6-c5cccc6ccccc56)cc4c4c5ccccc5sc43)cccc2c1 ; c1ccc2cc(-n3c4ccc(-c5ccc6c(c5)c5c7ccccc7sc5n6-c5ccc6ccccc6c5)cc4c4c5ccccc5sc43)ccc2c1]

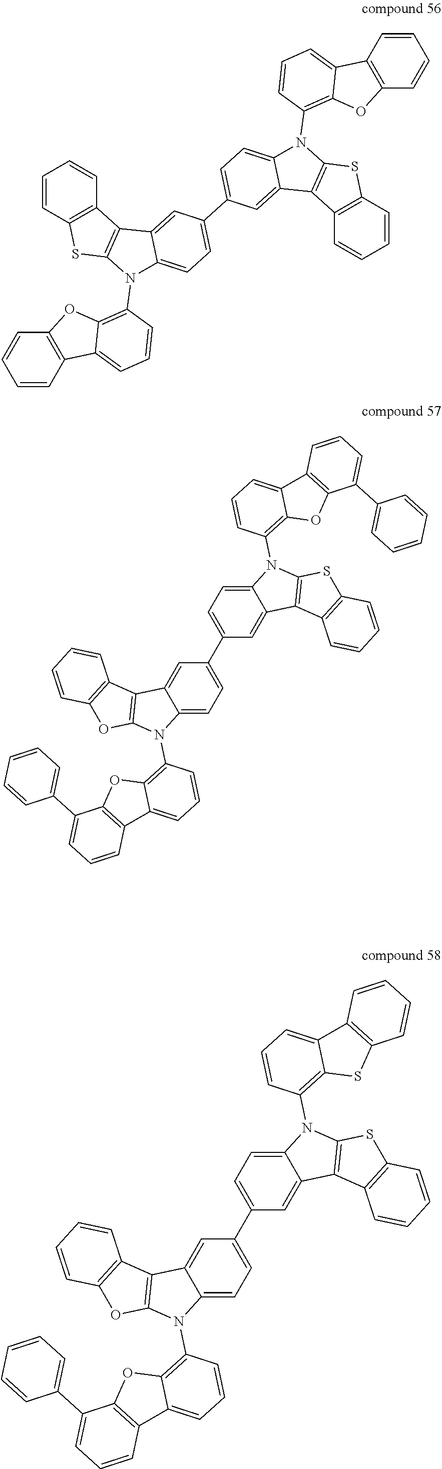 [SMILES: c1ccc2c(c1)oc1c(-n3c4ccc(-c5ccc6c(c5)c5c7ccccc7sc5n6-c5cccc6c5oc5ccccc56)cc4c4c5ccccc5sc43)cccc12 ; c1ccc(-c2cccc3c2oc2c(-n4c5ccc(-c6ccc7c(c6)c6c8ccccc8sc6n7-c6cccc7c6oc6c(-c8ccccc8)cccc67)cc5c5c6ccccc6oc54)cccc23)cc1 ; c1ccc(-c2cccc3c2oc2c(-n4c5ccc(-c6ccc7c(c6)c6c8ccccc8sc6n7-c6cccc7c6sc6ccccc67)cc5c5c6ccccc6oc54)cccc23)cc1]

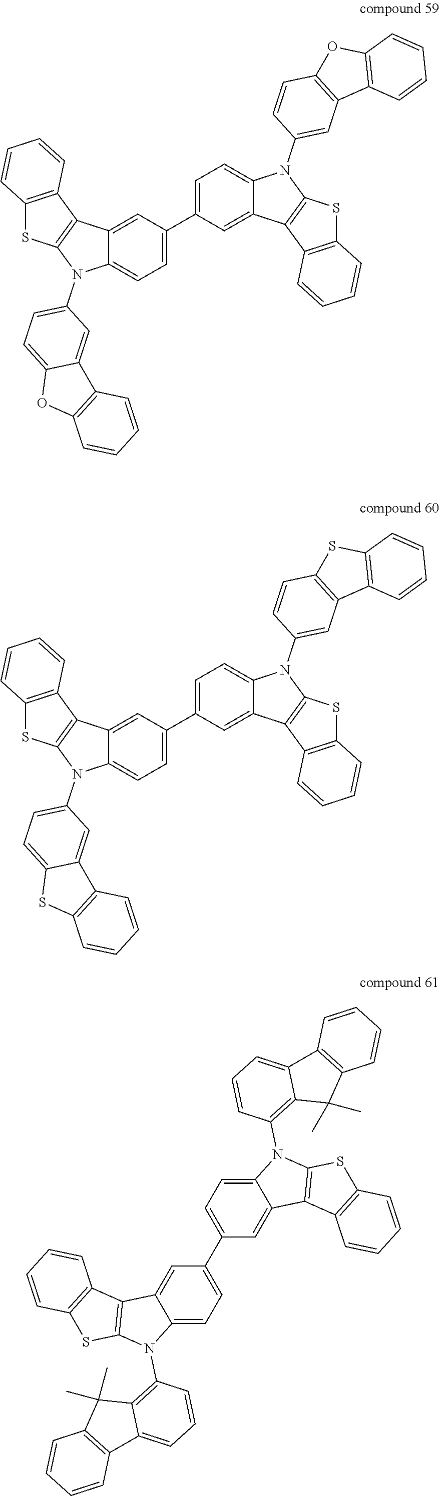 [SMILES: c1ccc2c(c1)oc1ccc(-n3c4ccc(-c5ccc6c(c5)c5c7ccccc7sc5n6-c5ccc6oc7ccccc7c6c5)cc4c4c5ccccc5sc43)cc12 ; c1ccc2c(c1)sc1ccc(-n3c4ccc(-c5ccc6c(c5)c5c7ccccc7sc5n6-c5ccc6sc7ccccc7c6c5)cc4c4c5ccccc5sc43)cc12 ; CC1(C)c2ccccc2-c2cccc(-n3c4ccc(-c5ccc6c(c5)c5c7ccccc7sc5n6-c5cccc6c5C(C)(C)c5ccccc5-6)cc4c4c5ccccc5sc43)c21]

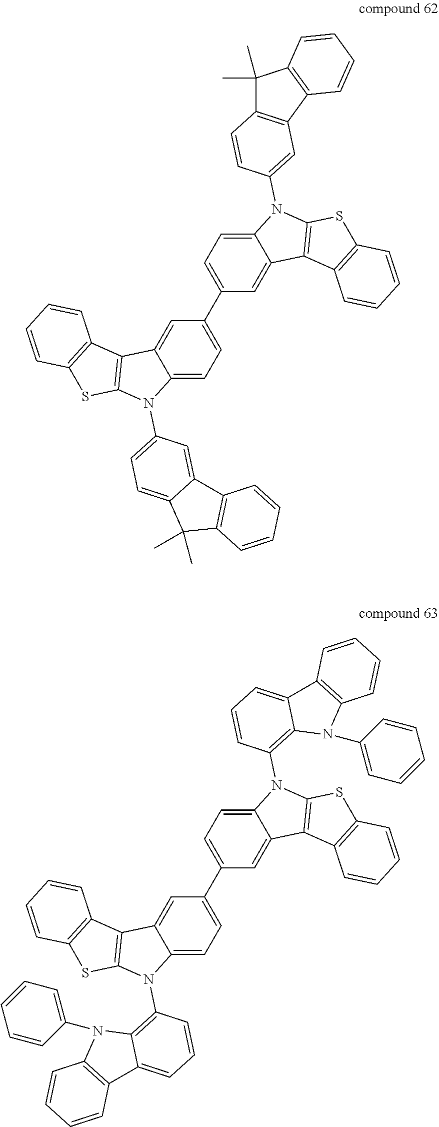 [SMILES: CC1(C)c2ccccc2-c2cc(-n3c4ccc(-c5ccc6c(c5)c5c7ccccc7sc5n6-c5ccc6c(c5)-c5ccccc5C6(C)C)cc4c4c5ccccc5sc43)ccc21 ; c1ccc(-n2c3ccccc3c3cccc(-n4c5ccc(-c6ccc7c(c6)c6c8ccccc8sc6n7-c6cccc7c8ccccc8n(-c8ccccc8)c67)cc5c5c6ccccc6sc54)c32)cc1]

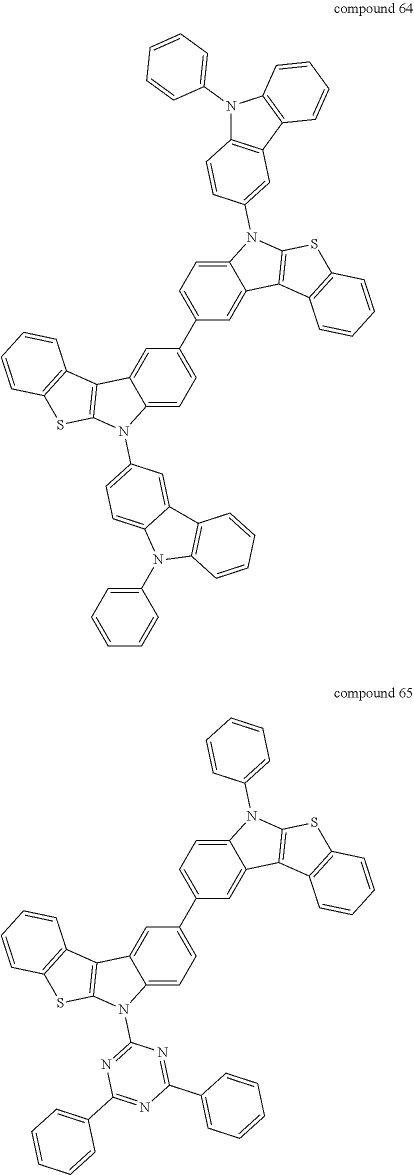 [SMILES: c1ccc(-n2c3ccccc3c3cc(-n4c5ccc(-c6ccc7c(c6)c6c8ccccc8sc6n7-c6ccc7c(c6)c6ccccc6n7-c6ccccc6)cc5c5c6ccccc6sc54)ccc32)cc1 ; c1ccc(-c2nc(-c3ccccc3)nc(-n3c4ccc(-c5ccc6c(c5)c5c7ccccc7sc5n6-c5ccccc5)cc4c4c5ccccc5sc43)n2)cc1]

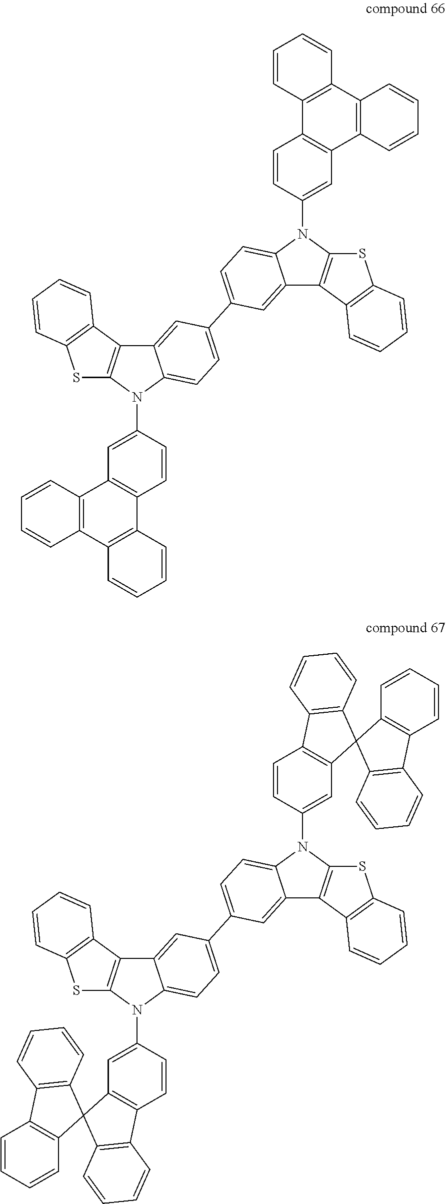 [SMILES: c1ccc2c(c1)sc1c2c2cc(-c3ccc4c(c3)c3c5ccccc5sc3n4-c3ccc4c5ccccc5c5ccccc5c4c3)ccc2n1-c1ccc2c3ccccc3c3ccccc3c2c1 ; c1ccc2c(c1)-c1ccccc1C21c2ccccc2-c2ccc(-n3c4ccc(-c5ccc6c(c5)c5c7ccccc7sc5n6-c5ccc6c(c5)C5(c7ccccc7-c7ccccc75)c5ccccc5-6)cc4c4c5ccccc5sc43)cc21]

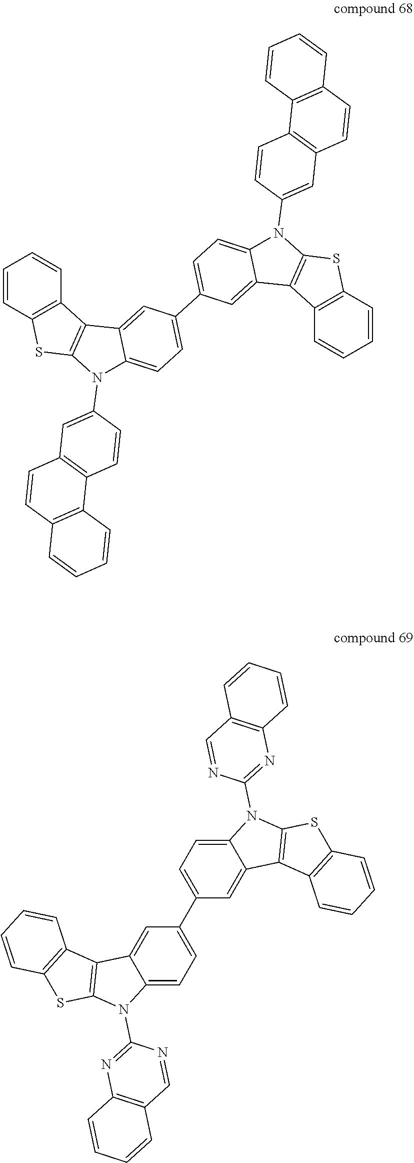 [SMILES: c1ccc2c(c1)ccc1cc(-n3c4ccc(-c5ccc6c(c5)c5c7ccccc7sc5n6-c5ccc6c(ccc7ccccc76)c5)cc4c4c5ccccc5sc43)ccc12 ; c1ccc2nc(-n3c4ccc(-c5ccc6c(c5)c5c7ccccc7sc5n6-c5ncc6ccccc6n5)cc4c4c5ccccc5sc43)ncc2c1]

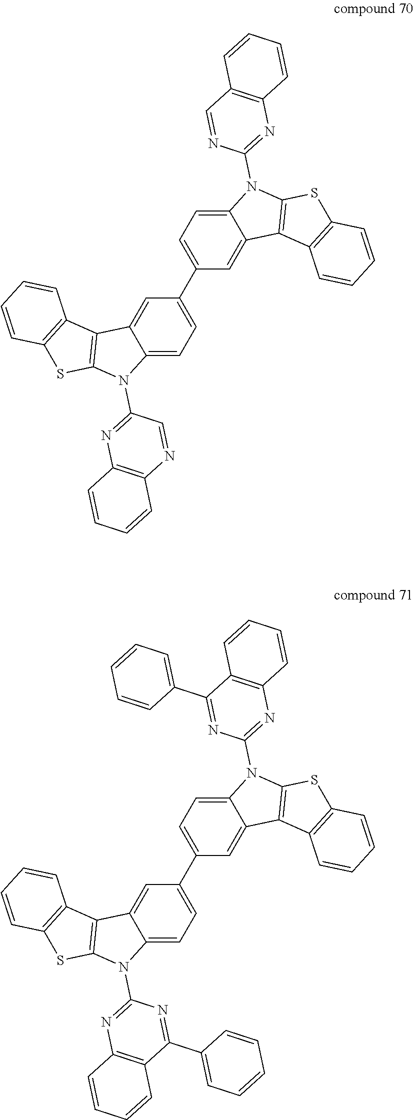 [SMILES: c1ccc2nc(-n3c4ccc(-c5ccc6c(c5)c5c7ccccc7sc5n6-c5cnc6ccccc6n5)cc4c4c5ccccc5sc43)ncc2c1 ; c1ccc(-c2nc(-n3c4ccc(-c5ccc6c(c5)c5c7ccccc7sc5n6-c5nc(-c6ccccc6)c6ccccc6n5)cc4c4c5ccccc5sc43)nc3ccccc23)cc1]

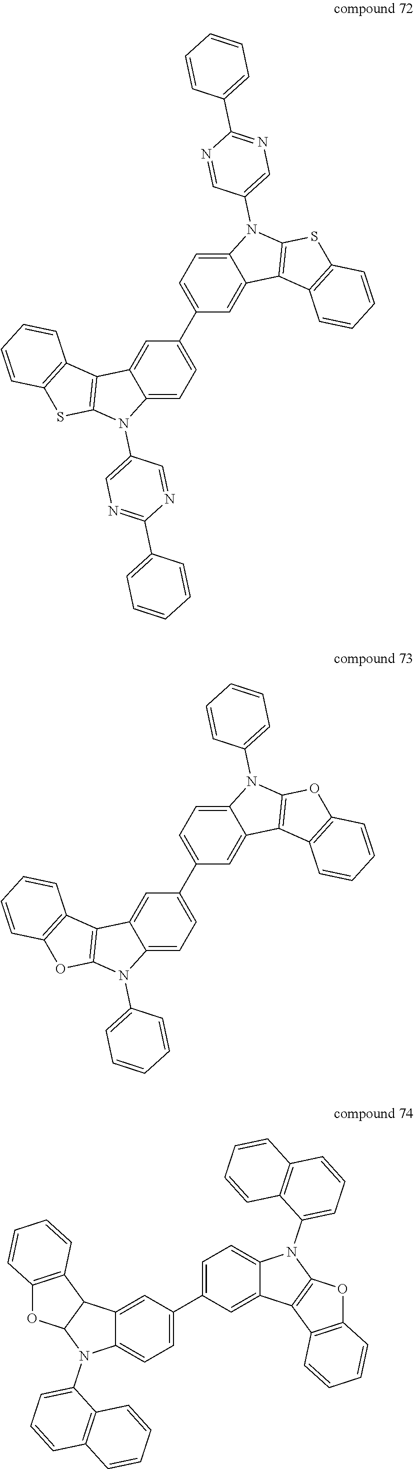 [SMILES: c1ccc(-c2ncc(-n3c4ccc(-c5ccc6c(c5)c5c7ccccc7sc5n6-c5cnc(-c6ccccc6)nc5)cc4c4c5ccccc5sc43)cn2)cc1 ; c1ccc(-n2c3ccc(-c4ccc5c(c4)c4c6ccccc6oc4n5-c4ccccc4)cc3c3c4ccccc4oc32)cc1 ; c1ccc2c(c1)OC1C2c2cc(-c3ccc4c(c3)c3c5ccccc5oc3n4-c3cccc4ccccc34)ccc2N1c1cccc2ccccc12]

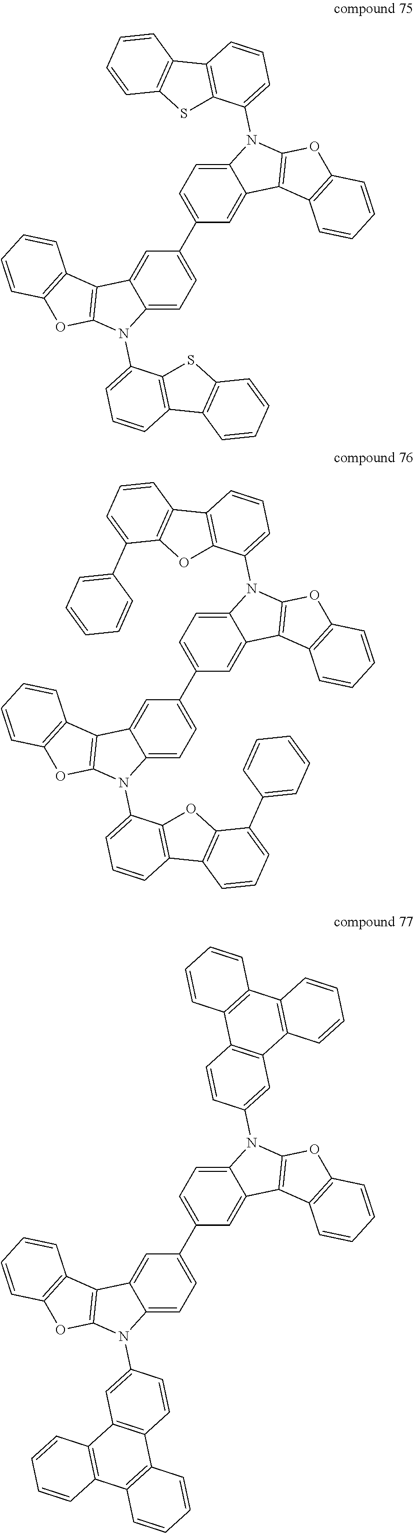 [SMILES: c1ccc2c(c1)oc1c2c2cc(-c3ccc4c(c3)c3c5ccccc5oc3n4-c3cccc4c3sc3ccccc34)ccc2n1-c1cccc2c1sc1ccccc12 ; c1ccc(-c2cccc3c2oc2c(-n4c5ccc(-c6ccc7c(c6)c6c8ccccc8oc6n7-c6cccc7c6oc6c(-c8ccccc8)cccc67)cc5c5c6ccccc6oc54)cccc23)cc1 ; c1ccc2c(c1)oc1c2c2cc(-c3ccc4c(c3)c3c5ccccc5oc3n4-c3ccc4c5ccccc5c5ccccc5c4c3)ccc2n1-c1ccc2c3ccccc3c3ccccc3c2c1]

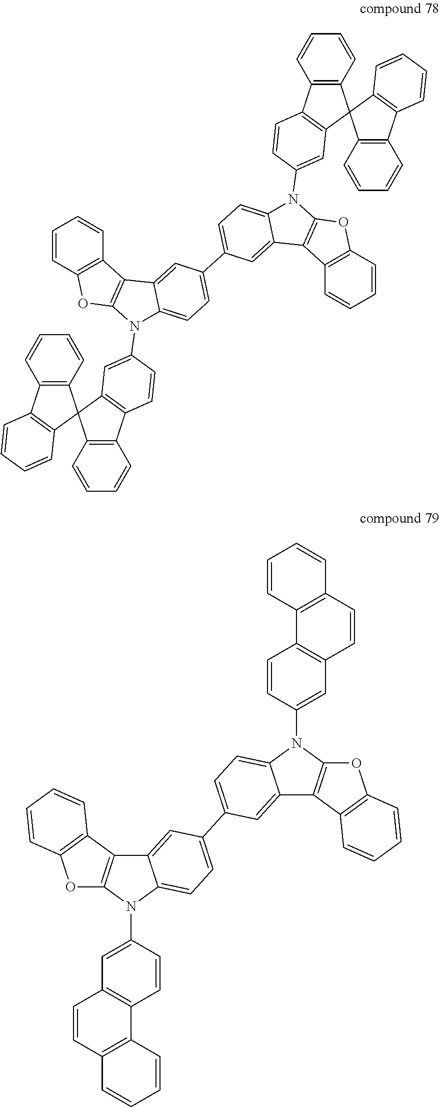 [SMILES: c1ccc2c(c1)-c1ccccc1C21c2ccccc2-c2ccc(-n3c4ccc(-c5ccc6c(c5)c5c7ccccc7oc5n6-c5ccc6c(c5)C5(c7ccccc7-c7ccccc75)c5ccccc5-6)cc4c4c5ccccc5oc43)cc21 ; c1ccc2c(c1)ccc1cc(-n3c4ccc(-c5ccc6c(c5)c5c7ccccc7oc5n6-c5ccc6c(ccc7ccccc76)c5)cc4c4c5ccccc5oc43)ccc12]

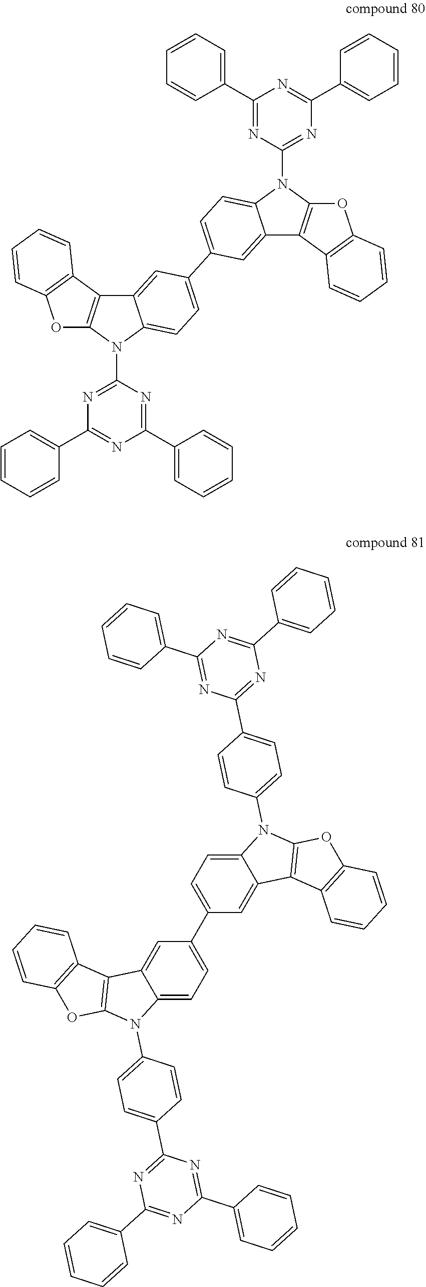 [SMILES: c1ccc(-c2nc(-c3ccccc3)nc(-n3c4ccc(-c5ccc6c(c5)c5c7ccccc7oc5n6-c5nc(-c6ccccc6)nc(-c6ccccc6)n5)cc4c4c5ccccc5oc43)n2)cc1 ; c1ccc(-c2nc(-c3ccccc3)nc(-c3ccc(-n4c5ccc(-c6ccc7c(c6)c6c8ccccc8oc6n7-c6ccc(-c7nc(-c8ccccc8)nc(-c8ccccc8)n7)cc6)cc5c5c6ccccc6oc54)cc3)n2)cc1]

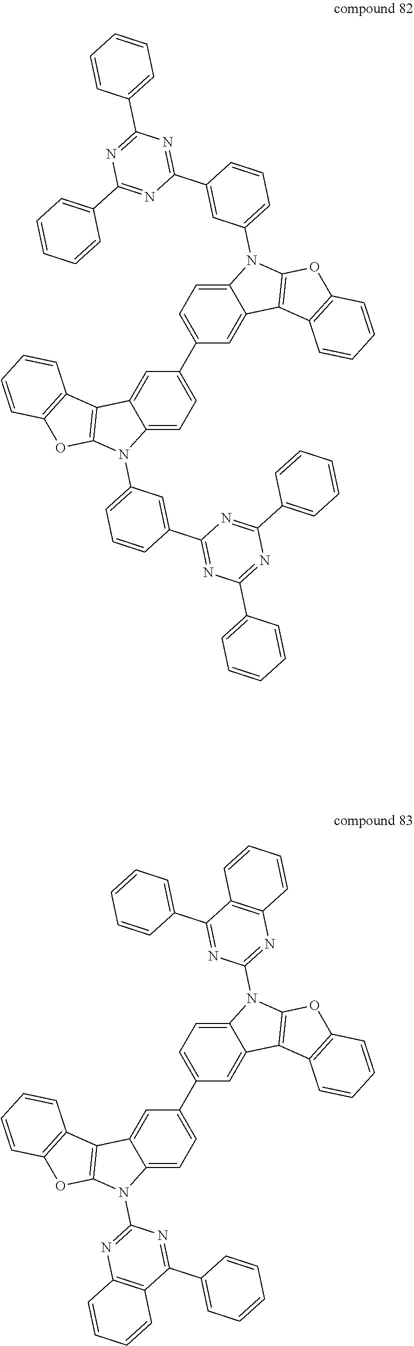 [SMILES: c1ccc(-c2nc(-c3ccccc3)nc(-c3cccc(-n4c5ccc(-c6ccc7c(c6)c6c8ccccc8oc6n7-c6cccc(-c7nc(-c8ccccc8)nc(-c8ccccc8)n7)c6)cc5c5c6ccccc6oc54)c3)n2)cc1 ; c1ccc(-c2nc(-n3c4ccc(-c5ccc6c(c5)c5c7ccccc7oc5n6-c5nc(-c6ccccc6)c6ccccc6n5)cc4c4c5ccccc5oc43)nc3ccccc23)cc1]

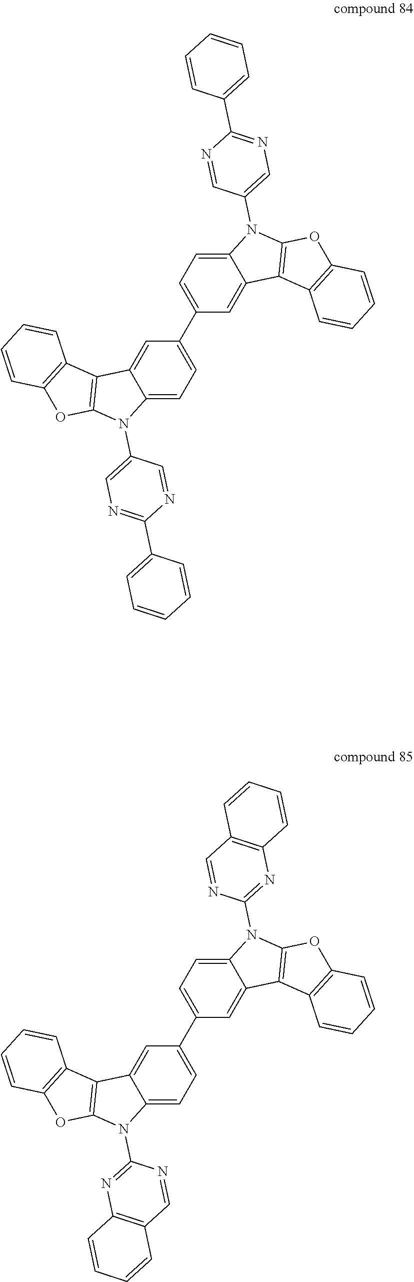 [SMILES: c1ccc(-c2ncc(-n3c4ccc(-c5ccc6c(c5)c5c7ccccc7oc5n6-c5cnc(-c6ccccc6)nc5)cc4c4c5ccccc5oc43)cn2)cc1 ; c1ccc2nc(-n3c4ccc(-c5ccc6c(c5)c5c7ccccc7oc5n6-c5ncc6ccccc6n5)cc4c4c5ccccc5oc43)ncc2c1]

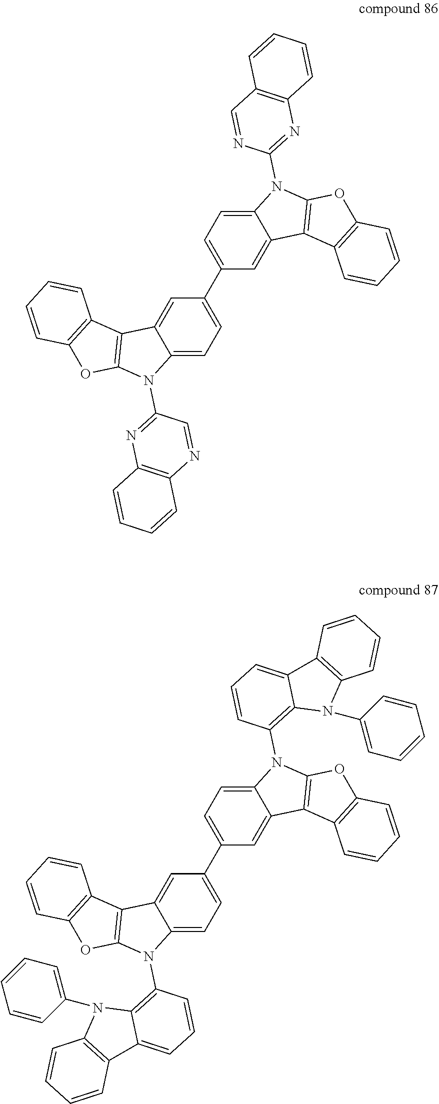 [SMILES: c1ccc2nc(-n3c4ccc(-c5ccc6c(c5)c5c7ccccc7oc5n6-c5cnc6ccccc6n5)cc4c4c5ccccc5oc43)ncc2c1 ; c1ccc(-n2c3ccccc3c3cccc(-n4c5ccc(-c6ccc7c(c6)c6c8ccccc8oc6n7-c6cccc7c8ccccc8n(-c8ccccc8)c67)cc5c5c6ccccc6oc54)c32)cc1]

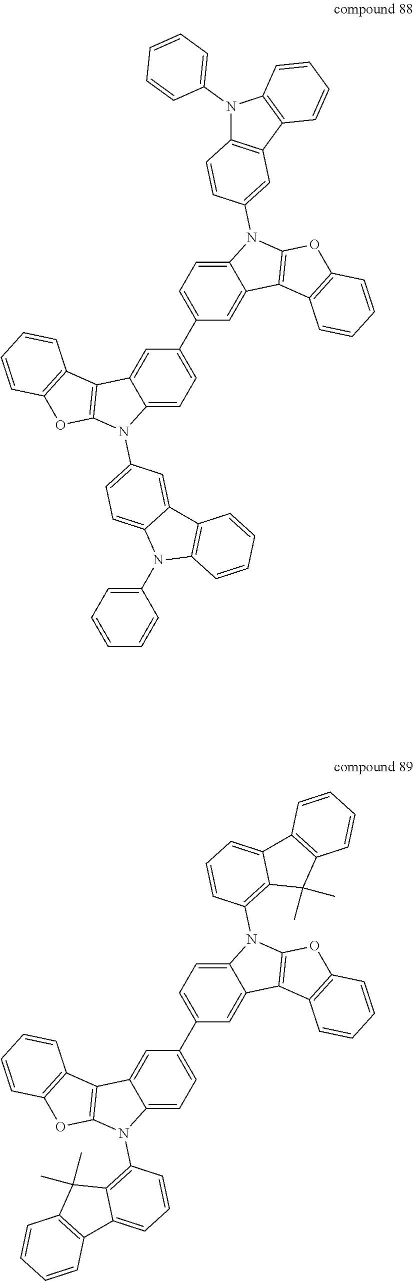 [SMILES: c1ccc(-n2c3ccccc3c3cc(-n4c5ccc(-c6ccc7c(c6)c6c8ccccc8oc6n7-c6ccc7c(c6)c6ccccc6n7-c6ccccc6)cc5c5c6ccccc6oc54)ccc32)cc1 ; CC1(C)c2ccccc2-c2cccc(-n3c4ccc(-c5ccc6c(c5)c5c7ccccc7oc5n6-c5cccc6c5C(C)(C)c5ccccc5-6)cc4c4c5ccccc5oc43)c21]

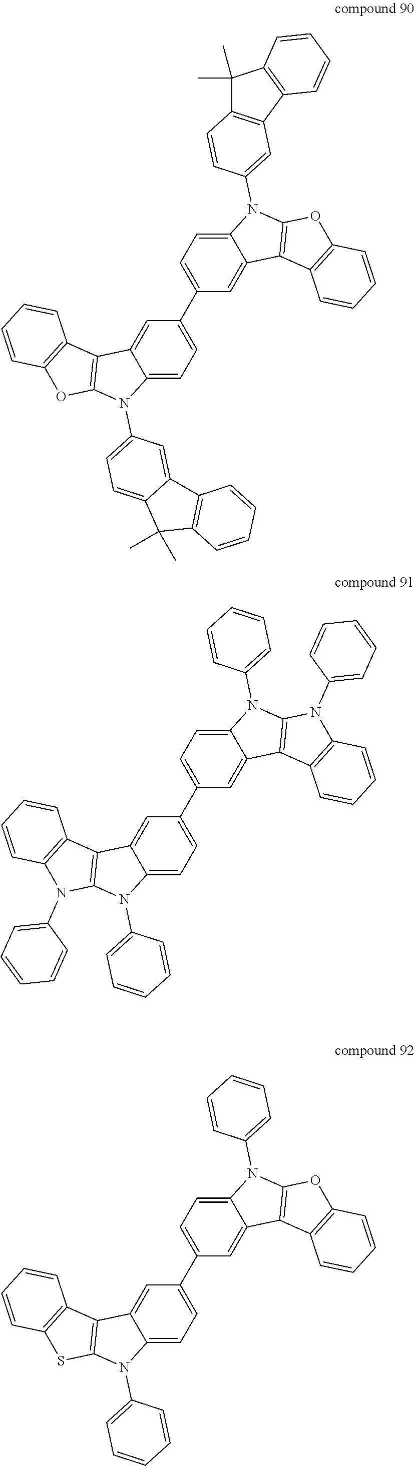 [SMILES: CC1(C)c2ccccc2-c2cc(-n3c4ccc(-c5ccc6c(c5)c5c7ccccc7oc5n6-c5ccc6c(c5)-c5ccccc5C6(C)C)cc4c4c5ccccc5oc43)ccc21 ; c1ccc(-n2c3ccccc3c3c4cc(-c5ccc6c(c5)c5c7ccccc7n(-c7ccccc7)c5n6-c5ccccc5)ccc4n(-c4ccccc4)c32)cc1 ; c1ccc(-n2c3ccc(-c4ccc5c(c4)c4c6ccccc6sc4n5-c4ccccc4)cc3c3c4ccccc4oc32)cc1]

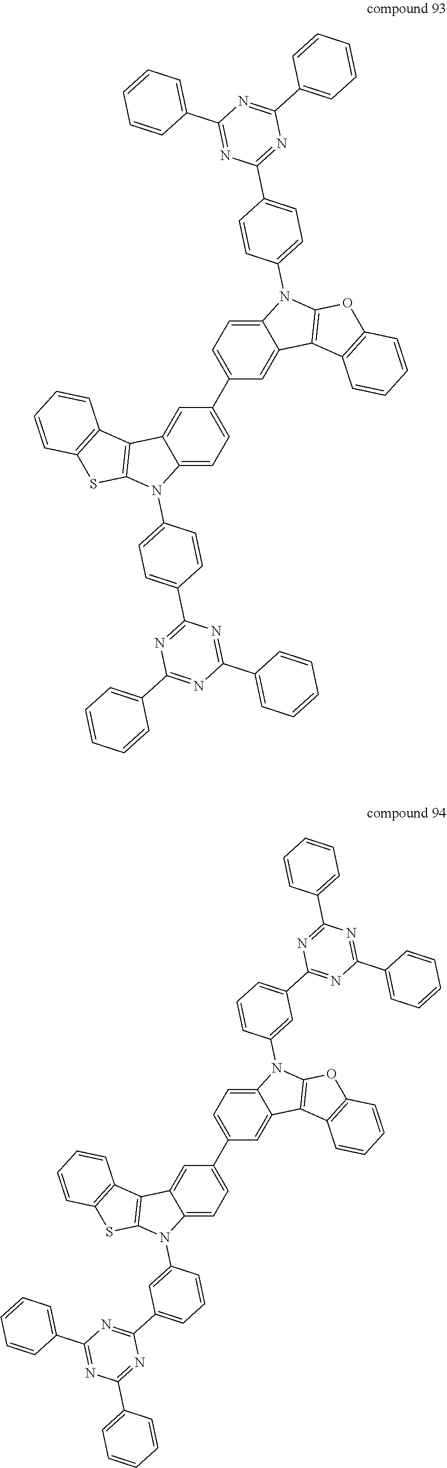 [SMILES: c1ccc(-c2nc(-c3ccccc3)nc(-c3ccc(-n4c5ccc(-c6ccc7c(c6)c6c8ccccc8sc6n7-c6ccc(-c7nc(-c8ccccc8)nc(-c8ccccc8)n7)cc6)cc5c5c6ccccc6oc54)cc3)n2)cc1 ; c1ccc(-c2nc(-c3ccccc3)nc(-c3cccc(-n4c5ccc(-c6ccc7c(c6)c6c8ccccc8sc6n7-c6cccc(-c7nc(-c8ccccc8)nc(-c8ccccc8)n7)c6)cc5c5c6ccccc6oc54)c3)n2)cc1]

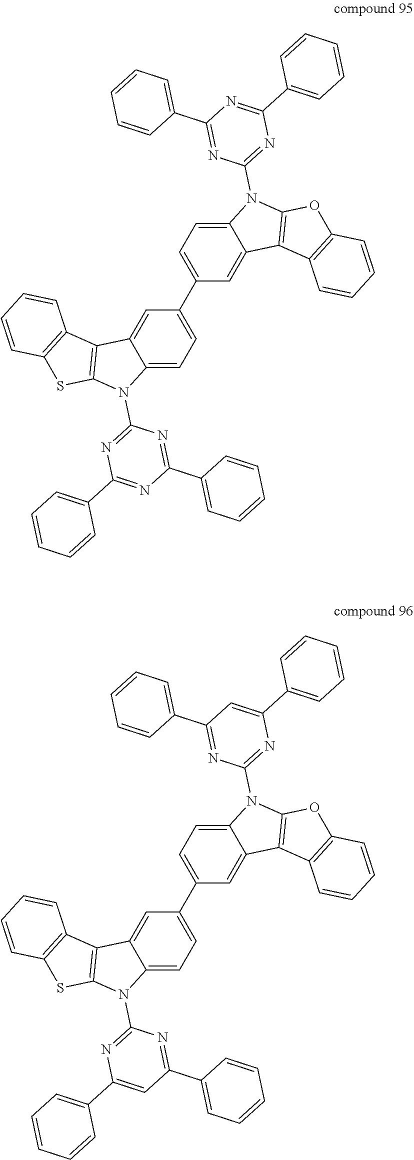 [SMILES: c1ccc(-c2nc(-c3ccccc3)nc(-n3c4ccc(-c5ccc6c(c5)c5c7ccccc7sc5n6-c5nc(-c6ccccc6)nc(-c6ccccc6)n5)cc4c4c5ccccc5oc43)n2)cc1 ; c1ccc(-c2cc(-c3ccccc3)nc(-n3c4ccc(-c5ccc6c(c5)c5c7ccccc7sc5n6-c5nc(-c6ccccc6)cc(-c6ccccc6)n5)cc4c4c5ccccc5oc43)n2)cc1]

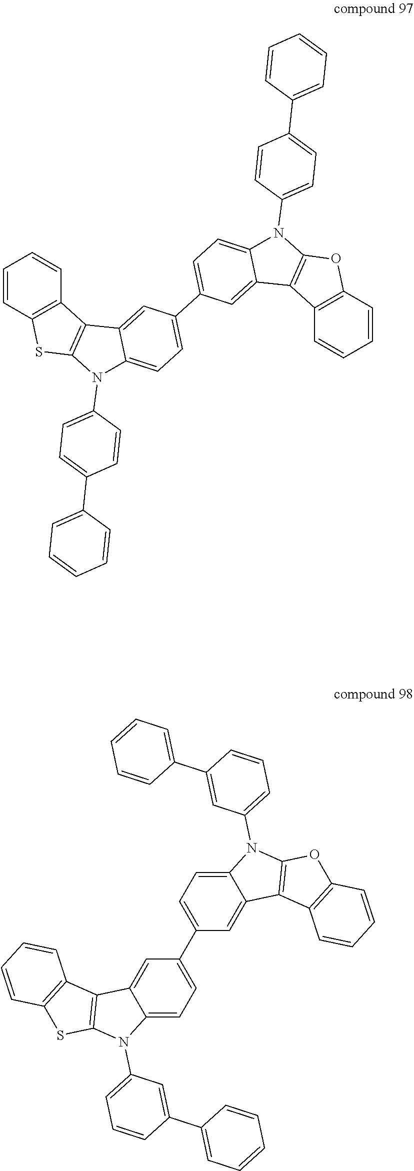 [SMILES: c1ccc(-c2ccc(-n3c4ccc(-c5ccc6c(c5)c5c7ccccc7sc5n6-c5ccc(-c6ccccc6)cc5)cc4c4c5ccccc5oc43)cc2)cc1 ; c1ccc(-c2cccc(-n3c4ccc(-c5ccc6c(c5)c5c7ccccc7sc5n6-c5cccc(-c6ccccc6)c5)cc4c4c5ccccc5oc43)c2)cc1]

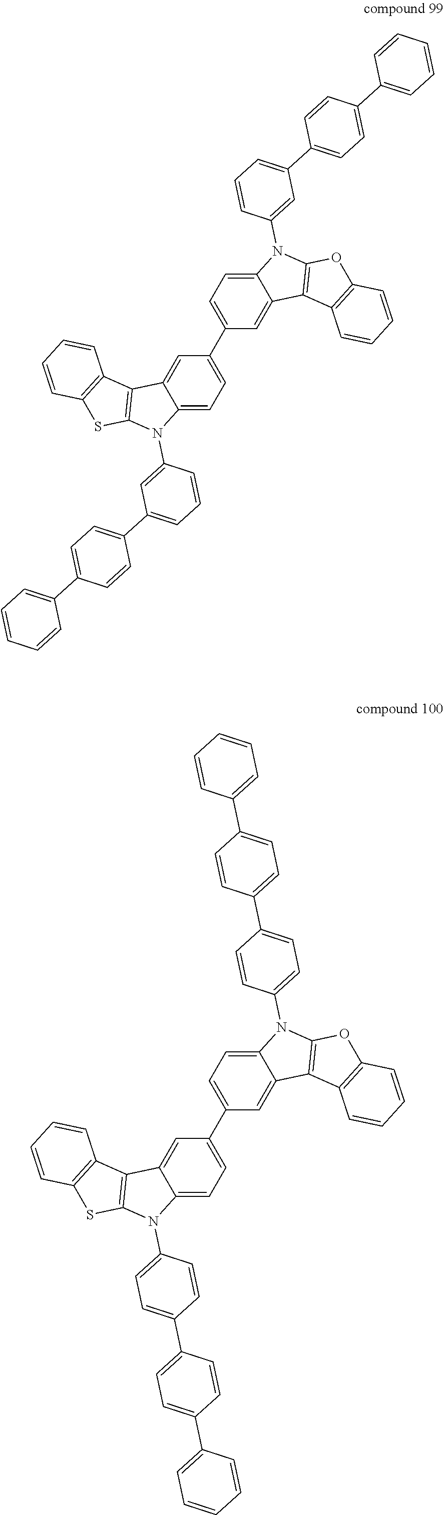 [SMILES: c1ccc(-c2ccc(-c3cccc(-n4c5ccc(-c6ccc7c(c6)c6c8ccccc8sc6n7-c6cccc(-c7ccc(-c8ccccc8)cc7)c6)cc5c5c6ccccc6oc54)c3)cc2)cc1 ; c1ccc(-c2ccc(-c3ccc(-n4c5ccc(-c6ccc7c(c6)c6c8ccccc8sc6n7-c6ccc(-c7ccc(-c8ccccc8)cc7)cc6)cc5c5c6ccccc6oc54)cc3)cc2)cc1]

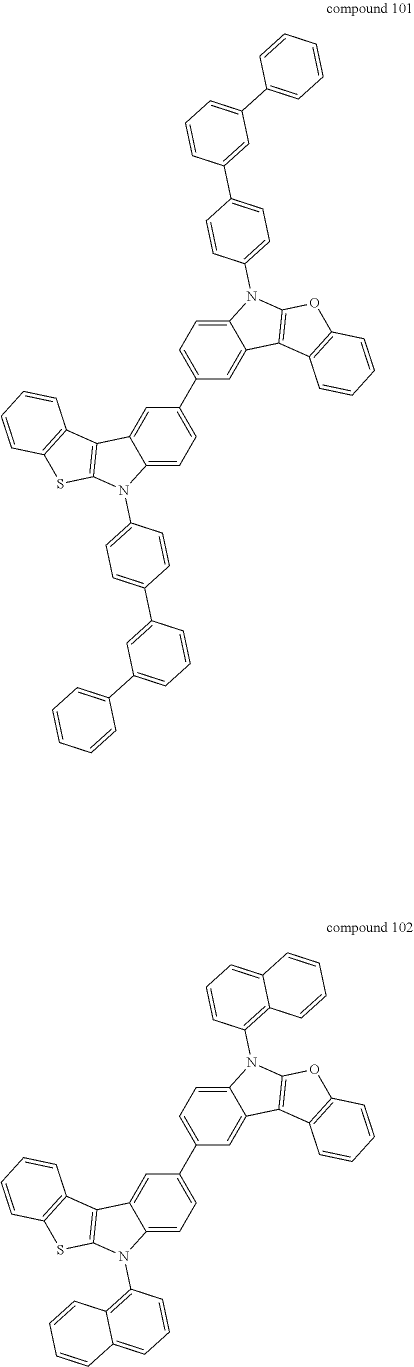 [SMILES: c1ccc(-c2cccc(-c3ccc(-n4c5ccc(-c6ccc7c(c6)c6c8ccccc8sc6n7-c6ccc(-c7cccc(-c8ccccc8)c7)cc6)cc5c5c6ccccc6oc54)cc3)c2)cc1 ; c1ccc2c(-n3c4ccc(-c5ccc6c(c5)c5c7ccccc7sc5n6-c5cccc6ccccc56)cc4c4c5ccccc5oc43)cccc2c1]

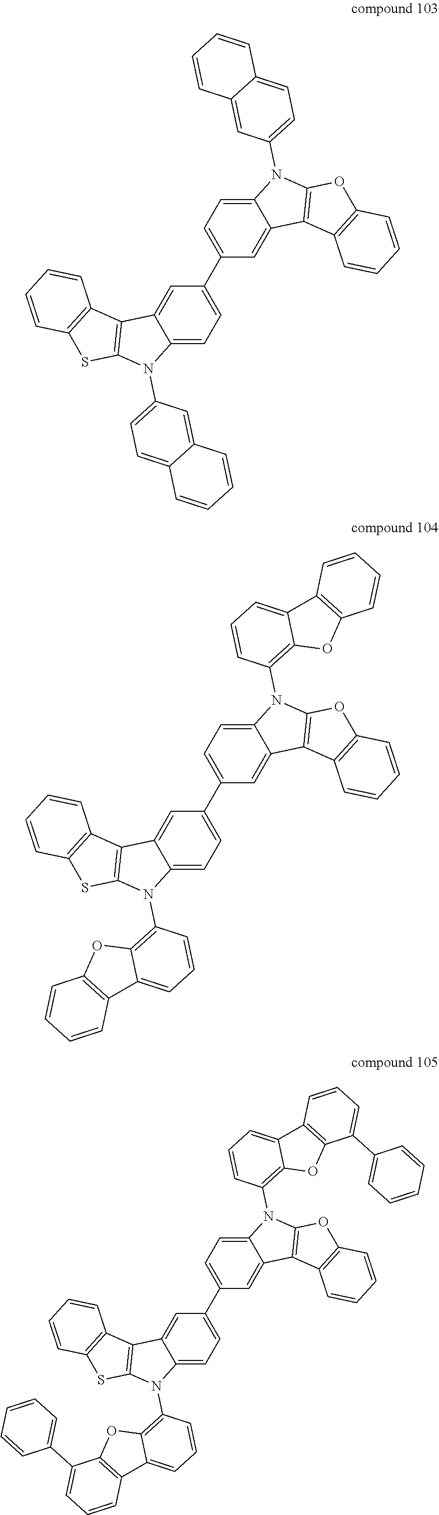 [SMILES: c1ccc2cc(-n3c4ccc(-c5ccc6c(c5)c5c7ccccc7sc5n6-c5ccc6ccccc6c5)cc4c4c5ccccc5oc43)ccc2c1 ; c1ccc2c(c1)oc1c(-n3c4ccc(-c5ccc6c(c5)c5c7ccccc7sc5n6-c5cccc6c5oc5ccccc56)cc4c4c5ccccc5oc43)cccc12 ; c1ccc(-c2cccc3c2oc2c(-n4c5ccc(-c6ccc7c(c6)c6c8ccccc8sc6n7-c6cccc7c6oc6c(-c8ccccc8)cccc67)cc5c5c6ccccc6oc54)cccc23)cc1]

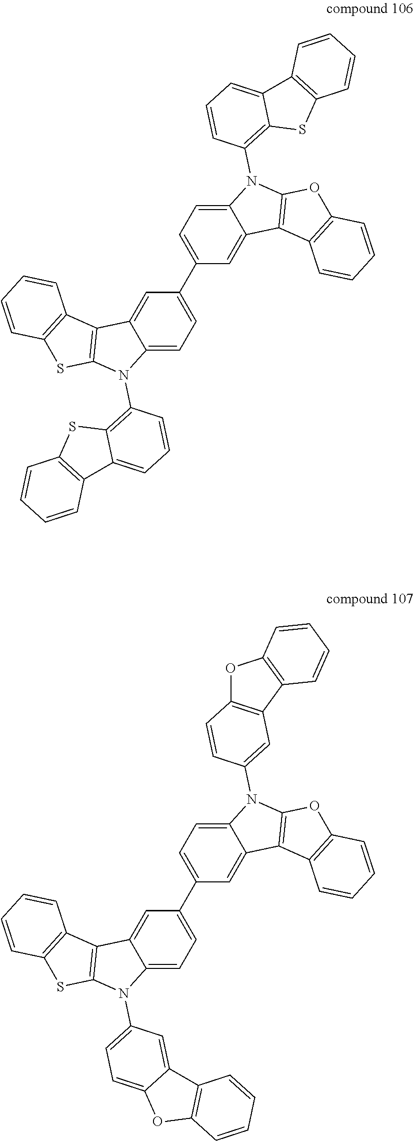 [SMILES: c1ccc2c(c1)oc1c2c2cc(-c3ccc4c(c3)c3c5ccccc5sc3n4-c3cccc4c3sc3ccccc34)ccc2n1-c1cccc2c1sc1ccccc12 ; c1ccc2c(c1)oc1ccc(-n3c4ccc(-c5ccc6c(c5)c5c7ccccc7sc5n6-c5ccc6oc7ccccc7c6c5)cc4c4c5ccccc5oc43)cc12]

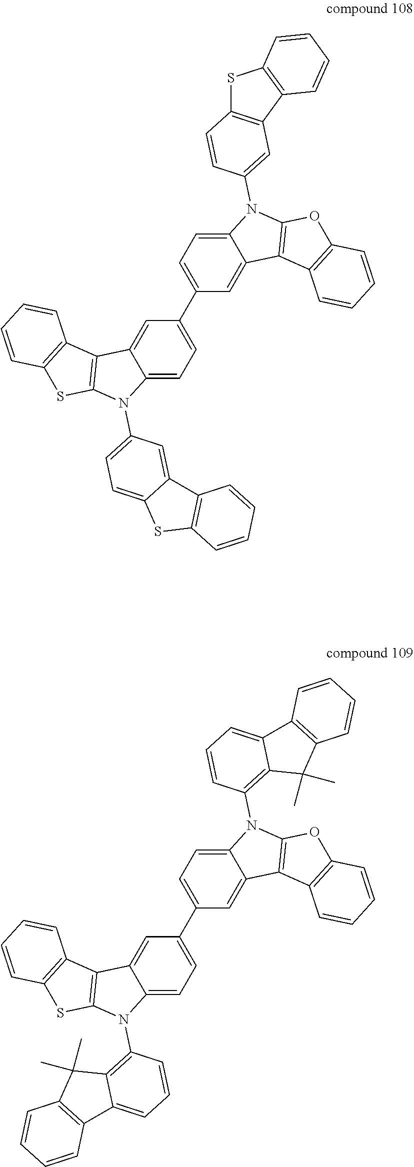 [SMILES: c1ccc2c(c1)oc1c2c2cc(-c3ccc4c(c3)c3c5ccccc5sc3n4-c3ccc4sc5ccccc5c4c3)ccc2n1-c1ccc2sc3ccccc3c2c1 ; CC1(C)c2ccccc2-c2cccc(-n3c4ccc(-c5ccc6c(c5)c5c7ccccc7sc5n6-c5cccc6c5C(C)(C)c5ccccc5-6)cc4c4c5ccccc5oc43)c21]

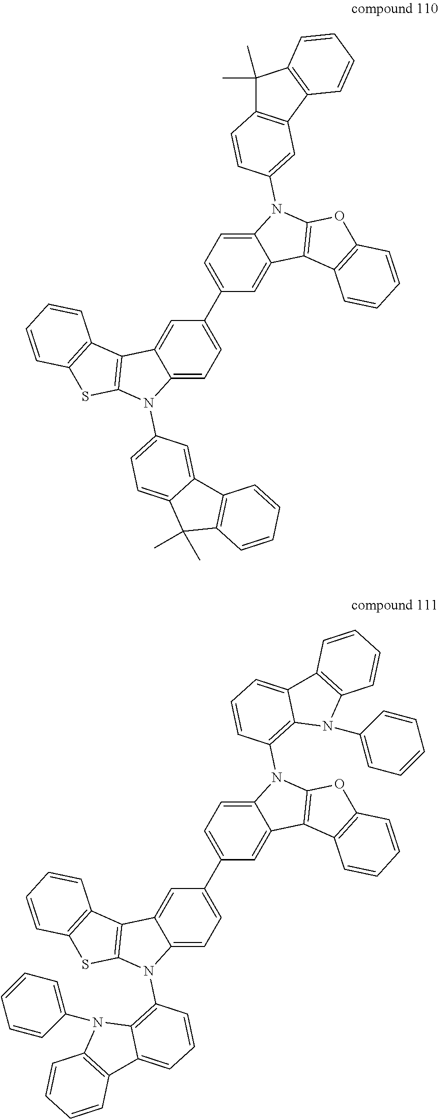 [SMILES: CC1(C)c2ccccc2-c2cc(-n3c4ccc(-c5ccc6c(c5)c5c7ccccc7sc5n6-c5ccc6c(c5)-c5ccccc5C6(C)C)cc4c4c5ccccc5oc43)ccc21 ; c1ccc(-n2c3ccccc3c3cccc(-n4c5ccc(-c6ccc7c(c6)c6c8ccccc8sc6n7-c6cccc7c8ccccc8n(-c8ccccc8)c67)cc5c5c6ccccc6oc54)c32)cc1]

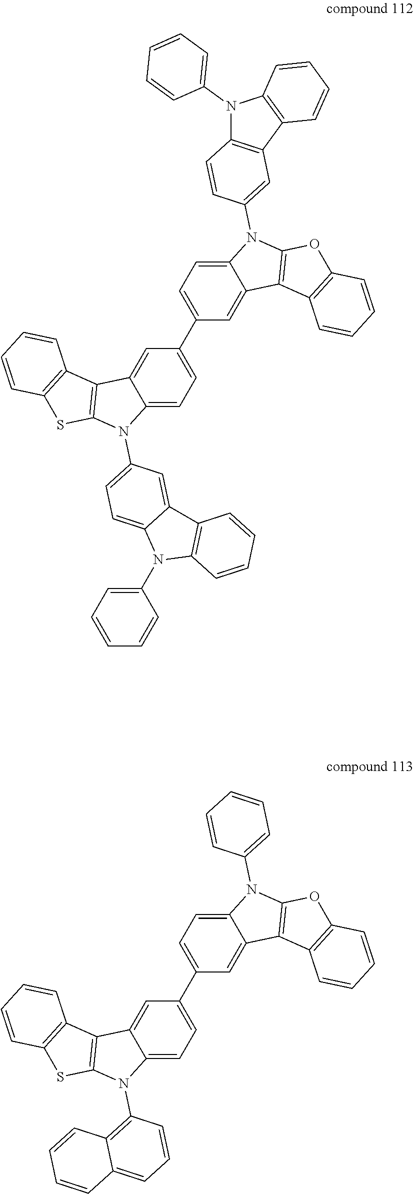 [SMILES: c1ccc(-n2c3ccccc3c3cc(-n4c5ccc(-c6ccc7c(c6)c6c8ccccc8sc6n7-c6ccc7c(c6)c6ccccc6n7-c6ccccc6)cc5c5c6ccccc6oc54)ccc32)cc1 ; c1ccc(-n2c3ccc(-c4ccc5c(c4)c4c6ccccc6sc4n5-c4cccc5ccccc45)cc3c3c4ccccc4oc32)cc1]

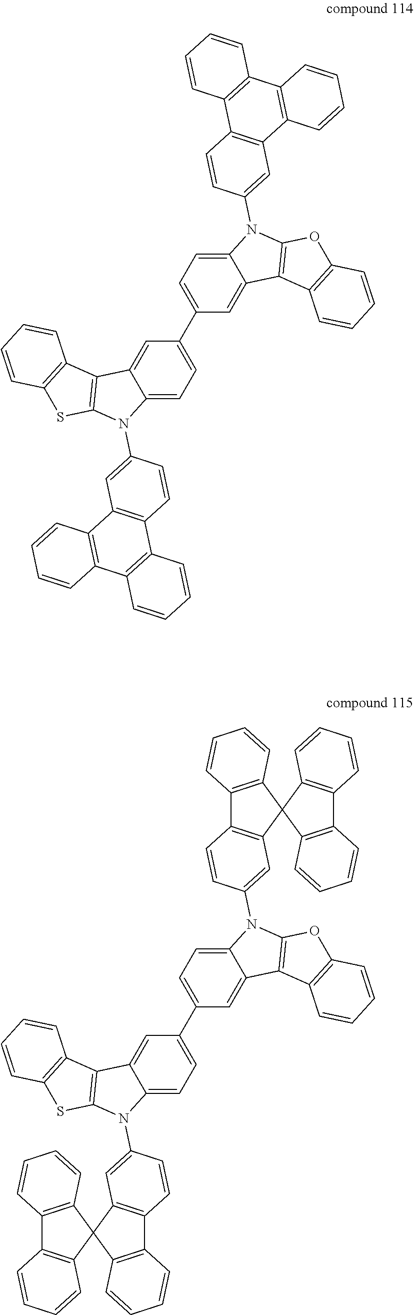 [SMILES: c1ccc2c(c1)oc1c2c2cc(-c3ccc4c(c3)c3c5ccccc5sc3n4-c3ccc4c5ccccc5c5ccccc5c4c3)ccc2n1-c1ccc2c3ccccc3c3ccccc3c2c1 ; c1ccc2c(c1)-c1ccccc1C21c2ccccc2-c2ccc(-n3c4ccc(-c5ccc6c(c5)c5c7ccccc7sc5n6-c5ccc6c(c5)C5(c7ccccc7-c7ccccc75)c5ccccc5-6)cc4c4c5ccccc5oc43)cc21]

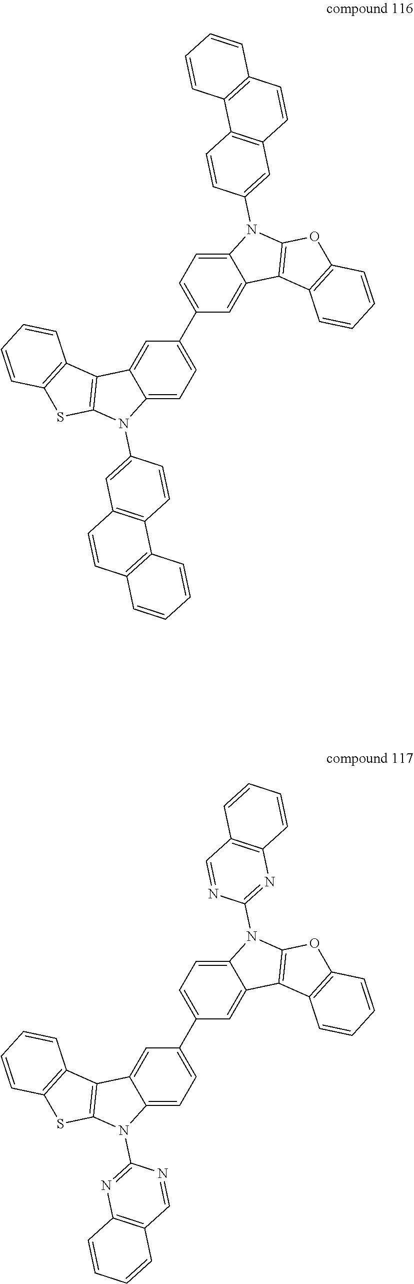 [SMILES: c1ccc2c(c1)ccc1cc(-n3c4ccc(-c5ccc6c(c5)c5c7ccccc7sc5n6-c5ccc6c(ccc7ccccc76)c5)cc4c4c5ccccc5oc43)ccc12 ; c1ccc2nc(-n3c4ccc(-c5ccc6c(c5)c5c7ccccc7sc5n6-c5ncc6ccccc6n5)cc4c4c5ccccc5oc43)ncc2c1]

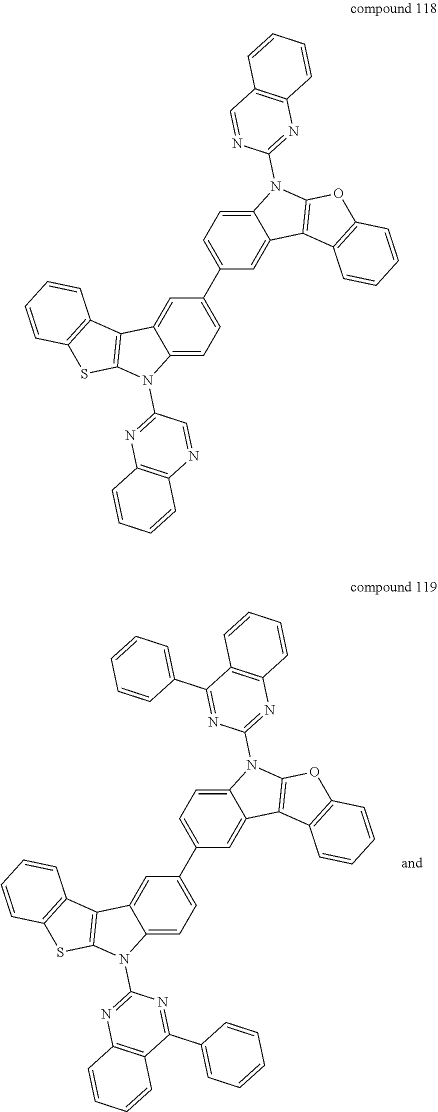 [SMILES: c1ccc2nc(-n3c4ccc(-c5ccc6c(c5)c5c7ccccc7sc5n6-c5cnc6ccccc6n5)cc4c4c5ccccc5oc43)ncc2c1 ; c1ccc(-c2nc(-n3c4ccc(-c5ccc6c(c5)c5c7ccccc7sc5n6-c5nc(-c6ccccc6)c6ccccc6n5)cc4c4c5ccccc5oc43)nc3ccccc23)cc1]

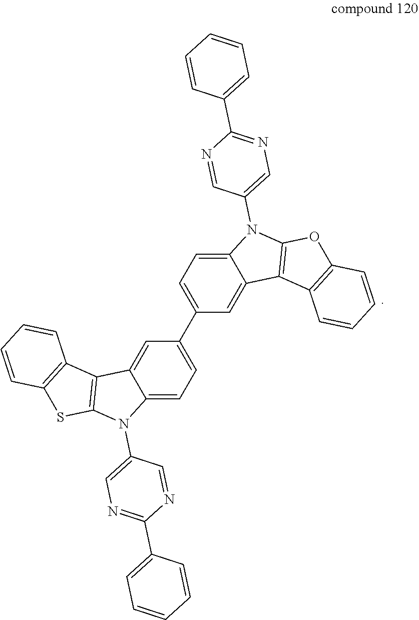 [SMILES: c1ccc(-c2ncc(-n3c4ccc(-c5ccc6c(c5)c5c7ccccc7sc5n6-c5cnc(-c6ccccc6)nc5)cc4c4c5ccccc5oc43)cn2)cc1]

8. An organic electroluminescence device comprising:
an anode:
a cathode: and
an organic layer, disposed between the anode and the cathode, comprising an organic compound of formula (1): 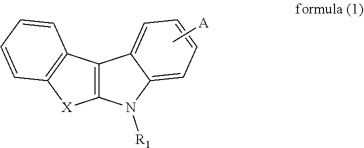
wherein A represents mono to the maximum allowable substitution;
wherein each A comprises at least one chemical group selected from the group consisting of 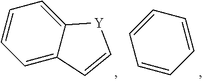
and combinations thereof;
wherein each Y is divalent bridge selected from the group consisting from O, S, CR₇R₈ and NR₉;
wherein X is a divalent bridge selected from the group consisting of O, S and NR₆; and
wherein R₁, R₆, R₇, R₈, and R₉ are independently hydrogen or a substituent selected from the group consisting of alkyl, aralkyl, aralkyl, heteroaryl, deuterium, halide, cycloalkyl, heteroalkyl, arylalkyl, alkoxy, aryloxy, amino, silyl, alkenyl, cycloalkenyl, heteroalkenyl, alkynyl, acyl, carbonyl, carboxylic acids, ester, nitrile, isonitrile, sulfanyl, sulfinyl, sulfonyl, phosphino, and combinations thereof.

9. The organic electroluminescence device of claim 9, wherein the organic layers comprise an emissive layer having a host, and wherein the organic compound is comprised as the host.

10. The organic electroluminescence device of claim 9, wherein the organic layers comprise a hole transporting layer, and wherein the organic compound of claim 1 is comprised as the hole transporting layer.

11. The organic electroluminescence device of claim 9, wherein the organic layers comprise an electron transporting layer, and wherein the organic compound of claim 1 is comprised as the electron transporting layer.

12. The organic electroluminescence device of claim 9, wherein the organic layers comprise an electron blocking layer, and wherein the organic compound of claim 1 is comprised as the electron blocking layer.

13. The organic electroluminescence device of claim 9, wherein the organic layers comprise a hole blocking layer, and wherein the organic compound of claim 1 is comprised as the hole blocking layer.

14. The organic electroluminescence device of claim 9, wherein the organic electroluminescence device is a lighting panel.

15. The organic electroluminescence device of claim 9, wherein the organic electroluminescence device is a backlight panel.
